(19) United States
(12) Patent Application Publication
(10) Pub. No.: US 2025/0376691 A1
(43) Pub. Date: Dec. 11, 2025

(54) FLOTATION OF CULTURED EMBRYO EXPLANTS FOR IMPROVED PLANT REGENERATION EFFICIENCY

(71) Applicant: Monsanto Technology LLC, St. Louis, MO (US)

(72) Inventors: Yurong Chen, Chesterfield, MO (US); Christopher Daniel, Wentzville, MO (US); Erica Renfro, Imperial, MO (US)

(21) Appl. No.: 18/877,049

(22) PCT Filed: Jun. 21, 2023
(86) PCT No.: PCT/US2023/068779

§ 371 Date: Dec. 19, 2024

Related U.S. Application Data
(60) Provisional application No. 63/355,045, filed on Jun. 23, 2022.

(51) Int. Cl. C12N 15/82; A01H 4/00

(52) U.S. Cl. CPC C12N 15/8205; A01H 4/008; C12N 15/8295

(57) ABSTRACT

The disclosure provides novel methods for improving the regeneration throughput and/or plugging frequency of monocot embryo explants by flotation and selective collection of viable and regenerable explants. Genetic modification or transformation of monocot plants may include preparation of seed excised embryo explants, rehydration, inoculation and co-culture, bud induction, extended bud induction, and regeneration of genetically modified plants or plant parts. To improve regeneration and plugging frequency, cultured monocot seed excised embryo explants following the bud induction or extended bud induction step are placed in a flotation medium to separate the explants into top and bottom layers or fractions, such that the top fraction can be selectively collected and advanced to regeneration media. The top fraction is shown to have a higher or similar plugging frequency than the bottom fraction and thus the flotation step reduces costs and improves transformation throughput by removing unproductive explants in the bottom fraction having a much lower regeneration and plugging frequency.

### CROSS-REFERENCE TO RELATED APPLICATIONS

This application claims the priority of U.S. Provisional Appl. Ser. No. 63/355,045, filed Jun. 23, 2022, the entire disclosure of which is incorporated herein by reference.

### FIELD OF THE INVENTION

The present disclosure relates to compositions and methods for improving the efficiency of genetically modifying monocot seed excised embryo explants.

### BACKGROUND OF THE INVENTION

Monocot plants, such as corn, wheat, rice, barley, and sorghum, are important crops and are primary food sources in many areas of the world. Biotechnology methods have been used to improve these crops by the creation of novel traits through genetic modifications of plants, which often rely on the delivery of polynucleotide molecules to plant cells to produce genetically modified plants or plant parts having the improved traits or characteristics. However, there is a continuing need in the art for improved methods of genetically modifying a plant, particularly a monocot plant, that do not rely on the use of callus culture, that can be performed more efficiently, and that are less plant germplasm dependent.

The inventions described herein provide novel systems and methods for the improved transformation or genetic modification of monocot seed excised embryo explants and the regeneration of genetically modified plants or plant parts therefrom with increased efficiency and a reduction in time and/or space requirements in the laboratory and controlled environment.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 demonstrates transient gene expression of transformed corn explants exposed to a gravitational force either prior to or during Agrobacterium inoculation.

FIG. 2 demonstrates transient gene expression of transformed corn explants exposed to a gravitational force during Agrobacterium inoculation at various temperatures.

FIG. 3 demonstrates transient gene expression of transformed wheat explants exposed to various gravitational forces during Agrobacterium inoculation.

FIG. 4 shows images of the effect of different gravitational forces (291 g, 1164 g, 2619 g and 4657 g) during inoculation on transient GUS expression in two experiments.

FIG. 5 shows Agrobacterium transformation of crushed Setaria seeds. FIG. 5A shows regenerated shoots 8 weeks after inoculation; FIG. 5B shows leaves from the regenerated shoots with GUS expression after 1 hour in X-gluc solution, confirming transformation; FIG. 5C shows a Southern hybridization of DNA isolated from the regenerated plants grown in the greenhouse (see example of plant in FIG. 5D) using a probe specific to the CP4 gene confirming that transgenic plants contained a single copy of the CP4 transgene.

FIG. 6 shows a seed rolling assembly (FIG. 6A) for removal of seed coat from Setaria seeds; FIG. 6B shows Setaria seeds before removal of seed coat; and FIG. 6C shows Setaria seeds after removal of seed coat.

FIG. 7 shows the progression of Agrobacterium-mediated transformation, culturing and regeneration of plants from intact Setaria seeds. FIG. 7A shows Setaria seeds after Agrobacterium infection; FIG. 7B shows GUS expression around the meristem region after X-gluc staining; FIG. 7C shows newly formed buds around the meristem region near leaf base where GUS staining was observed; and FIG. 7D shows regenerated plants following selection and rooting.

FIG. 8 shows GUS expression in reproductive tissues and in R1 seedlings of transgenic Setaria viridis plants. The top panels in FIG. 8 show GUS expression in anthers and pollen stigma (top left); spikelet (top middle); and immature seed (top right). The bottom panels in FIG. 8 show GUS expression in seedlings for three different transgenic events (Events 1, 4, and 3).

FIG. 9 shows the separation of explants into two layers in (A) a 600 mL beaker and (B) a Vivi® tissue culture vessel.

FIG. 10 shows images of explants cultured in regeneration media from the top floating layer (A) and the bottom non-floating layer (B) of the flotation step.

### DETAILED DESCRIPTION OF THE INVENTION

The following is a detailed description provided to aid those skilled in the art in practicing the present inventions. Modifications and variations to the embodiments described herein can be made without departing from the spirit or scope of the present inventions. Compositions and methods are provided for improving the transformation of monocot seed excised embryo explants, which may include one or more steps of explant preparation, explant rehydration, Agrobacterium inoculation and co-culture, bud induction, extended bud induction, and/or regeneration of genetically modified plants or plant parts as described herein.

### A. Explant Preparation

In accordance with embodiments of the present invention, one or more monocot seed embryo explants can be produced from monocot plant seeds for production of genetically modified monocot plants or plant parts. Such plant seeds may be taken or harvested from plants grown in a field or greenhouse. Such monocot plant seeds can be a mature or immature monocot plant seeds but may preferably be mature monocot plant seeds. Examples of monocot plants include, but are not limited to, corn plants, wheat plants, rice plants, barley plants, rye plants, millet plants, oat plants, turfgrass plants, and sorghum plants. Examples of monocot plant seeds include, but are not limited to, corn or maize seeds, wheat seeds, rice seeds, barley seeds, rye seeds, millet seeds, oat seeds, turfgrass seeds, and sorghum seeds. Use of mature monocot plant seeds may provide the benefits or advantages of improved seed storage, explant preparation, and/or culturing. Examples of monocot plants or plant seeds that may be genetically modified or transformed according to present embodiments include any plant species within the Poaceae or Gramineae family of monocot or cereal plants and grasses, which may include any Zea genus corn or maize species, such as Zea mays, any Oryza genus rice species, such as Oryza sativa, any Triticum genus wheat species, such as Triticum aestivum or Triticum turgidum L. var. durum, any Hordeum genus barley species, such as Hordeum vulgare, any Avena genus oat species, such as Avena sativa, any Sorghum genus sorghum species, such as Sorghum bicolor or Sorghum vulgare, any Secale genus rye species, such as Secale cereale, any Saccharum sugarcane species, or any Setaria, Pennisetum, Eleusine, Echinochloa, or Panicum genus millet species, such as Setaria viridis, Setaria italica, Pennisetum glaucum, Eleusine coracana, Echinochloa frumentacea, Panicum sumatrense, or Panicum miliaceum.

According to some embodiments, methods and compositions are provided for preparing, culturing, selecting and using explants, as well as the explants or cultured explants produced thereby. As used herein, the term “explant” or “seed embryo explant” refers to a plant part or plant tissue that is capable of being genetically modified and subsequently regenerated into a genetically modified plant or plant part. An “explant” or “seed embryo explant” may refer to a plant seed or any part of a plant seed, which may comprise at least a portion of a plant seed embryo. An “explant” or “seed embryo explant” may comprise an embryo explant excised from a plant seed that may comprise at least a part of an embryo meristem tissue. Alternatively, an “explant” or “seed embryo explant” may refer to a whole or intact plant seed, or a crushed, deformed or partially opened plant seed that may be produced by any suitable mechanical process. As used in reference to an explant or seed embryo explant, “partially opened” refers to an altered state of a plant seed that has one or more openings or fissures in the plant seed introduced by a mechanical force, such as squeezing, crushing, rolling, pressing, extruding, etc. An explant or seed embryo explant that is a whole or intact plant seed or a crushed, deformed or partially opened plant seed may in many cases have its seed coat removed. As used herein, a “genetically modified” plant, plant part, plant tissue, explant, or plant cell comprises a genetic modification or transgene introduced into the genome of the plant, plant part, plant tissue, explant, or plant cell through genetic engineering, which may be via a genetic transformation or a genome editing technique. As used herein, a “transgenic” plant, plant part, plant tissue, explant or plant cell has an exogenous nucleic acid sequence, polynucleotide, expression cassette, and/or transgene integrated into the genome of the plant, plant part, plant tissue, explant, or plant cell. In certain embodiments, explants according to the invention may be produced manually or using an automated process. For example, seed tissues may be removed from a seed by cutting, grinding, abrasion, or any other similar process. Manual or automated methods for removal of unnecessary seed parts may also be carried out. Fluid, for example, can be used to move explants and separate desirable explants from debris during mechanized handling of seeds, including compressed air, other gases, and liquids. Embryo explants may be excised from dry, dried, or wet seeds. Mature pant seeds may become drier as part of their normal maturation process, although seeds may be further dried prior to excision and/or explants may be dried after excision from seeds. Dry or dried excision of plant embryo explants may be performed for their immediate use or for later use after storage for a period of time. Explant preparation may further comprise drying the seed and/or explant to obtain a desired moisture content of the seed and/or explant for improved storage preparation (e.g., excision) or use, depending upon the initial moisture content of the seed or explant without drying. Following excision, the explant may be purified or isolated from other seed material and debris by rinsing, flotation, or other methods known in the art.

In other embodiments, a seed or explant prepared or used in accordance with the embodiments of the present inventions may be defined as having an internal moisture of about 3% to about 25%, about 3% to about 20%, about 3% to about 15%, about 3% to about 10%, about 5% to about 10%, including about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or about 25% internal moisture, including all ranges derivable therebetween. An explant may be produced from a mature seed having a moisture content as described herein. In specific aspects, the moisture content of the seed or explant may be measured prior to or after explant excision, explant storage, while an explant is in storage, prior to explant rehydration, and/or prior to genetic modification or transformation.

In one aspect, any monocot embryo explant may be prepared or used according to the embodiments of the present inventions. In particular embodiments, the monocot embryo explant may be a mature embryo, an immature embryo, meristematic tissue, callus tissue, or any other tissue that is transformable and regenerable. In some embodiments the mature embryo explant is a dry excised explant. Dry excised explants may be taken from seeds and used almost directly as targets for transformation or genetic modification. In one embodiment, dry excised explants may be taken from mature dry seeds and used as targets for transformation or genetic modification with perhaps only minimal wetting, hydration, or pre-culturing steps. In other embodiments wet, dried wet, or wet excised embryo explants may be used as a target for transformation or genetic modification. As used herein “wet” embryo explants refer to dry excised explants subjected to wetting, hydration, imbibition, or other minimal culturing steps prior to transformation or genetic modification. As used herein “dried wet” embryo explants refer to embryo explants which are primed for germination by wetting and then dried to arrest germination. As used herein “wet excised” explants refer to explants excised from imbibed or hydrated seeds. A wet embryo explant is hydrated or imbibed after excision from a seed, whereas a wet excised embryo explant is excised from an already hydrated or imbibed seed. As used herein a “callus” refers to a proliferating mass of unorganized, undifferentiated and/or dedifferentiated plant cells or tissue.

Explants for use according to the present invention may be genetically modified at various times after isolation, excision or removal from the mature monocot seed. In one embodiment, explants may have been removed from seeds for less than a day, for example, from about 1 to about 24 hours, such as about 1, 2, 3, 5, 7, 8, 9, 10, 11, 12, 13, 14, 15, 16, 17, 18, 19, 20, 21, 22, 23 or 24 hours prior to use. In other embodiments, explants may be stored for longer periods, including days, weeks, months, or years prior to use. Methods and parameters for drying, storing, and germinating seed are known in the art (e.g., U.S. Pat. No. 8,362,317, specifically incorporated herein by reference in its entirety, Senaratna et al., 1983, Pl. Physiol. 72:620-624, 1983; Vertucci and Roos, 1990, Pl. Physiol. 90:1019-1023, 1990; Chai et al., 1998, Seed Science Research 8 (Supplement 1): 23-28, 1998). Any conditions may be used as desired, including incubation or storage at temperatures, for example, of about −80° C. to about 60° C.

The invention may in certain aspects involve sterilization of seeds or explants. Sterilization can include contacting seed or explant material with various liquid or gases that serve to reduce or eliminate the presence of viable bacterial or fungal contaminants that could otherwise interfere with seed or embryo viability. Sterilization by application of liquid may also hydrate or partially hydrate the plant seeds, explants, embryos or tissues and serve the purpose of priming the seeds, explants, embryos or tissues. Methods for sterilization include, but are not limited to, the use of chlorine gas, ozone, solutions of bleach or alcohol, ultraviolet light, temperatures of −20° C. or lower, and exposure to a temperature higher than 40° C.

In one aspect of the present invention, explants may be rehydrated prior to transformation or genetic modification. Rehydration media or solutions are known in the art and may comprise, for example, water, basal salts, macronutrients, micronutrients, and/or vitamins. The rehydration medium will typically not contain any plant hormones, such as an auxin or cytokinin. In one embodiment, rehydrating monocot seed embryo explants may be carried out for a period of time in a range from about 30 minutes to about 24 hours prior to transformation or genetic modification or any length of time within such range, such as for about 1 hour, about 1.5 hours, about 2 hours, or about 2.5 hours, or less than or equal to about 3 hours or less than or equal to about 2.5 hours, or less than or equal to about 2 hours, or in a range of about 1 hour to about 3 hours, or in a range of about 1 hour to about 2.5 hours, or in a range of about 1.5 hours to about 2.5 hours. Rehydration of embryo explants prior to transformation or genetic modification may improve transformation or editing frequency or the recovery of transformed or edited plants, as compared to explants that were not rehydrated, by at least about 0.2-fold, 0.4-fold, 0.6-fold, 0.8 fold, 1-fold, 2-fold, 3-fold, 4-fold, 5-fold, 10-fold, 15-fold, 20-fold, 25-fold, 30-fold, 35-fold, or 40-fold, including all ranges derivable therebetween. In a particular embodiment, rehydrating monocot seed embryo explants for at least about 2 hours prior to transformation or genetic modification may improve transformation or editing frequency or the recovery of transformed or edited plants, as compared to explants rehydrated for about 1 hour, by at least about 0.2-fold, 0.4-fold, 0.6-fold, 0.8-fold, 1-fold, 2-fold, 3-fold, 4-fold, 5-fold, or 10-fold, including all ranges derivable therebetween.

Improved transformation or editing of plants may be measured, in some embodiments of the present disclosure, by genotyping, transient expression, shoot frequency, percentage of normal shoots, normal shoot frequency, normal plant frequency, percentage of rooted shoots at one or more different steps or plant pulls, and/or overall, transformation frequency, plugging frequency, frequency of low copy number plants, and/or low copy number frequency. The methods described herein may, for example, improve transformation or editing of plants by at least about 0.1-fold, 0.2-fold, 0.4-fold, 0.6-fold, 0.8 fold, 1-fold, 1.5-fold, 2-fold, 2.5-fold, 3-fold, 3.5-fold, 4-fold, 4.5-fold, 5-fold, 7.5-fold, 10-fold, 15-fold, 20-fold, 30-fold, or 40-fold, including all ranges derivable therebetween.

### B. Inoculation and Force-Assisted Transformation

Methods and compositions are provided herein for genetic transformation or modification of monocot seed derived explants or monocot embryo explants. These explants may be defined, in one aspect or embodiment, as comprising meristematic tissue or embryonic meristem tissue, which contains plant cells that can differentiate or develop to produce multiple plant structures including, but not limited to, stem, roots, leaves, germ line tissue, and seeds. Indeed, an embryo explant may be defined as comprising all or part of a seed embryo removed from other non-embryonic seed tissues and further comprising all or part of a meristematic tissue or embryonic meristem tissue. Embodiments of the present inventions may include genetically transforming or modifying at least one cell of the explant by introducing a heterologous polynucleotide molecule into the at least one cell by any suitable method or technique known in the art, such as electroporation, microprojectile or particle bombardment, microinjection, PEG-mediated transformation, Rhizobiales- or Agrobacterium-mediated transformation, and other modes of direct DNA uptake. All or part of the heterologous polynucleotide may then be transformed or incorporated into the genome of the plant cell, expressed into one or more editing molecules or tools (such as a guide RNA and/or site-specific nuclease), and/or provide a template for editing or site-directed integration. According to many embodiments, the heterologous polynucleotide is introduced into the at least one explant cell via Rhizobiales- or Agrobacterium-mediated transformation. The introducing or inoculation step may be carried out under ambient lighting conditions and may include subjecting the embryo explant to any force treatment.

According to embodiments of the present inventions, a force treatment is applied to the monocot seed embryo explant either prior to or during inoculation, or prior to and during inoculation, of the monocot seed embryo explant with a Rhizobiales or Agrobacterium bacterium comprising the heterologous polynucleotide molecule. According to some embodiments, the force treatment is applied during and/or after rehydration of the monocot seed embryo explant. According to many embodiments, the force treatment can be applied during the inoculation step while the monocot seed embryo explant is in contact with the inoculation medium. In one embodiment, explants “in contact with” a medium may be positioned completely or partially in or on a medium. Non-limiting examples of medium in which an explant may be in contact with include a liquid medium, a solid medium, and a substrate comprising a medium. The monocot seed embryo explant may be submerged in a volume of the inoculation medium when the force treatment is applied. Alternatively, the force treatment may be applied to the monocot seed embryo explant after an excess amount of the inoculation medium has been removed. The inoculation medium, for example, may be decanted, poured, or blotted from the explant prior to application of the force treatment. If the force treatment is applied during the inoculation step, then the inoculation medium may not be entirely absent from contacting the monocot seed embryo explant, even if an amount or volume of the inoculation medium is removed from the explant before the force treatment.

As used herein, the term “heterologous polynucleotide molecule” refers to a polynucleotide molecule that is not naturally present, or is not naturally present in the same form, structure, etc., in the cell being transformed, without human intervention. For example, a heterologous polynucleotide molecule may not naturally occur in the plant species being transformed, or may be expressed in a manner or genomic context that differs from the natural expression pattern or genomic context found in the species being transformed, (e.g., overexpressed). In one embodiment, the heterologous polynucleotide molecule may be the combination of two or more polynucleotide molecules, wherein such a combination is not normally found in nature. The two polynucleotide molecules may, in certain embodiments, be derived from different species or may be derived from different genes, such as, different genes from the same species or the same genes from different species. In particular embodiments, a heterologous polynucleotide molecule may comprise two polynucleotide sequences that are not found juxtaposed or operably linked in any naturally occurring polynucleotide molecule. In one embodiment, the heterologous polynucleotide molecule may comprise a promoter or other regulatory sequence operably linked to a transcribable polynucleotide sequence, wherein the promoter and the transcribable polynucleotide sequence are not operably linked in any naturally occurring polynucleotide molecule. As used herein, the term “polynucleotide molecule” refers to a linear or circular single-stranded or double-stranded DNA or RNA polynucleotide molecule or sequence, which may be derived from any source. For example, a polynucleotide molecule may comprise a polynucleotide sequence in which one or more nucleic acid sequences have been linked together in a functionally operative manner. As used herein the term “nucleic acid sequence” refers to deoxyribonucleic acid (DNA) or ribonucleic acid (RNA) sequence.

According to some embodiments, the polynucleotide molecule or heterologous polynucleotide molecule may be a recombinant polynucleotide molecule. As used herein, the term “recombinant” when used in reference to a polynucleotide (DNA or RNA) molecule, protein, construct, vector, etc., refers to a polynucleotide or protein molecule or sequence that is not naturally present, or is not naturally present in the same form, structure, etc., without human intervention. In one embodiment a recombinant polynucleotide (DNA or RNA) molecule, protein, construct, vector, etc. may comprise, for example, a combination of two or more polynucleotide or protein sequences that do not naturally occur together in the same manner, such as a polynucleotide molecule, protein, construct, etc., comprising at least two polynucleotide or protein sequences that are operably linked but heterologous with respect to each other. In another embodiment, a recombinant polynucleotide (DNA or RNA) molecule, protein, construct, vector, etc. may comprise, for example, any combination of two or more polynucleotide or protein sequences in the same molecule (e.g., a plasmid, construct, vector, chromosome, protein, etc.) where such a combination is man-made and not normally found in nature. As used in this definition, the phrase “not normally found in nature” means not found in nature without human intervention. A recombinant polynucleotide or protein molecule, construct, etc., may comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., may also refer to a polynucleotide or protein molecule or sequence that has been genetically engineered and/or constructed outside of a cell. For example, a recombinant DNA molecule may comprise any engineered or man-made plasmid, vector, etc., and may include a linear or circular DNA molecule. Such plasmids, vectors, etc., may contain various maintenance elements including, for example, a prokaryotic origin of replication and selectable marker, as well as one or more transgenes or expression cassettes perhaps in addition to a plant selectable marker gene, etc.

To improve transformation or editing of plants, a variety of different force treatments may be used or applied to the monocot seed embryo explant before and/or during the inoculation step, such as a centrifugal force treatment, a gravitational force treatment, a vacuum treatment, a sonication treatment, a vortexing treatment, a shearing treatment, a mechanical force treatment, and/or a pressure treatment, or any combination thereof. According to some embodiments, a force treatment may comprise a pressure treatment and/or a gravitational (or centrifugal) force treatment. According to some embodiments, a force treatment may comprise a pressure treatment. According to some embodiments, a force treatment may comprise a gravitational (or centrifugal) force treatment. In specific embodiments, the methods described herein may further comprise applying a mechanical force treatment, a vortexing treatment, a shaking or shearing treatment, a sonication treatment, and/or a vacuum treatment, in addition to a pressure treatment and/or a gravitational (or centrifugal) force treatment. Without being bound by theory, application of a force treatment prior to or during inoculation may improve transformation by increasing the contact and attachment of the Rhizobiales bacterium to the monocot seed embryo explant, by wounding the monocot seed embryo explant and/or by increasing the permeation of the Rhizobiales bacterium into meristematic or other explant tissues.

In some embodiments, the force treatment may comprise applying a pressure force or treatment in a range from about 100 pounds per square inch (psi) to about 20,000 psi, about 100 psi to about 18,000 psi, about 100 psi to about 16,000 psi, about 100 to about 14,000 psi, about 100 to about 12,000 psi, about 100 to about 10,000 psi, about 100 to about 8,000 psi, about 100 to about 6,000 psi, about 100 to about 4,000 psi, about 100 to about 2,000 psi, about 100 to about 1,000 psi, or about 100 psi to about 500 psi, such as about 100 psi, about 150 psi, about 200 psi, about 250 psi, about 300 psi, about 350 psi, about 400 psi, or about 500 psi, of pressure to the monocot seed embryo explant, including all ranges derivable therebetween. Other units for pressure are also known in the art. Methods for converting pressure in psi to other units, for example, standard atmospheres (atm) and Newtons (N) per square meter (N/m²) are known in the art. Pressure in atm can be accurately calculated using the following formula: atm=pressure (psi)/14.6959488, and 1 psi equals about 6894.76 N/m². Therefore, 100 psi is equal to about 6.80 atm, and 20,000 psi is equal to about 1360.9 atm. The pressure treatment can also be converted to an amount of force when the surface area is known or fixed. For example, the surface area of piston/cell cavity of the French Press 40K pressure cell (Thermo® IEC. FA-032) used in the Examples herein is about 0.88 in². Therefore, 3,334 psi applied using the French Press 40K pressure cell is equal to about 13,000 N [(3,334 psi×0.88 in²)]/[0.225 pounds/N] The pressure treatment, in some embodiments, may be applied from about 10 seconds to about 10 minutes, from about 15 seconds to about 8 minutes, from about 30 seconds to about 6 minutes, from about 2 minutes to about 4 minutes, or for about 3 minutes, including all ranges derivable therebetween.

The methods described herein comprise applying a gravitational or centrifugal force in a range from about 100×g to about 10,000×g, about 100×g to about 5,000×g, about 250×g to about 5,000×g, about 500×g to about 5,000×g, about 500×g to about 3,000×g, about 600×g to about 2,700×g, such as about 500×g, about 550×g, about 600×g, about 650×g, about 700×g, about 750×g, about 800×g, about 850×g, about 900×g, about 950×g, about 1000×g, about 1500×g, about 2000×g, about 2500×g, about 3000×g, about 3500×g, or about 4000×g, may be applied to the monocot seed embryo explant, including all ranges derivable therebetween. A non-limiting example of a gravitation force treatment which may be applied to the monocot seed embryo explant includes a centrifugal force or relative centrifugal force, which may be applied using an appropriate centrifuge. Methods for converting gravitational or centrifugal force, such as the relative centrifugal force (RCF) created by a centrifuge, to other units, such as revolutions per minute (rpm) and newton (N), are known in the art. Relative centrifugal force can be calculated based on the rpm and known dimensions of the device using the following formula: rpm=√[RCF/(r×1.118)×1×10⁵], wherein r=the rotational radius in centimeters. For the Sorvall™ RC3BP centrifuge (Thermo Fisher Scientific, Waltham, MA, USA) used in the Examples described herein, the rotational radius is about 24.67 cm. Therefore, 2620×g is equal to about 3,082 rpm [√[2620/(24.67×1.118)]×1×10⁵]. Similarly, centrifugal force in Newtons can be accurately estimated using the following formula: Force (N)=RCF×mass of the contents of the centrifugation tube (kg)×9.82 m/s². In one embodiment, if the mass of contents of the centrifugation tube may be about 0.05 kg, then 2620×g would be equal to about 1286 N [(2620×g)×0.05 kg×9.82 m/s²]. The gravitational or centrifugal force treatment may be applied, in some embodiments, in a range from about 1 minute to about 2 hours, about 2 minutes to about 110 minutes, about 5 minutes to about 90 minutes, about 10 minutes to about 90 minutes, about 10 minutes to about 80 minutes, about 10 minutes to about 70 minutes, about 10 minutes to about 60 minutes, about 10 minutes to about 50 minutes, about 15 minutes to about 45 minutes, or about 20 minutes to about 40 minutes, such as about 10 minutes, about 15 minutes, about 20 minutes, about 25 minutes, about 30 minutes about 35 minutes, about 40 minutes, about 45 minutes, about 50 minutes, about 55 minutes, or about 60 minutes (1 hour), including all ranges derivable therebetween.

The force treatment, such as the gravitational (or centrifugal) and/or pressure treatment(s), may be applied at a temperature of about 0.5° C. to about 28° C., about 2° C. to about 28° C., about 4° C. to about 28° C., about 10° C. to about 28° C., about 10° C. to about 25° C., or about 15° C. to about 23° C., including all ranges derivable therebetween.

The application of a force treatment as described herein may, in some embodiments, improve transformation or editing of plants by at least about 0.2-fold, 0.4-fold, 0.6-fold, 0.8 fold, 1-fold, 1.5-fold, 2-fold, 2.5-fold, 3-fold, 3.5-fold, 4-fold, 4.5-fold, or 5-fold, including all ranges derivable therebetween.

In one aspect of the methods provided herein, the force treatment may comprise applying both a pressure treatment and a gravitational force treatment to the monocot seed embryo explant. The pressure treatment and/or the gravitational force treatment may be applied prior to, during, or prior to and during inoculation of a monocot seed embryo explant with a bacterium from the order Rhizobiales, wherein the Rhizobiales bacterium comprises a heterologous polynucleotide for transforming, editing or genetically modifying at least one plant cell of the monocot seed embryo explant. In some embodiments, the pressure treatment is applied prior to applying the gravitational force treatment. In other embodiments, the gravitational force treatment is applied prior to the pressure treatment. The order of application of a pressure treatment and a gravitational force treatment may be preferred based on improved transformation or editing efficiency or frequency or based on case of handling. In some embodiments, when a combination of pressure and gravitational force treatments are applied to monocot seed embryo explants, the pressure treatment may be applied before the gravitational force treatment, which may be due at least in part to the ability to apply the force treatment more evenly prior to pelleting the explants with the gravitational or centrifugal treatment. However, the centrifuged or pelleted explants could potentially be resuspended prior to a subsequent pressure treatment, or the pressure treatment could be applied to the centrifuged or pelleted explants without resuspension. In an aspect of the present inventions, applying a pressure treatment and a gravitational force treatment either prior to, during, or prior to and during inoculation may improve transformation or editing of plants by at least about 0.2-fold, 0.4-fold, 0.6-fold, 0.8 fold, 1-fold, 1.5-fold, 2-fold, 2.5-fold, 3-fold, 3.5-fold, 4-fold, 4.5-fold, or 5-fold, including all ranges derivable therebetween, as compared to applying only the pressure treatment or only the gravitational force treatment.

In another aspect, the methods described herein may further comprise applying a vacuum treatment to the monocot seed embryo explant. The vacuum treatment may comprise, for example, submerging the monocot seed embryo explant in a liquid inoculation medium comprising a Rhizobiales bacterium and subjecting the monocot seed embryo explant to decreased pressure followed by rapid or gradual repressurization. Alternatively, a vacuum treatment may be applied to a monocot seed embryo explant that is not submerged in a liquid inoculation medium. The vacuum treatment, in some embodiments, may be applied before the force treatment is applied, after the force treatment is applied, before the gravitational force treatment is applied, after the gravitational force treatment is applied, before the pressure treatment is applied, and/or after the pressure treatment is applied. In particular embodiments, where the force treatment comprises applying a pressure treatment and a gravitational force treatment, a vacuum treatment may be applied between applying the pressure treatment and applying the gravitational force treatment, regardless as to whether the gravitational force treatment or the pressure treatment is applied first. In one embodiment, the monocot seed embryo explant may be subjected to a vacuum treatment of about 0.05 atm to about 0.50 atm, about 0.05 atm to about 0.40 atm, about 0.05 atm to about 0.30 atm, about 0.05 atm to about 0.20 atm, about 0.05 atm to about 0.10 atm, about 0.10 atm to about 0.50 atm, about 0.10 atm to about 0.40 atm, about 0.10 to about 0.30 atm of pressure, or about 0.10 atm to about 0.20 atm of pressure, including all ranges derivable therebetween.

### C. Co-Culture of Embryo Explants

Following inoculation of a monocot seed embryo explant(s) with a Rhizobiales or Agrobacterium comprising a heterologous polynucleotide to introduce the heterologous polynucleotide into at least one cell of the monocot seed embryo explant(s), the monocot seed embryo explant(s) may be co-cultured in contact with a co-culture medium. The co-culture medium may comprise, for example, water, basal salts, macronutrients, micronutrients, and/or vitamins. However, according to present embodiments, the co-culture medium may not contain any plant hormones, such as an auxin or cytokinin and/or any surfactant or wetting agent, although a plant hormone or auxin and/or a surfactant or wetting agent may alternatively be present in the co-culture medium. In certain embodiments, a surfactant may include any surfactant or combination of surfactants known in the art, for example a detergent, a wetting agent, an emulsifier, a foaming agent, or a dispersant. In one embodiment, the surfactant maybe be Silwet® or a similar surfactant. According to some embodiments, the monocot seed embryo explant(s) may be in contact with the co-culture medium at a temperature in a range from about 15° C. to about 25° C., or from about 17° C. to about 23° C., or from about 18° C. to about 20° C., or at a temperature of about 15° C., about 16° C., about 17° C., about 18° C., about 19° C., about 20° C., about 21° C., about 22° C., about 23° C., about 24° C., or about 25° C. According to some embodiments, the monocot seed embryo explant(s) may be in contact with the co-culture medium for a time period ranging from about 1 day to about 10 days, or from about 2 days to about 10 days, or from about 2 days to about 8 days, or from about 3 days to about 8 days, or from about 4 days to about 8 days, or from about 5 days to about 7 days, such as for about 2 days, about 3 days, about 4 days, about 5 days, about 6 days, about 7 days, or about 8 days. According to many embodiments, the monocot seed embryo explant(s) may be in contact with the co-culture medium for at least 5 days or at least 6 days. According to present embodiments, the co-culture medium in contact with the monocot seed embryo explant(s) may be a solid, liquid or semi-solid medium. According to present embodiments, the monocot seed embryo explant(s) may be in contact with a matrix, paper or mesh material or substrate, such as a Whatman or other filter paper, that is wetted, filled or soaked with a liquid co-culture medium. According to present embodiments, the monocot seed embryo explant(s) may be in contact with, but not submerged in, the co-culture medium.

The co-culturing step may also be carried out under a variety of lighting conditions. While some degree of lighting may generally be used, all or part of the co-culture step may alternatively be performed in the dark. The lighting treatments may be quantified in terms of the light/dark cycle and intensity of light, which may be expressed as the Photosynthetic Photon Flux Density (PPFD) in units of μ/m²·s. According to some embodiments, the co-culturing step may be carried out with an average or set light intensity of Photosynthetic Active Radiation (PAR) in a range from about 0 μ/m²·s to about 200 μ/m²·s, 20 μ/m²·s to about 200 μ/m²·s, 20 μ/m²·s to about 180 μ/m²·s, 30 μ/m²·s to about 180 μ/m²·s, 30 μ/m²·s to about 150 μ/m²·s, 30 μ/m²·s to about 120 μ/m²·s, 60 μ/m²·s to about 120 μ/m²·s, 70 μ/m²·s to about 110 μ/m²·s, or 80 μ/m²·s to about 100 μ/m²·s, According to some embodiments, the co-culturing step may be carried out with an average or set light intensity of Photosynthetic Active Radiation (PAR) at about 0 μ/m²·s, about 10 μ/m²·s, about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130μ/m²·s, about 140μ/m²·s, about 150μ/m²·s, about 160μ/m²·s, about 170μ/m²·s, about 180 μ/m²·s, about 190 μ/m²·s, or about 200 μ/m²·s, According to some embodiments, different amounts of light and dark cycles may be used during the co-culture step, which may comprise a presence of lighting for a length of time between about 0 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the co-culture step may be about 0 hours of light and about 24 hours of dark, about 1 hour of light and about 23 hours of dark, about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

### D. Bud Induction and Extended Bud Induction

According to present embodiments, a monocot seed embryo explant that has been transformed or edited by introducing a heterologous polynucleotide molecule into at least one cell of the embryo explant may be cultured in contact with at least a first bud induction medium comprising an auxin and a cytokinin. The monocot seed embryo explant may have been inoculated with an inoculation medium comprising a Rhizobiales or Agrobacterium that comprises the heterologous polynucleotide molecule, and the monocot seed embryo explant may also have been co-cultured in contact with a co-culture medium, prior to the bud induction step.

As provided herein, the monocot seed embryo explant may be further cultured in contact with a second or extended bud induction medium comprising an auxin and a cytokinin and then cultured in contact with a regeneration medium to produce a genetically modified plant or plant part. In some embodiments, the methods described herein comprise culturing the monocot seed embryo explant in contact with a second bud induction medium after the monocot seed embryo explant is cultured in contact with the bud induction medium (or first bud induction medium) and before regenerating the genetically modified monocot plant or plant part from the cultured monocot seed embryo explant in contact with a regeneration medium. In another embodiment, the (first) bud induction medium and/or the second (or extended) bud induction medium may each comprise a high cytokinin to auxin ratio.

According to present embodiments, the bud induction medium (or first bud induction medium) and the second bud induction medium (or extended bud induction medium) may each comprise a variety of standard culture media or solution ingredients or components, such as for example, basal salts, macronutrients, micronutrients, sugars, antibiotics and/or vitamins. The bud induction medium (or first bud induction medium) and the second bud induction medium (or extended bud induction medium) may each comprise an auxin and a cytokinin. The bud induction medium (or first bud induction medium) and the second bud induction medium (or extended bud induction medium) may each comprise one or more selection agent(s), although according to many embodiments, a selection agent is absent in the first bud induction medium. The absence of the selection agent in the first bud induction medium may allow the first bud induction medium to function as a delay medium. The identity of the selection agent will typically depend on the selectable marker gene present in the heterologous polynucleotide molecule introduced into the monocot seed embryo explant. The bud induction medium (or first bud induction medium) and/or the second bud induction medium (or extended bud induction medium) may each be a solid, semi-solid or liquid medium, although each of these media may typically be a solid medium. A solid medium may comprise a gelling or polymeric agent or ingredient, such as agarose, etc., that can solidify and form the solid medium.

As used herein a “high cytokinin to auxin ratio” generally refers to a condition wherein the level of cytokinin activity is relatively high in comparison to the level of auxin activity present in the medium, which may typically be a cytokinin:auxin ratio of at least about 1:1 or higher in term of weight/vol; provided, however, that the exact cytokinin:auxin ratio will depend on the exact chemical identities of the auxin and cytokinin since different auxins and cytokinins can have different activities and/or modes of action as known in the art. The levels of cytokinin and auxin in a medium having a high cytokinin to auxin ratio may be present in the medium (measured in terms of weight/volume), for example, at a ratio of about 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, or 15:1, including all ranges derivable therebetween.

The levels of cytokinin and auxin in a culture medium having a high cytokinin to auxin ratio may be, for example, greater than or equal to about 1:1 or at least about 1:1 or higher, greater than or equal to about 1.5:1 or at least about 1.5:1 or higher, greater than or equal to about 2:1 or at least about 2:1 or higher, greater than or equal to about 2.5:1 or at least about 2.5:1 or higher, greater than or equal to about 3:1 or at least about 3:1 or higher, greater than or equal to about 3.5:1 or at least about 3.5:1 or higher, greater than or equal to about 4:1 or at least about 4:1 or higher, greater than or equal to about 4.5:1 or at least about 4.5:1 or higher, greater than or equal to about 5:1 or at least about 5:1 or higher, greater than or equal to about 5.5:1 or at least about 5.5:1 or higher, greater than or equal to about 6:1 or at least about 6:1 or higher, greater than or equal to about 6.5:1 or at least about 6.5:1 or higher, greater than or equal to about 7:1 or at least about 7:1 or higher, greater than or equal to about 7.5:1 or at least about 7.5:1 or higher, greater than or equal to about 8:1 or at least about 8:1 or higher, greater than or equal to about 8.5:1 or at least about 8.5:1 or higher, greater than or equal to about 9:1 or at least about 9:1 or higher, greater than or equal to about 9.5:1 or at least about 9.5:1 or higher, greater than or equal to about 10:1 or at least about 10:1 or higher, greater than or equal to about 10.5:1 or at least about 10.5:1 or higher, greater than or equal to about 11:1 or at least about 11:1 or higher, greater than or equal to about 11.5:1 or at least about 11.5:1 or higher, or greater than or equal to about 12:1 or at least about 12:1 or higher, including all ranges derivable therebetween.

The levels of cytokinin and auxin in a culture medium having a high cytokinin to auxin ratio may be, for example, in a range between about 1:1 and about 12:1, about 2:1 and about 12:1, about 4:1 and about 12:1, about 6:1 and about 12:1, about 8:1 and about 12:1, about 1:1 and about 10:1, about 2:1 and about 10:1, about 4:1 and about 10:1, about 6:1 and about 10:1, about 8:1 and about 10:1, about 1:1 and about 8:1, about 2:1 and about 8:1, about 4:1 and about 8:1, about 6:1 and about 8:1, about 1:1 and about 6:1, about 2:1 and about 6:1, about 4:1 and about 6:1, about 1:1 and about 5:1, about 2:1 and about 5:1, about 3:1 and about 5:1, about 1:1 and about 4:1, about 2:1 and about 4:1, about 3:1 and about 4:1, about 1:1 and about 3:1, or about 1:1 and about 2:1, including all ranges derivable therebetween.

Non-limiting examples of cytokinins that may be used in the accordance with the present inventions may include, but are not limited to: 6-benzylaminopurine (BAP), thidiazuron (TDZ), N-(2-chloro-4-pyridyl)-N-phenylurea (4-CPPU), kinetin, zeatin, diphenyl urea (DPU), 6-(gamma,gamma-dimethylallylamino) purine (2iP), and 6-(3-hydroxybenzylamino) purine (meta-topolin). Auxins which may be used in accordance with the present inventions may include, but are not limited to: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba).

According to present embodiments, the bud induction medium (or first bud induction medium) may comprise the same or different auxin and/or the same or different cytokinin than the second bud induction medium (or extended bud induction medium). The bud induction medium (or first bud induction medium) may comprise a first auxin and a first cytokinin, and the second bud induction medium or extended bud induction medium may comprise the first auxin or a second auxin and the first cytokinin or a second cytokinin. According to some embodiments, the second bud induction medium (or extended bud induction medium) may comprise the same auxin or a different auxin as the bud induction medium (or the first bud induction medium). According to some embodiments, the second bud induction (or extended bud induction medium) may comprise the same cytokinin or a different cytokinin as the bud induction medium (or first bud induction medium).

According to present embodiments, the concentration of the cytokinin (or two or more cytokinins) in the first bud induction medium and/or the second (or extended) bud induction medium is in a range from about 0.1 mg/L to about 100.0 mg/L, 1 mg/L to about 90.0 mg/L, 1 mg/L to about 80.0 mg/L, 1 mg/L to about 75.0 mg/L, 2 mg/L to about 90.0 mg/L, 2 mg/L to about 80.0 mg/L, 2 mg/L to about 75.0 mg/L, 5 mg/L to about 90.0 mg/L, 5 mg/L to about 80.0 mg/L, 5 mg/L to about 75.0 mg/L, 5 mg/L to about 70.0 mg/L, 10 mg/L to about 90.0 mg/L, 10 mg/L to about 80.0 mg/L, 10 mg/L to about 75.0 mg/L, 10 mg/L to about 70.0 mg/L, 15 mg/L to about 90.0 mg/L, 15 mg/L to about 80.0 mg/L, 15 mg/L to about 75.0 mg/L, 15 mg/L to about 70.0 mg/L, 20 mg/L to about 90.0 mg/L, 20 mg/L to about 80.0 mg/L, 20 mg/L to about 75.0 mg/L, 20 mg/L to about 70.0 mg/L, 20 mg/L to about 60.0 mg/L, 30 mg/L to about 90.0 mg/L, 30 mg/L to about 80.0 mg/L, 30 mg/L to about 75.0 mg/L, 30 mg/L to about 70.0 mg/L, 30 mg/L to about 60.0 mg/L, 40 mg/L to about 90.0 mg/L, 40 mg/L to about 80.0 mg/L, 40 mg/L to about 75.0 mg/L, 40 mg/L to about 70.0 mg/L, 40 mg/L to about 60.0 mg/L, about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 0.1 mg/L to about 15.0 mg/L, about 0.1 mg/L to about 12.5 mg/L, about 0.1 mg/L to about 10.0 mg/L, about 0.1 mg/L to about 7.5 mg/L, about 0.1 mg/L to about 5.0 mg/L, about 0.1 mg/L to about 4.0 mg/L, about 0.1 mg/L to about 3.0 mg/L, about 0.2 mg/L to about 15.0 mg/L, about 0.2 mg/L to about 12.5 mg/L, about 0.2 mg/L to about 10.0 mg/L, about 0.2 mg/L to about 7.5 mg/L, about 0.2 mg/L to about 5.0 mg/L, about 0.2 mg/L to about 4.0 mg/L, about 0.2 mg/L to about 3.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 5.0 mg/L, about 0.5 mg/L to about 4.0 mg/L, about 0.5 mg/L to about 3.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 5.0 mg/L, about 1.0 mg/L to about 5.0 mg/L, about 1.0 mg/L to about 4.0 mg/L, or about 1.0 mg/L to about 3.0 mg/L, including all ranges derivable therebetween. In some embodiments, the concentration of the cytokinin in the first bud induction medium or the second bud induction medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, about 25.0 mg/L, about 30 mg/L, about 40 mg/L, about 50 mg/L, about 60 mg/L, about 70 mg/L, about 75 mg/L, about 80 mg/L, about 90 mg/L, or about 100 mg/L, including all ranges derivable therebetween. The cytokinin in the first and second bud induction media may be the same or different, and each of these bud induction media may comprise one or more cytokinins.

In some embodiments, the concentration of the auxin (or two or more auxins) the first bud induction medium and/or the second (or extended) bud induction medium is in the range from about 0.01 mg/L to about 25.0 mg/L, about 0.05 mg/L to about 25 mg/L, about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, about 9.0 mg/L to about 11.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, about 0.1 mg/L to about 15.0 mg/L, about 0.1 mg/L to about 12.5 mg/L, about 0.1 mg/L to about 10.0 mg/L, about 0.1 mg/L to about 7.5 mg/L, about 0.1 mg/L to about 5.0 mg/L, about 0.1 mg/L to about 4.0 mg/L, about 0.1 mg/L to about 3.0 mg/L, about 0.2 mg/L to about 15.0 mg/L, about 0.2 mg/L to about 12.5 mg/L, about 0.2 mg/L to about 10.0 mg/L, about 0.2 mg/L to about 7.5 mg/L, about 0.2 mg/L to about 5.0 mg/L, about 0.2 mg/L to about 4.0 mg/L, about 0.2 mg/L to about 3.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 5.0 mg/L, about 0.5 mg/L to about 4.0 mg/L, about 0.5 mg/L to about 3.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, about 0.4 mg/L to about 0.6 mg/L, about 0.05 mg/L to about 7.5 mg/L, about 0.02 mg/L to about 5 mg/L, or about 0.75 mg/L to about 2.5 mg/L, including all ranges derivable therebetween. In some embodiments, the concentration of the auxin in the first bud induction or second bud induction medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable therebetween. The auxins in the first and second bud induction media may be the same or different, and each of these media may comprise one or more auxins.

In some embodiments, the first bud induction medium and/or the second (or extended) bud induction medium may comprise a cytokinin, wherein the cytokinin is 6-benzylaminopurine (BAP). In some embodiments, the concentration of BAP in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of BAP in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise a cytokinin, wherein the cytokinin is thidiazuron (TDZ). In some embodiments, the concentration of TDZ in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of TDZ in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise a cytokinin, wherein the cytokinin is N-(2-chloro-4-pyridyl)-N-phenylurea (4-CPPU). In some embodiments, the concentration of 4-CPPU in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of 4-CPPU in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise a cytokinin, wherein the cytokinin is kinetin. In some embodiments, the concentration of kinetin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L. or about 9.0 mg/L to about 11.0 mg/L, including all ranges derivable therebetween. In some embodiments, the concentration of kinetin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise a cytokinin, wherein the cytokinin is zeatin. In some embodiments, the concentration of zeatin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of zeatin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise a cytokinin, wherein the cytokinin is 6-(gamma,gamma-dimethylallylamino) purine (2iP). In some embodiments, the concentration of 2iP in the first bud induction medium and/or the second (or extended) bud induction medium may be in the range from 5 mg/L to about 100.0 mg/L, 5 mg/L to about 90.0 mg/L, 5 mg/L to about 80.0 mg/L, 5 mg/L to about 75.0 mg/L, 5 mg/L to about 70.0 mg/L, 10 mg/L to about 100.0 mg/L, 10 mg/L to about 90.0 mg/L, 10 mg/L to about 80.0 mg/L, 10 mg/L to about 75.0 mg/L, 10 mg/L to about 70.0 mg/L, 15 mg/L to about 100.0 mg/L, 15 mg/L to about 90.0 mg/L, 15 mg/L to about 80.0 mg/L, 15 mg/L to about 75.0 mg/L, 15 mg/L to about 70.0 mg/L, 20 mg/L to about 100.0 mg/L, 20 mg/L to about 90.0 mg/L, 20 mg/L to about 80.0 mg/L, 20 mg/L to about 75.0 mg/L, 20 mg/L to about 70.0 mg/L, 20 mg/L to about 60.0 mg/L, 30 mg/L to about 100.0 mg/L, 30 mg/L to about 90.0 mg/L, 30 mg/L to about 80.0 mg/L, 30 mg/L to about 75.0 mg/L, 30 mg/L to about 70.0 mg/L, 30 mg/L to about 60.0 mg/L, 40 mg/L to about 100.0 mg/L, 40 mg/L to about 90.0 mg/L, 40 mg/L to about 80.0 mg/L, 40 mg/L to about 75.0 mg/L, 40 mg/L to about 70.0 mg/L, 40 mg/L to about 60.0 mg/L, including all ranges derivable therebetween. In some embodiments, the concentration of 2iP in the first bud induction medium and/or the second (or extended) bud induction medium may be about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, about 30 mg/L, about 40 mg/L, about 50 mg/L, about 60 mg/L, about 70 mg/L, about 75 mg/L, about 80 mg/L, about 90 mg/L, or about 100 mg/L, including all ranges derivable there between.

In some embodiments, the first bud induction medium and/or the second (or extended) bud induction medium may comprise a cytokinin, wherein the cytokinin is 6-(3-hydroxybenzylamino) purine (meta-topolin). In some embodiments, the concentration of meta-topolin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of meta-topolin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise an auxin, wherein the auxin is 2,4-dichlorophenoxy-acetic acid (2,4-D). In some embodiments, the concentration of 2,4-D in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all ranges derivable there between. In some embodiments, the concentration of 2,4-D in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is 2,4,5-trichloro-phenoxy acetic acid (2,4,5-T). In some embodiments, the concentration of 2,4,5-T in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all ranges derivable there between. In some embodiments, the concentration of 2,4,5-T in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is 4-amino-3,5,6-trichloro-picolinic acid (picloram). In some embodiments, the concentration of picloram in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of picloram in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is indole-3-acetic acid (IAA). In some embodiments, the concentration of IAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of IAA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise an auxin, wherein the auxin is indole-3-butyric acid (IBA). In some embodiments, the concentration of IBA in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of IBA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is naphthalene acetic acid (NAA). In some embodiments, the concentration of NAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 25.0 mg/L, about 0.2 mg/L to about 20.0 mg/L, about 0.2 mg/L to about 15.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 25.0 mg/L, about 3.0 mg/L to about 20.0 mg/L, about 3.0 mg/L to about 15.0 mg/L, about 3.0 mg/L to about 12.5 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 25.0 mg/L, about 4.0 mg/L to about 20.0 mg/L, about 4.0 mg/L to about 15.0 mg/L, about 4.0 mg/L to about 12.5 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, including all ranges derivable there between. In some embodiments, the concentration of NAA in the first bud induction medium and/or the second (or extended) bud induction medium may be 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, or about 20.0 mg/L, including all ranges derivable there between.

In some embodiments, the first bud induction medium and/or the second (or extended) bud induction medium may comprise an auxin, wherein the auxin is 2,3,5-triiodobenzoic acid (TIBA). In some embodiments, the concentration of TIBA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 25.0 mg/L, about 0.2 mg/L to about 20.0 mg/L, about 0.2 mg/L to about 15.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 25.0 mg/L, about 3.0 mg/L to about 20.0 mg/L, about 3.0 mg/L to about 15.0 mg/L, about 3.0 mg/L to about 12.5 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 25.0 mg/L, about 4.0 mg/L to about 20.0 mg/L, about 4.0 mg/L to about 15.0 mg/L, about 4.0 mg/L to about 12.5 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, including all ranges derivable there between. In some embodiments, the concentration of TIBA in the first bud induction medium and/or the second (or extended) bud induction medium may be 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, or about 20.0 mg/L, including all ranges derivable there between.

In some embodiments, the first bud induction medium and/or the second (or extended) bud induction medium may comprise an auxin, wherein the auxin is phenylacetic acid (PAA). In some embodiments, the concentration of PAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of PAA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise an auxin, wherein the auxin is 3,6-dichloro-2-methoxy-benzoic acid (dicamba). In some embodiments, the concentration of dicamba in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of dicamba in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

According to some embodiments, the first bud induction medium comprises a first auxin and a first cytokinin, wherein the first auxin is 2,4-dichlorophenoxy-acetic acid (2,4-D) and the first cytokinin is 6-benzylaminopurine (BAP). According to these embodiments, the concentration of 2,4-D in the first bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all ranges derivable there between. According to these embodiments, the concentration of 6-benzylaminopurine (BAP) in the first bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween.

According to some embodiments, the second (or extended) bud induction medium comprises a second auxin and a second cytokinin, wherein the second auxin is 4-amino-3,5,6-trichloro-picolinic acid (picloram) and the second cytokinin is thidiazuron (TDZ). According to these embodiments, the concentration of picloram in the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. According to these embodiments, the concentration of TDZ in the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between.

According to embodiments of the present inventions, the monocot seed embryo explant(s) may be cultured in contact with the first bud induction medium for about 2 days to about 14 days, about 4 days to about 12 days, about 5 days to about 10 days, or about 6 days to about 8 days, including all ranges derivable therebetween. According to some embodiments, the monocot seed embryo explant(s) is/are cultured in contact with the first bud induction medium for about 2 days, about 3 days, about 4 days, about 5 days, about 6 days, about 7 days (or about 1 week), about 8 days, about 9 days, about 10 days, about 11 days, about 12 days, about 13 days, or about 14 days (or about 2 weeks), including all ranges derivable therebetween. In some embodiments, the monocot seed embryo explant(s) may be cultured in contact with the first bud induction medium at a temperature in a range from about 20° C. to about 30° C., about 22° C. to about 28° C., about 25° C. to about 30° C., about 25° C. to about 29° C., or about 25° C. to about 28° C., including all ranges derivable therebetween. According to some embodiments, the monocot seed embryo explant(s) may be cultured in contact with the first bud induction medium at a temperature of about 20° C., about 21° C., about 22° C., about 23° C., about 24° C., about 25° C., about 26° C., about 27° C., about 28° C., about 29° C., or about 30° C., including all ranges derivable therebetween. According to an aspect of the present disclosure, the monocot seed embryo explant(s) in contact with the first bud induction medium at elevated temperature, which may be in a range from about 30° C. to about 40° C., about 30° C. to about 38° C., about 30° C. to about 36° C., about 30° C. to about 35° C., about 31° C. to about 40° C., about 31° C. to about 38° C., about 31° C. to about 36° C., about 31° C. to about 35° C., about 32° C. to about 40° C., about 32° C. to about 38° C., about 32° C. to about 36° C., about 32° C. to about 35° C., about 33° C. to about 40° C., about 33° C. to about 38° C., about 33° C. to about 36° C., or about 33° C. to about 35° C., including all ranges derivable therebetween. According to some embodiments, the monocot seed embryo explant(s) may be cultured in contact with the first bud induction medium at an elevated temperature of about 30° C., about 31° C., about 32° C., about 33° C., about 34° C., about 35° C., about 36° C., about 37° C., about 38° C., about 39° C., or about 40° C., including all ranges derivable therebetween. A selection agent may generally be absent from the first bud induction medium, but the first bud induction medium may alternatively comprise a selection agent.

In another aspect, culturing monocot seed embryo explants in contact with the first bud induction medium at an elevated temperature, for example a temperature in a range from about 30° C. to about 40° C., for about one week may improve transformation by at least about 0.2-fold, 0.4-fold, 0.6-fold, 0.8-fold, 1-fold, 2-fold, 3-fold, 4-fold, 5-fold, 6-fold, 7-fold, 8-fold, 9-fold, 10-fold, 15-fold, 20-fold, 30-fold, or 40-fold, including all ranges derivable therebetween, as compared to culturing the explants in contact with the first bud induction medium at a lower temperature, for example at a temperature in a range from about 20° C. to about 30° C., during the first bud induction step.

The first bud induction step may also be carried out under a variety of lighting conditions. While some degree of lighting may generally be used, all or part of the first bud induction step may alternatively be performed in the dark. According to some embodiments, the first bud induction step may be carried out with an average or set light intensity of Photosynthetic Active Radiation (PAR) in a range from about 0 μ/m²·s to about 200 μ/m²·s, 20 μ/m²·s to about 200 μ/m²·s, 20 μ/m²·s to about 180 μ/m²·s, 30 μ/m²·s to about 180 μ/m²·s, 50 μ/m²·s to about 180 μ/m²·s, 50 μ/m²·s to about 150 μ/m²·s, 60 μ/m²·s to about 150 μ/m²·s, 70 μ/m²·s to about 140 μ/m²·s, 80 μ/m²·s to about 130 μ/m²·s, or 90 μ/m²·s to about 120 μ/m²·s, According to some embodiments, the first bud induction step may be carried out with an average or set light intensity of Photosynthetic Active Radiation (PAR) at about 0 μ/m²·s, about 10 μ/m²·s, about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130 μ/m²·s, about 140 μ/m²·s, about 150 μ/m²·s, about 160 μ/m²·s, about 170 μ/m²·s, about 180 μ/m²·s, about 190 μ/m²·s, or about 200 μ/m²·s, According to some embodiments, different amounts of light and dark cycles may be used during the first bud induction step, which may comprise a presence of lighting for a length of time between about 0 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

In some embodiments, culturing the monocot seed embryo explant with a first bud induction medium comprises culturing the explant with a photoperiod of about 16 hours light/8 hours dark. In still further embodiments, the photoperiod is about 16-hour light/8-hour dark for about 6-24 days. In some embodiments the light intensity during the culturing is about 90 μMol m⁻² sec⁻¹ to about 160 μMol m⁻² sec⁻¹ at a wavelength of about 400-700 nm. In particular embodiments, the light intensity is about 90 μMol m²² sec⁻¹ at a wavelength of about 400-700 nm.

According to some embodiments, the amounts of light and dark cycles during the first bud induction step may be about 0 hours of light and about 24 hours of dark, about 1 hour of light and about 23 hours of dark, about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

According to embodiments of the present inventions, the monocot seed embryo explant(s) may be cultured in contact with the second (or extended) bud induction medium for about 4 days to about 28 days, about 4 days to about 25 days, about 4 days to about 21 days, about 5 days to about 25 days, about 5 days to about 23 days, about 7 days to about 21 days, about 5 days to about 15 days, about 7 days to about 14 days, about 12 days to about 23 days, or about 14 days to about 21 days, including all ranges derivable therebetween. According to some embodiments, the monocot seed embryo explant(s) is/are cultured in contact with the second (or extended) bud induction medium for about 4 days, about 5 days, about 6 days, about 7 days (or about 1 week), about 8 days, about 9 days, about 10 days, about 11 days, about 12 days, about 13 days, about 14 days (or about 2 weeks), about 15 days, about 16 days, about 17 days, about 18 days, about 19 days, about 20 days, about 21 days (or about 3 weeks), about 22 days, about 23 days, about 24 days, about 25 days, about 26 days, about 27 days, or about 28 days (or about 4 weeks), including all ranges derivable therebetween. In some embodiments, the monocot seed embryo explant(s) may be cultured in contact with the second (or extended) bud induction medium at a temperature in a range from about 20° C. to about 32° C., about 20° C. to about 30° C., about 22° C. to about 28° C., about 25° C. to about 30° C., about 25° C. to about 29° C., about 26° C. to about 29° C., about 25° C. to about 28° C., or about 27° C. to about 28° C., including all ranges derivable therebetween. According to some embodiments, the monocot seed embryo explant(s) may be cultured in contact with the second (or extended) bud induction medium at a temperature of about 20° C., about 21° C., about 22° C., about 23° C., about 24° C., about 25° C., about 26° C., about 27° C., about 28° C., about 29° C., or about 30° C., including all ranges derivable therebetween. In a particular embodiment, the monocot seed embryo explant may be cultured in contact with a first bud induction medium for a time period in a range from about 2 days to about 14 days at a temperature in a range from about 20° C. to about 30° C. or at an elevated temperature in a range from about 30° C. to about 40° C., and then subsequently cultured in contact with a second (or extended) bud induction medium for a time period in a range from about 4 days to about 28 days at a temperature in a range from about 20° C. to about 32° C. The second (or extended) bud induction medium may also comprise a selection agent. In another embodiment, culturing the explant in contact with the second bud induction medium may improve transformation by at least about 0.2-fold, 0.4-fold, 0.6-fold, 0.8-fold, 1-fold, 2-fold, 3-fold, 4-fold, 5-fold, 6-fold, 7-fold, 8-fold, 9-fold, 10-fold, 15-fold, 20-fold, 30-fold, or 40-fold.

The second (or extended) bud induction step may also be carried out under a variety of lighting conditions. Some degree of lighting may generally be used during the second (or extended) bud induction step. According to some embodiments, the second (or extended) bud induction step may be carried out with an average or set light intensity of Photosynthetic Active Radiation (PAR) in a range from about 30 μ/m²·s to about 200 μ/m²·s, 30 μ/m²·s to about 180 μ/m²·s, 50 μ/m²·s to about 180 μ/m²·s, 50 μ/m²·s to about 150 μ/m²·s, 60 μ/m²·s to about 150 μ/m²·s, 70 μ/m²·s to about 140 μ/m²·s, 80 μ/m²·s to about 130 μ/m²·s, or 90 μ/m²·s to about 120 μ/m²·s, According to some embodiments, the second (or extended) bud induction step may be carried out with an average or set light intensity of Photosynthetic Active Radiation (PAR) at about 10 μ/m²·s, about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130 μ/m²·s, about 140 μ/m²·s, about 150 μ/m²·s, about 160 μ/m²·s, about 170 μ/m²·s, about 180 μ/m²·s, about 190 μ/m²·s, or about 200 μ/m²·s, According to some embodiments, different amounts of light and dark cycles may be used during the second (or extended) bud induction step, which may comprise a presence of lighting for a length of time between about 2 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the second (or extended) bud induction step may be about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

Without being bound by theory, the bud induction step(s) may cause differentiation and/or proliferation of cells of the explant to form multiple buds on the explant, which may then be regenerated into a plant. According to some preferred embodiments, the first auxin and cytokinin are different that than the second auxin and cytokinin to affect the formation of the multiple buds through somewhat activities and/or modes of action. Without being bound by theory, the first bud induction step may cause differentiation of cells of the explant into multiple buds, whereas the second (or extended) bud induction step may greater proliferation or expansion of the multiple buds to produce a more compact or solid multiple bud explant for further culturing and regeneration into a plant that may have the further benefit of reducing chimerism of the resulting genetically modified plant or plant part. According to some embodiments, culturing the monocot seed embryo explant(s) in a first bud induction medium followed by a second (or extended) bud induction medium may reduce chimerism in regenerated plants by at least 10%, at least 20%, at least 30%, at least 40%, at least 50%, at least 60%, at least 70%, at least 80%, or at least 90%, as compared to culturing the monocot seed embryo explant(s) in the first bud induction medium but without culturing in the second (or extended) bud induction medium prior to regeneration.

### E. Flotation of Cultured Embryo Explants

Cultured monocot seed embryo explant(s) can be transferred directly or indirectly from the first bud induction medium or the second (or extended) bud induction medium to a regeneration medium to regenerate or produce one or more genetically modified monocot plants or plant parts. However, many of the cultured monocot seed embryo explant(s) in contact with the regeneration medium may not regenerate into monocot plants or plant parts because the cultured monocot seed embryo explant(s) may be non-viable and/or non-regenerable. Even though some selection of explants transferred to the regeneration medium can be done based on visual observation, it is often not clear or obvious which explants are viable and regenerable based solely on their visual appearance to the observer's eye. The cultured monocot seed embryo explant(s) that do not regenerate into healthy monocot plants or plant parts are unproductive and increase costs by consuming laboratory time, space and resources. Therefore, it would be beneficial if the number and proportion of these unproductive explants could be reduced through their separation and removal prior to regeneration to conserve laboratory time, space and resources in the subsequent regeneration and controlled environment steps, especially if a similar or higher number and/or frequency of regenerated plants recovered relative to the total number cultured monocot seed embryo explant(s) present before such separation and removal could be achieved or maintained. Even if the number and/or frequency of regenerated plants recovered is reduced somewhat relative to a standard protocol, the overall cost efficiency may still be greater relative to a standard or protocol due to the greater cost savings in subsequent regeneration and controlled environment steps by removing the non-viable or non-regenerable explants. It is proposed and shown herein that the proportion of cultured explants that are viable and regenerable can be preferentially enriched by their greater buoyancy and flotation in water or aqueous medium compared to inviable and/or non-regenerable explants.

According to embodiments of the present disclosure, cultured monocot seed embryo explant(s) from the first bud induction medium or the second (or extended) bud induction medium can be subjected to an optional flotation step prior to regeneration by placing the cultured monocot seed embryo explant(s) in contact with a flotation medium in a flotation container or vessel to allow the cultured monocot seed embryo explant(s) to separate into top and bottom layers, portions or fractions, collecting the top fraction or portion of cultured explants floating at or near the top surface of the flotation medium, and then transferring the top fraction or portion of cultured explants to a regeneration medium. The bottom fraction or portion of cultured explants sinks below the top surface of the flotation medium and toward the bottom of the flotation medium and the flotation container or vessel and thus becomes separated from the top fraction or portion of cultured explants floating at or near the top surface of the flotation medium. The bottom fraction or portion of cultured explants can be discarded or used as a control for the top fraction or portion of cultured explants in subsequent regeneration and controlled environment steps. As shown herein, the top fraction or portion is enriched with viable and regenerable explants, whereas the bottom fraction or portion has a much greater number and proportion of non-viable and/or non-regenerable explants.

According to some embodiments, a plurality of monocot seed embryo explants can be contacted with a flotation medium, after culturing the monocot seed embryo explants in contact with a first bud induction medium and/or a second (or extended) bud induction medium, to separate the plurality of monocot seed embryo explants into a first portion of the plurality of monocot seed embryo explants present within a top region of the flotation medium and a second portion of the plurality of monocot seed embryo explants present within a bottom region of the flotation medium, and all or part of the first portion of the plurality of monocot seed embryo explants can then be collected from a top region of the flotation medium to select, separate and remove the first portion of the plurality of monocot seed embryo explants from the flotation container or vessel. As a result, the collected first portion can be selected, separated and removed from the second portion of the plurality of monocot seed embryo explants present within a bottom region of the flotation medium, and the second portion can be left behind in the flotation container or vessel or separately collected for use, such as for use as a control. The explants in the top portion can be collected using any suitable means, such as by using a mesh, strainer, spoon, scoop or other transfer tool, or by decanting or pouring the top portion into another container or transfer tool. Given that the first and second portions of the plurality of monocot seed embryo explants are separated within the flotation medium, all or part of the first portion or the second portion can be separately collected and transferred without the other portion. Thus, all or part of the first portion of monocot seed embryo explants can be selectively collected and transferred (directly or indirectly) to a regeneration medium without the second portion of monocot seed embryo explants.

As used herein in reference to a first portion or a second portion of monocot seed embryo explants in the flotation protocol, “selectively collecting” means to collect either all or part of the first portion or all or part of the second portion of monocot seed embryo explants, as the case may be, without the other portion of monocot seed embryo explants. For example, “selectively collecting the first portion” means to collect all or part of the first portion of monocot seed embryo explants without the second portion of monocot seed embryo explants (i.e., the second portion of monocot seed embryo explants are not collected and remain in the flotation container or vessel if not separately collected). It is specified that all or part of the first or second portion of the monocot seed embryo explants may be selectively collected since not all explants or not every explant of the first portion or the second portion may be selectively collected, either intentionally or otherwise. The terms “collected first portion” and “collected second portion” each refer to the explants of the first portion or the second portion, respectively, that are selectively collected in actuality, and which may be further transferred to a regeneration medium.

The top region refers to a space or volume of the flotation medium at or near the top surface of the flotation medium where the first portion of the plurality of monocot seed embryo explants localizes and is present, and the bottom region refers to a space or volume of the flotation medium below the top region where the second portion of the plurality of monocot seed embryo explants localizes and is present. Preferably, the bottom region is separated from the top region by an average gap distance or spacing within the flotation medium to allow for separate collection of the first and/or second portion(s) of cultured monocot seed embryo explants. In an aspect, the average gap distance or spacing between the top region and the bottom region is in a range from about 0.25 inches to about 12 inches, from about 0.5 inches to about 10 inches, from about 0.5 inches to about 5 inches, from about 1 inch to about 5 inches, from about 1.5 inches to about 5 inches, from about 2 inches to about 5 inches, from about 1 inch to about 4 inches, from about 1 inch to about 3 inches, or from about 1 inch to about 2 inches. It is understood by one skilled in the art that measurements in inches can be converted into metric centimeters.

In an aspect, the flotation medium comprises, consists or consists essentially of water. In an aspect, the flotation medium comprises, consists or consists essentially of an aqueous solution comprising at least one salt. In some embodiments, the flotation medium comprises an antimicrobial agent. The flotation medium will generally be contained within a flotation vessel or container while in contact with the monocot seed embryo explants. The flotation vessel or container may have one or more side wall(s) and a bottom wall and may optionally have a top cover or lid that may be removable or able to be opened and closed. The interior of the flotation vessel or container may have one of a variety of shapes in horizontal cross-sectional area with the side wall(s), such as a rectangle, square, triangle, circle, oval, polygon, etc., or any other regular or irregular shape. The depth of the flotation medium in the flotation vessel or container can vary as long as the flotation medium in the flotation vessel or container is deep enough to allow for separation of the top and bottom portions or fractions of the plurality of monocot seed embryo explants into two distinct and separated layers, portions or fractions with a gap distance or spacing. According to some embodiments, the depth of the flotation medium in the flotation vessel or container is in a range from about 0.5 inches to about 24 inches, from about 1 inch to about 10 inches, from about 1 inch to about 5 inches, from about 1 inch to about 4 inches, from about 1.5 inches to about 5 inches, from about 2 inches to about 5 inches, from about 2 inches to about 4 inches, or from about 2 inches to about 3 inches. It is understood by one skilled in the art that measurements in inches can be converted into metric centimeters.

In an aspect of the present disclosure, the monocot seed embryo explants may be physically agitated while in contact with the flotation medium to help the flotation medium have greater contact with the surfaces of the monocot seed embryo explants and to encourage greater separation of the two fractions, portions or layers. Such physical agitation may include, for example, shaking or rocking of the flotation vessel or container and flotation medium, such as in a linear or circular motion or in any other direction or type of motion. Such physical agitation may also include mixing or stirring of the explants and flotation medium, such as by using a utensil, rod, stir bar, etc., or vibrating or vortexing of the explants and flotation medium. According to some embodiments, the monocot seed embryo explants and flotation medium may be sonicated while the monocot seed embryo explants are in contact with the flotation medium.

In an aspect of the present disclosure, the plurality of monocot seed embryo explants is contacted with the flotation medium in the flotation vessel at a sufficient density to process a larger number of explants to be productive, but not an excessive density that would overwhelm the flotation medium relative to the top surface area of the flotation medium and interfere with the clear separation of the two layers, portions or fractions. It is proposed that the capacity of the flotation medium in terms of the number of explants that can be cleanly or clearly separated into the top and bottom fractions or portions is dependent or proportional to the top surface area and depth of the flotation medium to allow for sufficient and effective spreading and separation of the monocot seed embryo explants withing the flotation medium in the flotation vessel or container. According to some embodiments, the plurality of monocot seed embryo explants is contacted with the flotation medium in the flotation vessel at a density relative to the surface area of the upper surface of the flotation medium in the flotation vessel in a range from about 1 to about 100 monocot seed embryo explants per square inch, from about 5 to about 50 monocot seed embryo explants per square inch, from about 10 to about 50 monocot seed embryo explants per square inch, from about 15 to about 40 monocot seed embryo explants per square inch, from about 15 to about 30 monocot seed embryo explants per square inch, from about 20 to about 30 monocot seed embryo explants per square inch, or from about 25 to about 30 monocot seed embryo explants per square inch. It is understood by one skilled in the art that measurements in square inches can be converted into square centimeters.

In an aspect of the present disclosure, the plurality of monocot seed embryo explants is contacted with the flotation medium in the flotation vessel for a sufficient amount of time to allow for the plurality of monocot seed embryo explants to contact the flotation medium and become separated into the into the top and bottom fractions or portions. According to some embodiments, the plurality of monocot seed embryo explants is contacted with the flotation medium for a period of time in a range from about 5 seconds to about 12 hours, from about 10 seconds to about 6 hours, from about 10 seconds to about 3 hours, from about 10 seconds to about 1 hour, from about 10 seconds to about 30 minutes, from about 10 seconds to about 10 minutes, from about 10 seconds to about 5 minutes, from about 15 seconds to about 2 minutes, or from about 15 seconds to about 1 minute.

In an aspect of the present disclosure, all or part of the first portion of the plurality of monocot seed embryo explants collected from the top region of the flotation medium, and separated from the second portion of the plurality of monocot seed embryo explants present within the bottom portion of the flotation medium, may be transferred to a regeneration medium to regenerate one or more plants or plant parts, which may be genetically modified plants or plant parts as provided herein. According to some embodiments, the first portion of the plurality of monocot seed embryo explants are transferred directly from the flotation medium to the regeneration medium. According to some embodiments, the first portion of the plurality of monocot seed embryo explants are transferred indirectly from the flotation medium to the regeneration medium through one or more intermediate steps, washes, rinses or transfers. According to some embodiments, the first portion of the plurality of monocot seed embryo explants is blotted with paper or other absorbent material or surface prior to being transferred to the regeneration medium. According to preset embodiments, the proportion or percentage of cultured monocot seed embryo explants in the top portion or fraction that are viable and regenerable into monocot plants or plant parts is higher or increased relative to the proportion or percentage of cultured monocot seed embryo explants from the bud induction and/or second (or extended) bud induction steps that are viable and regenerable into monocot plants or plant parts according to the standard or control protocol without the flotation step. As a result, a similar or at least sufficient regeneration and/or plugging frequency can be obtained with the cultured explants in the top portion or region of the flotation medium using the flotation protocol, as compared to the total explants from the bud induction and/or second (or extended) bud induction steps using the standard or control protocol without the flotation step.

As used herein unless otherwise defined, the regeneration frequency (%) is defined as the number of regenerated plants for a given explant fraction or portion of monocot seed embryo explants, or for a total number or subset of explants in a given protocol, from a population or plurality of monocot seed embryo explants divided by the total number of explants in the population or plurality, multiplied by 100. As used herein unless otherwise defined, the plugging frequency (%) has a similar meaning herein but is defined as the number of regenerated plants that are sufficiently healthy to be selected for plugging into a growth medium or soil for a given explant fraction or portion of monocot seed embryo explants, or for a total number or subset of explants in a given protocol, from a population or plurality of monocot seed embryo explants divided by the total number of explants in the population or plurality, multiplied by 100. As used herein, the population or plurality of monocot seed embryo explants refers to the monocot seed embryo explants that are initially used in the method, or if specified, the number explants in the population or plurality of monocot seed embryo explants that are inoculated or subjected to transformation. For example, the plugging and regeneration frequencies in the Examples below are relative to the total number of inoculated explants in the experiment. If stated, the regeneration or plugging frequency may alternatively be defined as the number of plugged or regenerated plants for a given explant fraction or portion of explants, or for a total number or subset of explants in a given protocol, divided by either (i) the total number of monocot seed embryo explants from the bud induction or second (or extended) bud induction step in the protocol and experiment that are transferred to the flotation medium, or (ii) the number of monocot seed embryo explants in the relevant fraction or portion of monocot seed embryo explants that are advanced to the regeneration step (i.e., transferred to the regeneration medium).

Given that a similar or at least sufficient (if only moderately reduced) regeneration or plugging frequency can be obtained using either the top fraction from the flotation protocol or the total number of explants cultured according to the control or standard protocol, the flotation protocol can provide substantial laboratory, growth chamber and/or controlled environment time, space and cost savings due to the fewer number of explants from the bud induction or second (or extended) bud induction steps that are advanced to regeneration media and subsequent controlled environment steps. Indeed, as shown in Example 19 below, explants collected from the bottom layer or region were shown to have a very low plugging (and regeneration) frequency and would thus provide little or no benefit if advanced to regeneration media but would require substantially increased laboratory, growth chamber and/or controlled environment space to culture and grow the additional explants. Therefore, a substantial time, space and resource savings can be achieved by removing and discarding the bottom layer or portion of explants. Since the bottom layer or portion of explants can represent up to, or more than, 40% of the total number of explants (see, e.g., Table 32 in the Examples below), their removal from the subsequent culturing, growth chamber and controlled environment steps can provide a substantial amount of space and cost savings that may be proportional to the number of explants in the bottom layer. In an aspect, the number of monocot seed embryo explants in the first portion or collected first portion may be less than the number of monocot seed embryo explants in the plurality of monocot seed embryo explants used initially or in the prior bud induction step(s). According to some embodiments, the number of monocot seed embryo explants in the first portion or collected first portion is at least 5% less, at least 10% less, at least 15% less, at least 20% less, at least 25% less, at least 30% less, at least 35% less, at least 40% less, at least 45% less, or at least 50% less than the number of monocot seed embryo explants in the plurality of monocot seed embryo explants. Reduction in the number of explants advanced to regeneration and subsequent steps not only reduces the amount of laboratory space needed, but also reduces the amount of time required for laboratory personnel to prepare and transfer the cultured explants to regeneration media and perform subsequent growth chamber, controlled environment and screening steps. As used herein, a “moderately reduced” plugging or regeneration frequency means only a small reduction in the plugging or regeneration frequency, such as a reduction of about 30% or less, about 25% or less, about 20% or less, about 15% or less, about 10% or less, or about 5% or less.

According to an aspect of the present disclosure, the first portion of the plurality of monocot seed embryo explants in the flotation medium has a higher plugging and/or regeneration frequency than the second portion of the plurality of monocot seed embryo explants in the flotation medium. According to some embodiments, the plugging and/or regeneration frequency of the first portion of the plurality of monocot seed embryo explants is at least two-fold, at least three-fold, at least four-fold, at least five-fold, at least ten-fold, at least fifteen-fold, at least twenty-fold, at least twenty five-fold, at least thirty-fold, at least forty-fold, at least fifty-fold, at least seventy five-fold, or at least one hundred-fold higher or greater than the plugging or regeneration frequency of the second portion of the plurality of monocot seed embryo explants.

According to an aspect of the present disclosure, the first portion of the plurality of monocot seed embryo explants has a same or similar (or only a moderately reduced) plugging and/or regeneration frequency as a control population of monocot seed embryo explants, wherein the control population of monocot seed embryo explants is cultured using a control protocol in which the control population of monocot seed embryo explants is not contacted with a flotation medium to separate the control population of monocot seed embryo explants into top and bottom portions prior to regeneration of one or more plants from the control population. According to some of these embodiments, the number of monocot seed embryo explants in the first portion is less than the number of monocot seed embryo explants in the control population of monocot seed embryo explants, wherein the first portion has a same or similar (or only a moderately reduced) regeneration or plugging frequency as the control population. According to some of these embodiments, the number of monocot seed embryo explants in the first portion is at least 5% less, at least 10% less, at least 15% less, at least 20% less, at least 25% less, at least 30% less, at least 35% less, at least 40% less, at least 45% less, or at least 50% less than the number of monocot seed embryo explants in the control population of monocot seed embryo explants, wherein the first portion has a same or similar (or only a moderately reduced) regeneration or plugging frequency as the control population.

According to an aspect of the present disclosure, a subset of the cultured monocot seed embryo explants from a bud induction or a second or extended bud induction step can be contacted with a flotation medium, and all or part of a first portion of the subset of monocot seed embryo explants can be collected from a top region of the flotation medium to separate and remove the first portion of the subset of monocot seed embryo explants from the flotation vessel and a second portion of the subset of monocot seed embryo explants present within a bottom region of the flotation medium. According to some embodiments, a plurality of monocot seed embryo explants, which may be from a bud induction or a second or extended bud induction step, can be divided into a subset (or first subset) of monocot seed embryo explants and a second subset of monocot seed embryo explants, and the second subset of monocot seed embryo explants can be cultured in contact with a regeneration medium using a control protocol in which the second subset of monocot seed embryo explants is not contacted with a flotation medium to separate the second subset of monocot seed embryo explants into top and bottom portions prior to the regeneration step. According to some embodiments, the subset (or first subset) of the cultured monocot seed embryo explants can be contacted with a flotation medium, and all or part of a first portion of the subset of monocot seed embryo explants can be collected from a top region of the flotation medium to separate and remove the collected first portion of the subset of monocot seed embryo explants from the flotation vessel and a second portion of the subset of monocot seed embryo explants present within a bottom region of the flotation medium. According to some embodiments, the first portion from the subset (or first subset) of the plurality of monocot seed embryo explants has a same or similar (or only a moderately reduced) plugging or regeneration frequency as the second subset of the plurality of monocot seed embryo explants cultured using the control protocol. According to some embodiments, the number of monocot seed embryo explants in the subset may be approximately the same as the number of monocot seed embryo explants in the second subset. According to some embodiments, the number of monocot seed embryo explants in the first portion of monocot seed embryo explants from the subset (or first subset) is less than the number of monocot seed embryo explants in the second subset of monocot seed embryo explants, wherein the number of monocot seed embryo explants in the subset (or first subset) and the second subset are about the same. According to some embodiments, the number of monocot seed embryo explants in the first portion of monocot seed embryo explants is at least 5% less, at least 10% less, at least 15% less, at least 20% less, at least 25% less, at least 30% less, at least 35% less, at least 40% less, at least 45% less, or at least 50% less than the number of monocot seed embryo explants in the second subset of monocot seed embryo explants, wherein the number of monocot seed embryo explants in the subset (or first subset) and the second subset are about the same.

### F. Regeneration of Transformed Plants

In another aspect of the present disclosure, a genetically modified monocot plant or plant part is regenerated from a cultured monocot seed embryo explant(s) in contact with a regeneration medium. According to present embodiments, a regeneration medium may comprise a variety of standard culture media or solution ingredients or components, such as for example, basal salts, macronutrients, micronutrients, sugars, antibiotics and/or vitamins. The regeneration medium may generally not comprise an auxin or a cytokinin, although an auxin and/or a cytokinin may alternatively be present. The regeneration medium may typically comprise one or more selection agent(s). The regeneration medium may be a solid, semi-solid or liquid medium, although a regeneration media may typically be a solid medium. A solid medium may comprise a gelling or polymeric agent or ingredient, such as agarose, etc., that can solidify and form the solid medium. As used herein, the term “regeneration” refers to the process of growing a plant from one or more plant cells or tissues of an explant, and the term “regeneration medium” refers to a plant tissue culture medium formulated for regeneration of a plant from an explant. In some embodiments, regeneration or a regeneration step may refer to one or more regeneration step(s) that may involve culturing an explant or cultured explant in two or more regeneration media, which may be the same or different regeneration medium/media, such as by subculturing or transferring the explant from a first regeneration medium to a second regeneration medium, and possibly to a third regeneration medium, and so on.

According to many embodiments, the regeneration medium comprises a low salt concentration. As used herein “low salt concentration” refers to a medium comprising total salt concentration that is less than or equal to about 2800 mg/L. As used herein, a “salt” has a commonly understood meaning in the field of chemistry and refers to an ionic chemical compound, or a dissolved chemical compound if present in a solution, comprising at least one cation (or base) and at least one anion (or acid). The regeneration medium may comprise, in some embodiments, a total salt concentration of less than or equal to about 3000 mg/L, 2800 mg/L, about 2700 mg/L, about 2600 mg/L, about 2500 mg/L, about 2400 mg/L, about 2300 mg/L, about 2200 mg/L, about 2100 mg/L, or about 2000 mg/L. In another embodiment, the regeneration medium may comprise a salt concentration of about 1200 mg/L to about 3000 mg/L, about 1200 mg/L to about 2800 mg/L, about 1300 mg/L to about 2700 mg/L, about 1400 mg/L to about 2600 mg/L, about 1500 mg/L to about 2500 mg/L, about 1600 mg/L to about 2400 mg/L, about 1700 mg/L to about 2400 mg/L, about 1800 mg/L to about 2400 mg/L, about 1900 mg/L to about 2400 mg/L, about 2000 mg/L to about 2400 mg/L, about 2100 mg/L to about 2400 mg/L, about 2200 mg/L to about 2400 mg/L, or about 2300 mg/L, including all ranges derivable therebetween. The total nitrogen concentration of the regeneration medium may, in some embodiments, be in a range from about 0.5 mM to about 20 mM, about 0.5 mM to about 10 mM, about 1 mM to about 20 mM, about 5 mM to about 20 mM, about 1 mM to about 15 mM, about 5 mM to about 15 mM, about 1 mM to about 10 mM, about 1 mM to about 7.5 mM, about 2.5 mM to about 7.5 mM, about 5 mM to about 10 mM, about 10 mM to about 15 mM, about 10 mM to about 20 mM, or about 15 mM to about 20 mM, including all ranges derivable therebetween. As used herein, the term “total nitrogen concentration” refers to the total concentration of nitrogen containing ions, such as nitrate and ammonium ions.

A regeneration medium for use according to the methods described herein may be described, in some embodiments, in terms of its nitrate, ammonium, potassium, or sulfate ion concentration. The nitrate ion concentration may be for example about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM, including all ranges derivable therebetween. The ammonium ion concentration may be for example about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM, including all ranges derivable therebetween. The potassium ion concentration may be for example about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM, including all ranges derivable therebetween. The sulfate ion concentration may be for example about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM, including all ranges derivable therebetween.

The regeneration medium may, in some embodiments, be described by its ammonium nitrate, calcium chloride, calcium nitrate, or potassium sulfate concentration. The concentration of ammonium nitrate may be, for example, in a range from about 100 mg/L to about 1000 mg/L, about 100 mg/L to about 750 mg/L, about 100 mg/L to about 500 mg/L, about 100 mg/L to about 250 mg/L, or about 250 mg/L to about 500 mg/L, including all ranges derivable therebetween. The concentration of calcium chloride may be, for example, less than or equal to about 100 mg/L, greater than or equal to about 50 mg/L, about 50 mg/L to about 100 mg/L, or about 50 mg/L to about 75 mg/L, including all ranges derivable therebetween. The concentration of calcium nitrate may be, for example, less than or equal to about 500 mg/L, about 100 mg/L to about 500 mg/L, about 100 mg/L to about 300 mg/L, about 300 mg/L to about 400 mg/L, or about 100 mg/L to about 200 mg/L, including all ranges derivable therebetween. The concentration of potassium sulfate may be, for example, greater than about 500 mg/L, about 500 mg/L to about 750 mg/L, about 500 mg/L to about 1000 mg/L, about 500 mg/L to about 1500 mg/L, about 500 mg/L to about 2000 mg/L, about 750 mg/L to about 1000 mg/L, or about 1000 mg/L, including all ranges derivable therebetween.

In one aspect, monocot seed embryo explant is regenerated in contact with the regeneration medium at about 20° C. to about 32° C., 25° C. to about 29° C., or about 27° C. to about 28° C., including all ranges derivable therebetween. The monocot seed embryo explant may be regenerated in some embodiments for about 20 days to about 50 days or about 28 days to about 42 days, including all ranges derivable therebetween.

In another aspect, regenerating a genetically modified monocot plant or plant part on regeneration medium comprising a low salt concentration may improve transformation by at least about 0.1-fold, 0.2-fold, 0.3-fold, 0.4-fold, 0.5-fold, 0.6-fold, 0.7-fold, 0.8 fold, 0.9-fold, 1-fold, 1.5-fold, 2-fold, 2.5-fold, 3-fold, 3.5-fold, 4-fold, 4.5-fold, or 5-fold compared to genetically modified monocot plants or plant parts regenerated on a regeneration medium comprising a higher salt concentration. In addition, regenerating a genetically modified monocot plant or plant part on medium comprising a low salt concentration may increase rooting frequency by at least about 0.1-fold, 0.2-fold, 0.3-fold, 0.4-fold, 0.5-fold, 0.6-fold, 0.7-fold, 0.8 fold, 0.9-fold, 1-fold, 2-fold, 2.5-fold, 3-fold, 3.5-fold, 4-fold, 4.5-fold, or 5-fold compared to genetically modified monocot plants or plant parts regenerated on regeneration medium comprising higher salt concentration.

The regeneration step may also be carried out under a variety of lighting conditions. Some degree of lighting may generally be used during the second (or extended) bud induction step. According to some embodiments, the regeneration step may be carried out with an average or set light intensity of Photosynthetic Active Radiation (PAR) in a range from about 30 μ/m²·s to about 250 μ/m²·s, about 30 μ/m²·s to about 225 μ/m²·s, about 30 μ/m²·s to about 200 μ/m²·s, about 40 μ/m²·s to about 200 μ/m²·s, about 50 μ/m²·s to about 200 μ/m²·s, 50 μ/m²·s to about 180 μ/m²·s, 60 μ/m²·s to about 180 μ/m²·s, 70 μ/m²·s to about 180 μ/m²·s, 80 μ/m²·s to about 180 μ/m²·s, 90 μ/m²·s to about 180 μ/m²·s, 100 μ/m²·s to about 170 μ/m²·s, 110 μ/m²·s to about 160 μ/m²·s, or about 120 μ/m²·s to about 150 μ/m²·s, According to some embodiments, the second (or extended) bud induction step may be carried out with an average or set light intensity of Photosynthetic Active Radiation (PAR) at about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130 μ/m²·s, about 140 μ/m²·s, about 150 μ/m²·s, about 160 μ/m²·s, about 170 μ/m²·s, about 180 μ/m²·s, about 190 μ/m²·s, about 200 μ/m²·s, about 210 μ/m²·s, about 220 μ/m²·s, about 230 μ/m²·s, about 240 μ/m²·s, or about 250 μ/m²·s, According to some embodiments, different amounts of light and dark cycles may be used during the second (or extended) bud induction step, which may comprise a presence of lighting for a length of time between about 2 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the regeneration step may be about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

In still yet another aspect of the present invention, the monocot seed embryo explant and the genetically modified monocot plant or plant part are cultured and regenerated without producing a callus tissue culture.

In one aspect of the present invention, the regenerated genetically modified monocot plant or plant part is non-chimeric or has reduced chimerism. As used herein the term “chimeric” or “chimerism” refer to a plant, plant tissue, explant, or the like, which is composed of two genetically different types of tissues or cells with respect to a genetic modification.

### G. Genetically Modified Plants by Genetic Engineering

Various genetic engineering technologies have been developed and may be used by those of skill in the art to introduce transgenic or edited traits into plants. The methods generally involve the delivery of a polynucleotide sequence into a plant cell, which may typically be a heterologous and/or recombinant DNA molecule, which may comprise at least one transgene or expression cassette or an RNA molecule, such as a guide RNA (gRNA) or part of a ribonucleoprotein (RNP), such as a gRNA/site-directed nuclease complex for genome editing. In certain aspects of the present embodiments, traits are introduced into monocot plants via altering or introducing a single genetic locus or transgene into the genome of a plant. Methods of genetic engineering to modify, delete, or insert transgenes, edits, mutations and polynucleotide sequences into the genomic DNA of plants are known in the art. Molecular methods of editing a plant cell genome or endogenous plant gene using a genome editing technique is known in the art. According to present embodiments, a polynucleotide or DNA molecule comprising and/or encoding genome editing tools or machinery, such as a guide RNA, site-directed nuclease and/or template DNA molecule, may be introduced into a plant cell using the methods described herein.

In some embodiments, transformed monocot plants can be created through the site-specific modification of a plant genome. Methods of site directed integration of a transgene or polynucleotide sequence include, for example, utilizing sequence-specific nucleases, such as zinc-finger nucleases (see, for example, U.S. Pat. Appl. Pub. No. 2011/0203012); engineered or native meganucleases; TALE-endonucleases (see, for example, U.S. Pat. Nos. 8,586,363 and 9,181,535); RNA-guided endonucleases, such as those of the CRISPR/Cas systems (see, for example, U.S. Pat. Nos. 8,697,359, 8,771,945 and 9,790,490 and U.S. Pat. Appl. Pub. No. 2014/0068797) and CRISPR-associated transposases or CAST (see, for example US Patent Application Pub. No. 2020/0190487), the entire contents and disclosures of which are incorporated herein by reference. Some embodiments thus relate to utilizing a nuclease or any associated protein to carry out genome modification. This nuclease could be provided heterologously within a donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a plant genome (see, for example Sauer et al., Plant Physiol, 170 (4): 1917-1928, 2016, the entire content and disclosure of which is incorporated herein by reference). Other methods for altering a genetic locus include, for example, utilizing CRISPR/Cas Base-editors or Prime-editors to introduce single or multiple base pair modifications in a plant genome (see, for example, Komor et al., Nature 533, 420-424 (2016); Gaudelli et. al., Nature 551, 464-471 (2017); Komor, et. al., Science Advances 3: (8) (2017), and Rees, et. al., Nat Rev Genet. 2018 Dec. 19(12): 770-788, PCT Patent Application Pub. No. WO 2020/191248, the entire contents and disclosures of which are incorporated herein by reference).

Methods for site-directed alteration or introduction/integration of a heterologous and/or recombinant genetic sequence or transgene are known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick by non-homologous end joining (NHEJ) or by homologous recombination (HR) between the homology arm(s) of the desired sequence and the target sequence. This could result in site-directed integration of all or part of the donor template, transgene or expression cassette polynucleotide into the target site for the nuclease in the genome to create the targeted insertion event. The presence of homology arms in the DNA to be integrated which may promote the adoption and targeting of the insertion sequence or part of the insertion sequence into the plant genome during the repair process through homologous recombination or non-homologous end joining (NHEJ).

In other embodiments, genetic modification of a plant may comprise transformation of a plant, plant part, plant tissue or plant cell to insert a polynucleotide or DNA sequence or transgene into the genome of the plant, plant part, plant tissue or plant cell. Methods for transformation of plants that are known in the art and applicable to many crop species include, but are not limited to, electroporation, microprojectile or particle bombardment, microinjection, PEG-mediated transformation, Agrobacterium-mediated transformation, and other modes of direct DNA uptake. Bacteria known to mediate plant cell transformation include a number of species of bacterial genera, species, and strains that may be assigned to the order Rhizobiales (or Hyphomicrobiales) other than Agrobacterium, including but not limited to, bacterial species and strains from the taxonomic families Rhizobiaceae (e.g. Rhizobium spp., Sinorhizobium spp.), Phyllobacteriaceae (e.g. Mesorhizobium spp., Phyllobacterium spp.), Brucellaceae (e.g. Ochrobactrum spp.), Bradyrhizobiaceae (e.g. Bradyrhizobium spp.), and Xanthobacteraceae (e.g. Azorhizobium spp.), among others. According to some embodiments, Agrobacterium-mediated transformation is mediated by Agrobacterium tumefaciens. Targets for such transformation have often been undifferentiated callus tissues, although differentiated tissue also has been used for transient and stable plant transformation. As is well known in the art, other methods for plant transformation may be utilized, for instance as described by Miki et al., (1993, “Procedures for Introducing Foreign DNA into Plants,” in Methods in Plant Molecular Biology and Biotechnology, Glick B. R. and Thompson, J. E. Eds., CRC Press, Inc., Boca Raton, pages 67-88).

In specific embodiments, microprojectile bombardment may be employed to deliver transforming a polynucleotide or DNA molecule, vector, sequence or segment to at least one cell of a plant explant(s). In this method, particles are coated with a polynucleotide or polynucleotide/protein complex and delivered into cells by a propelling force. Exemplary particles may include those comprised of tungsten, platinum, or gold. For bombardment, explants or other target cells may be arranged on solid culture medium. The cells to be bombarded are positioned at an appropriate distance below the projectile stopping plate. A polynucleotide may be delivered into plant cells by acceleration using a biolistics particle delivery system, which may propel particles coated with a DNA or polynucleotide molecule through a screen, such as a stainless steel or Nytex screen, and toward the explants positioned on a surface. The screen may disperse the particles so that they are not delivered to the recipient cells in large aggregates. Microprojectile bombardment techniques are widely applicable and may be used to transform a variety of plant species.

Agrobacterium-mediated or Rhizobiales-mediated transformation of explants is another widely applicable system for introducing heterologous and/or recombinant DNA molecules into plant cells. Modern Agrobacterium transformation vectors are capable of replication in E. coli as well as Agrobacterium, allowing for convenient manipulations (see, e.g., Klec et al., Nat. Biotechnol., 3 (7): 637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti plasmids can be used for transformation. Agrobacterium-mediated transformation is often the method of choice for many plant species. The use of Agrobacterium-mediated plant integrating vectors to introduce DNA into plant cells is known in the art (see, e.g., Fraley et al., Nat. Biotechnol., 3:629-635, 1985; U.S. Pat. No. 5,563,055).

A number of promoters and expression elements have utility for plant gene expression for any selectable marker, scoreable marker, transgene, or any other gene of agronomic interest. Promoters may include any constitutive promoter, tissue specific promoters, organ specific promoters, inducible promoters, reproductive tissue promoter, developmental stage promoter, viral promoter, etc. Examples of various types of promoters and expression elements are known in art. Expression elements that may be useful for plant gene expression may include, for example, various promoters, enhancers, leaders, 5′ and 3′ untranslated regions, introns, terminators, etc., as known in the art. A selectable or screenable marker or gene of interest may also be fused to a transmit peptide or other targeting sequence. Transport of proteins produced by transgenes to a subcellular compartment such as the chloroplast, vacuole, peroxisome, glyoxysome, cell wall, nucleus, or mitochondrion or for secretion into the apoplast, may be accomplished by means of operably linking the nucleotide sequence encoding a signal or targeting sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized. The presence of a signal sequence directs a polypeptide to either an intracellular organelle or subcellular compartment or for secretion to the apoplast. Many signal sequences are known in the art. See, for example Becker et al. (Plant Mol. Biol., 20:49, 1992); Knox et al. (Plant Mol. Biol., 9:3-17, 1987); Lerner et al. (Plant Physiol., 91:124-129, 1989); Fontes et al. (Plant Cell, 3:483-496, 1991); Matsuoka et al. (Proc. Natl. Acad. Sci. USA, 88:834, 1991); Gould et al. (J. Cell. Biol., 108:1657, 1989); Creissen et al. (Plant J., 2:129, 1991); Kalderon et al. (Cell, 39:499-509, 1984); Steifel et al. (Plant Cell, 2:785-793, 1990).

Examples of constitutive promoters may include, for example, the cauliflower mosaic virus (CaMV) 35S promoter, which confers constitutive, high-level expression in most plant tissues (see, e.g., Odel et al., Nature, 313:810, 1985), including monocots (see, e.g., Dekeyser et al., Plant Cell, 2:591, 1990; Terada and Shimamoto, Mol. Gen. Genet., 220:389, 1990); a tandemly duplicated version of the CaMV 35S promoter, the enhanced 35S promoter (e35S), the nopaline synthase promoter (An et al., Plant Physiol., 88:547, 1988), the octopine synthase promoter (Fromm et al., Plant Cell, 1:977, 1989); and the figwort mosaic virus (FMV) promoter as described in U.S. Pat. No. 5,378,619 and an enhanced version of the FMV promoter (cFMV) where the promoter sequence of FMV is duplicated in tandem, the cauliflower mosaic virus 19S promoter, a sugarcane bacilliform virus promoter, a commelina yellow mottle virus promoter, and other plant DNA virus promoters known to express in plant cells.

With an inducible promoter, the rate of transcription increases in response to an inducing agent. Any inducible promoter can be used in the instant invention. A variety of plant gene promoters that are regulated in response to environmental, hormonal, chemical, and/or developmental signals can be used for expression of an operably linked gene in plant cells, including promoters regulated by (1) heat (Callis et al., Plant Physiol., 88:965, 1988), (2) light (e.g., pea rbcS-3A promoter, Kuhlemeier et al., Plant Cell, 1:471, 1989; maize rbcS promoter, Schaffner and Sheen, Plant Cell, 3:997, 1991; or chlorophyll a/b-binding protein promoter, Simpson et al., EMBO J., 4:2723, 1985), (3) hormones, such as abscisic acid (Marcotte et al., Plant Cell, 1:969, 1989), (4) wounding (e.g., wunl, Siebertz et al., Plant Cell, 1:961, 1989); or (5) chemicals such as methyl jasmonate, salicylic acid, or Safener. It may also be advantageous to employ organ-specific or tissue specific promoters known in the art (e.g., Roshal et al., EMBO J., 6:1155, 1987; Schernthaner et al., EMBO J., 7:1249, 1988; Bustos et al., Plant Cell, 1:839, 1989).

Exemplary polynucleotide or DNA molecules which may be introduced to the monocot plants include, for example, DNA sequences or genes from another species, or even genes or sequences which originate with or are present in the same species, but are incorporated into recipient cells by genetic engineering methods rather than classical reproduction or breeding techniques. However, the term “exogenous” is also intended to refer to genes that are not normally present in the cell being transformed, or perhaps not present in the form, structure, location, etc. A polynucleotide may include a DNA molecule or sequence which is already present in the plant cell, is from another plant, is from a different organism, is exogenous or generated externally. A transgene or expression cassette may encode a mRNA and protein or an RNA molecule for suppression, such as a miRNA, siRNA, dsRNA, antisense RNA, inverted repeat RNA, etc. A polynucleotide may be an exogenous, heterologous and/or recombinant polynucleotide or DNA molecule or sequence.

Many hundreds if not thousands of different genes are known and could potentially be introduced into a plant according to the invention. Non-limiting examples of particular genes and corresponding phenotypes one may choose to introduce into a monocot plant include one or more genes for insect tolerance, such as a Bacillus thuringiensis (B.t.) gene, pest tolerance such as genes for fungal disease control, herbicide tolerance such as genes conferring glyphosate tolerance, and genes for quality improvements such as yield, nutritional enhancements, environmental or stress tolerances, or any desirable changes in plant physiology, growth, development, morphology or plant product(s). For example, structural genes would include any gene that confers insect tolerance including but not limited to a Bacillus insect control protein gene as described in WO 99/31248, herein incorporated by reference in its entirety, U.S. Pat. No. 5,689,052, herein incorporated by reference in its entirety, U.S. Pat. Nos. 5,500,365 and 5,880,275, herein incorporated by reference in their entirety. In another embodiment, the structural gene can confer tolerance to the herbicide glyphosate as conferred by genes including, but not limited to Agrobacterium strain CP4 glyphosate resistant EPSPS gene (aroA:CP4) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety.

A variety of assays are known in the art and may be used to confirm the presence of an exogenous DNA sequence or transgene in transformed, edited or genetically modified plants. Such assays include, but are not limited to, Southern blotting. Northern blotting, sequencing, PCR, in situ hybridization, ELISA, Western blotting, enzymatic function assays, plant part assays, or by analyzing the phenotype of a regenerated plant.

### H. Culture Media

A variety of tissue culture media are known that, when supplemented appropriately, support plant tissue growth and development, including formation of mature plants from excised plant tissue. As used herein the term “tissue culture media” refers to liquid, semi-solid, or solid media used to support plant growth and development in a non-soil environment. These tissue culture media can either be purchased as a commercial preparation or custom prepared and modified by those of skill in the art. Examples of such media include, but are not limited to, those described by Murashige and Skoog. (1962); Chu et al., (1975); Linsmaier and Skoog, (1965); Uchimiya and Murashige, (1962); Gamborg et al., (1968); Duncan et al., (1985); Mccown and Lloyd, (1981); Nitsch and Nitsch (1969); and Schenk and Hildebrandt, (1972), or derivations of these media supplemented accordingly. Those of skill in the art are aware that media and media supplements, such as nutrients and plant growth regulators for use in transformation and regeneration are usually optimized for the particular target crop or variety of interest. Tissue culture media may be supplemented with carbohydrates such as, but not limited to, glucose, sucrose, maltose, mannose, fructose, lactose, galactose, and/or dextrose, or ratios of carbohydrates. Reagents are commercially available and can be purchased from a number of suppliers (see, for example Sigma Chemical Co., St. Louis, MO; and PhytoTechnology Laboratories, Shawnee Mission, KS). These tissue culture media may be used to prepare an inoculation, co-culture, bud induction, second induction, or regeneration media and in particular embodiments may comprise a selection agent.

### I. Selectable Markers

In particular embodiments, media for use according to the present invention may comprise one or more selection agents and the heterologous polynucleotide molecule for use in the present invention may comprise a selectable marker gene, wherein the selectable marker gene provides resistance to the selection agent. As used herein, “selectable marker” or “screenable marker” or “scoreable marker” refers to a nucleic acid sequence whose expression confers a phenotype facilitating identification of cells containing the nucleic acid sequence. Examples of various selectable markers and genes providing resistance to them are disclosed in Miki and McHugh, 2004. Selectable marker genes that may be used include, but are in no way limited to, aroA, EPSPS, aadA, pat, bar, hph (hygromycin B phosphotransferase), DMO (dicamba monooxygenase) and NPT II. Non-limiting examples of selection agents that may be used according to the present invention include glyphosate, glufosinate, phosphinothricin, bromoxynil, bialaphos, dicamba, imidazolinone, sulfonylurea, acetolactate synthase inhibitors, protoporphyrinogen oxidase inhibitors, hydroxyphenyl-pyruvate-dioxygenase inhibitors, antibiotic inhibitors, neomycin, kanamycin, paramomycin, G418, aminoglycosides, spectinomycin, streptomycin, hygromycin B, bleomycin, phleomycin, sulfonamides, gentamycin, streptothricin, chloramphenicol, methotrexate, 2-deoxyglucose, betaine aldehyde, S-aminoethyl L-cysteine, 4-methyltryptophan, D-xylose, D-mannose, and benzyladenine-N-3-glucuronidase. The heterologous polynucleotide molecule for use in the present invention may, in some embodiments, comprise two or more selectable marker genes. Selection agents for use in the present invention may, in some embodiments, be used alone or as a combination of two or more selection agents. In one embodiment, the embodiments of the present inventions may be performed in the absence of any selection agent.

According to embodiments of the present invention, the insertion sequence of an exogenous polynucleotide or DNA molecule for transformation or genome editing may comprise a plant selectable marker gene to allow for successful selection for, and production of, transformed or transgenic R₀ plants. A plant selectable marker gene or transgene may include any gene conferring tolerance to a corresponding selection agent, such that plant cells transformed with the plant selectable marker transgene may tolerate and withstand the selection pressure imposed by the selection agent. As a result, transformed plant cells of an explant are favored to grow, proliferate, develop, etc., under selection. Although a plant selectable marker gene is generally used to confer tolerance to a selection agent, additional screenable marker gene(s) may also be used in addition to the selectable marker, perhaps also along with a gene of agronomic interest. Such screenable marker genes may include, for example, uidA for β-glucuronidase (GUS; e.g., as described in U.S. Pat. No. 5,599,670, which is hereby incorporated by reference) or gfp for green fluorescent protein and variants thereof (GFP described in U.S. Pat. Nos. 5,491,084 and 6,146,826, both of which are hereby incorporated by reference) or crtB for phytoene synthase (e.g., as described in U.S. Pat. Nos. 8,237,016 and 10,240,165, both of which are hereby incorporated by reference. Additional examples of screenable markers may include secretable markers whose expression causes secretion of a molecule(s) that can be detected as a means for identifying transformed cells.

A plant selectable marker gene may comprise a gene encoding a protein that provides or confers tolerance or resistance to an herbicide, such as glyphosate and glufosinate. Useful plant selectable marker genes known in the art may include those encoding proteins that confer resistance or tolerance to streptomycin or spectinomycin (e.g., aadA, spec/strep), kanamycin (e.g., nptII), hygromycin B (e.g., aph IV), gentamycin (e.g., aac3 and aacC4), and chloramphenicol (e.g., CAT). Additional examples of known plant selectable marker genes encoding proteins that confer herbicide resistance or tolerance include, for example, a transcribable DNA molecule encoding 5-enolpyruvylshikimate-3-phosphate synthase (EPSPS for glyphosate tolerance; e.g., as described in U.S. Pat. Nos. 5,627,061; 5,633,435; 6,040,497; and 5,094,945, all of which are hereby incorporated by reference); a transcribable DNA molecule encoding a glyphosate oxidoreductase and a glyphosate-N-acetyl transferase (GOX; e.g., as described in U.S. Pat. No. 5,463,175); GAT described in U.S. Patent publication No. 20030083480; a transcribable DNA molecule encoding phytoene desaturase (crtI; e.g., as described in Misawa, et al., Plant Journal, 4:833-840 (1993) and Misawa, et al., Plant Journal, 6:481-489 (1994) for norflurazon tolerance, incorporated herein by reference); and the bar gene (e.g., as described in DeBlock, et al., EMBO Journal, 6:2513-2519 (1987) for glufosinate and bialaphos tolerance, incorporated herein by reference).

The insertion sequence of an exogenous DNA molecule may further comprise sequences for removal of one or more transgene(s) or expression cassette(s), such as a plant selectable marker transgene, or any portion or sequence thereof, after successful production and/or confirmation of a transformed plant(s), especially after the transgene or expression cassette is no longer needed. In some embodiments, this may be accomplished by flanking the transgene sequence to be removed, with known or later developed recombination sites (e.g., LoxP sites, FRT sites, etc.) that can be recognized and removed by an endogenous or exogenously provided recombinase enzyme (e.g., Cre, Flp, etc.). The recombinase enzyme may be introduced and expressed in trans, such as by crossing the transformed plant to another plant having the recombinase transgene, to accomplish excision of the transgene. Accordingly, the unwanted sequence element or transgene can be removed once its use or purpose has expired, thus preventing its further expression or transmission in the germ line.

### EXAMPLES

Those of skill in the art will appreciate the many advantages of the methods and compositions provided by the present invention. The following examples are included to demonstrate the preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention. All references cited herein are incorporated herein by reference to the extent that they supplement, explain, provide a background for, or teach methodology, techniques, or compositions employed herein.

### Example 1

### Transformation of Corn Excised Explants

This example describes a method of Agrobacterium-mediated transformation of embryo explants excised from dry mature corn seeds, including the steps of explant preparation, Agrobacterium inoculation and co-culture, bud induction, extended bud induction, and regeneration of transgenic plants.

### Explant Preparation

Explants were excised from dry mature 01DKD2 corn seeds and stored in sealed pouches at −20° C. The explants were removed from the freezer and allowed to equilibrate to room temperature for at least 30 minutes prior to explant preparation. Explants were surface-sterilized with 70% ethanol containing 100 g/L polyethylene glycol (PEG) molecular weight (MW) 800 in an appropriately sized roller bottle, which was agitated by rolling slowly for about 3.5 minutes. Approximately 300 mL of sterilization solution was used for up to about 15,000 corn explants and about 500 mL of sterilization solution was used for about 15,000 to about 30,000 corn explants. Following sterilization, the corn explants and sterilization solution were poured over a large steel strainer. The explants retained on the strainer were rinsed with about 2.5 to about 3.0 L of sterile water to remove any significant remaining sterilization solution and then transferred to a sterile glass beaker.

Following sterilization, the corn explants were floatation enriched to remove debris. Briefly, 500 mL of sterile water was poured into the sterile glass beaker comprising the explants and explants that floated to the surface were collected by pouring into a large strainer. Floatation was repeated several times until no remaining viable explants floated to the surface.

Corn explants collected following the floatation enrichment were transferred into a new sterile glass beaker for rehydration. Briefly, 400 mL of rehydration medium comprising ⅖ strength of B5 macro salts except for CaCl₂), which is ½ strength, 1/10 strength of B5 micro salts and vitamins, 1 g/L potassium nitrate, 30 g/L dextrose, 2.8 mg/L sequestrene, 3.9 g/L 2-(N-morpholino) ethanesulfonic acid (MES), and 0.03 g/L Clearys 3336 WP, pH 5.4 was added to the beaker. The beaker was then covered with aluminum foil and incubated for about 1 to about 2 hours. The components for each of the MS, Gamborg B5 (B5), and Woody Plant Medium (WPM) salts and vitamins are known in the art and are provided together in Table 1. See, for example, Murashige and Skoog (1962) Plant Physiology, 15:473-497; O L Gamborg, et al. (1968) Exp Cell Res., 50 (1): 151-8; and Mccown and Lloyd (1981) HortScience, 16:453-453, the entire content and disclosure of each of which is incorporated herein by reference.

TABLE 1
Components of MS, B5, WPM and Low Nitrogen MS salts and vitamins.

| | MS | | WPM | | B5 | | Low Nitrogen MS | |
|---|---|---|---|---|---|---|---|---|
| Components | mg/L | mM | mg/L | mM | mg/L | mM | mg/L | mM |
| Ammonium | 1650 | 20.6 | 400 | 5.0 | — | — | — | — |
| nitrate | | | | | | | | |
| Ammonium | — | — | — | — | 134 | 1.01 | 463 | 3.5 |
| sulfate | | | | | | | | |
| Boric acid | 6.2 | 0.1 | 6.2 | 0.1 | 3.0 | 0.049 | 6.2 | 0.1 |
| Calcium chloride, | 332.2 | 3.0 | 72.5 | 0.65 | 113.24 | 1.02 | — | — |
| anhydrous | | | | | | | | |
| Calcium chloride, | — | — | — | — | — | — | 300 | 2.04 |
| dihydrate | | | | | | | | |
| Calcium nitrate | — | — | 386 | 3.78 | — | — | — | — |
| Cobalt | 0.03 | 0.0001 | — | — | 0.025 | 0.0001 | — | 0.0001 |
| chloride•6H₂O | | | | | | | | |
| Cupric | 0.03 | 0.0001 | 0.25 | 0.001 | 0.025 | 0.0001 | 0.025 | 0.0001 |
| sulfate•5H₂O | | | | | | | | |
| Na₂ EDTA•2H₂O | 37.3 | 0.1 | 37.3 | 0.1 | 37.3 | 0.1 | 37.3 | 0.1 |
| Ferrous | 27.8 | 0.1 | 27.85 | 0.1 | 27.8 | 0.1 | 27.8 | 0.1 |
| sulfate•7H₂O | | | | | | | | |
| Magnesium | 180.7 | 1.5 | 180.7 | 1.5 | 122.09 | 1.01 | — | — |
| sulfate, | | | | | | | | |
| anhydrous | | | | | | | | |
| Magnesium | — | — | — | — | — | — | 370 | 1.5 |
| sulfate, | | | | | | | | |
| heptahydrate | | | | | | | | |
| Manganese | 16.9 | 0.11 | 22.3 | 0.15 | 10 | 0.066 | 16.9 | 0.11 |
| sulfate•H₂O | | | | | | | | |
| Molybdic acid (Na | 0.25 | 0.001 | 0.25 | 0.001 | 0.25 | 0.001 | 0.25 | 0.001 |
| salt)•2H₂O | | | | | | | | |
| Potassium iodide | 0.83 | 0.005 | — | — | 0.75 | 0.0045 | 0.83 | 0.005 |
| Potassium nitrate | 1900 | 18.8 | — | — | 2500 | 24.73 | 2830 | 27.99 |
| Potassium | 170 | 1.25 | 170 | 1.25 | — | — | 185 | 1.36 |
| phosphate, | | | | | | | | |
| monobasic | | | | | | | | |
| Potassium sulfate | — | — | 990 | 5.68 | — | — | — | — |
| Sodium | — | — | — | — | 150 | 1.25 | — | — |
| phosphate | | | | | | | | |
| monobasic | | | | | | | | |
| Zinc sulfate•7H₂O | 8.6 | 0.03 | 8.6 | 0.03 | 2 | 0.007 | 8.6 | 0.03 |
| | | | | | | | — | |
| Myo-inositol | 100 | | 100 | | 100 | | 100 | |
| Nicotinic acid | 0.5 | | 0.5 | | 1 | | 1 | |
| (free acid) | | | | | | | | |
| Pyridoxine•HCl | 0.5 | | 0.5 | | 1 | | 1 | |
| Thiamine•HCl | 0.1 | | 1 | | 10 | | 10 | |
| Glycine (free base) | 2 | | 2 | | — | | — | |
| Total Salt (mg/L) | 4330.83 | | 2301.95 | | 3100.48 | | 4245.93 | |

In Table 1, the major salts or macronutrients are in bold, the minor salts or micronutrients are in normal font, and the vitamins are in italics. Each of these salt and vitamin mixtures are available commercially, such as from PhytoTechnology Laboratories (for example, MS salt mixture catalog number is M519, B5 salt mixture catalog number is G398, WPM salt mixture catalog number is L449, and Low Nitrogen MS mixture catalog numbers are a combination of M529 and G249). The basal salt mixture includes both macronutrients and micronutrients. The concentrations are provided in solution or when a specified amount is added per 1 L total volume water, which may be adjusted for any other volume. Dilutions are made relative to these full concentrations and may be specified as a fraction. The full concentration of salts or vitamins in Table 1 is included in a solution described herein unless a dilution is specified.

### Agrobacterium Preparation

Agrobacterium glycerol stock AB32 was retrieved from a −80° C. freezer and thawed at ambient temperature in a laminar flow hood. After thorough mixing by vortexing, 250 mL of the thawed Agrobacterium glycerol stock was inoculated into 250 mL of liquid LB medium containing appropriate antibiotics for the transformation constructs in a sterile 1 L flask. Appropriate antibiotics may include, for example 50 mg/L spectinomycin or 30 mg/L gentamicin. The flask was placed into an orbital shaker/incubator set to 200 rpm and cultured at 27.5±2° C. in the dark for 16-24 hours, or until the optical density measurement at 660 nm (OD₆₆₀) of the inoculum was within a range of 0.6-1.2. Following centrifugation of the overnight Agrobacterium culture at approximately 3000 rpm or 2620×g (Sorvall® 3B, 6000A rotor) at 4° C. for 25 minutes, the pellet was resuspended in 50 mL of inoculation medium comprising ⅖ strength of B5 macro salts, 1/10 of B5 micro salts and vitamins, 1 g/L potassium nitrate, 30 g/L dextrose, and 3.9 g/L MES. Following resuspension, Agrobacterium from all tubes were pooled and mixed well and a 1:20 dilution of the Agrobacterium suspension was made for OD₆₆₀ measurement. The concentrated Agrobacterium suspension was then diluted to a final OD₆₆₀ of 0.25 in the inoculation medium.

### Inoculation and Co-Culture

Following rehydration as described above, explants were collected by pouring the explant/rehydration mixture into a sterile stainless steel mesh strainer. The retained explants were quickly blotted onto a sterile towel to remove excess liquid. Approximately 2,500 corn explants and 40 ml of Agrobacterium suspension were added to a 50 mL vented conical tube. The Agrobacterium suspension covered all explants in the tube. The tube was then placed in a vacuum chamber where about 300 psi of pressure was applied for about 3 minutes. After the pressure was slowly released, the tubes were centrifuged at 2,620×g at 4° C. for 30 minutes. Following inoculation with pressure and centrifugation, the Agrobacterium suspension was removed by transferring the explants into a sterile stainless-steel strainer. Excess liquid was removed by tapping the strainer onto sterile paper towels.

The inoculated corn explants were transferred to co-culture plates (25 mm×100 mm) containing a piece of sterile Whatman #1 filter paper (82 mm) wetted with 1.25 mL of rehydration medium. Each plate contained approximately 500-600 corn explants evenly spread out across the plate in a single layer. The co-culture plates were incubated at 20° C. and about 65% relative humidity with a photoperiod of 16 hours light/8 hours dark for κ-6 days. The targeted light intensity was about 90 μ/m²·s of Photosynthetic Active Radiation (PAR).

### Bud Induction

Following the co-culture step, corn explants were transferred to a solid bud induction medium, for instance comprising MS salts, B5 vitamins, 30 g/L sucrose, 0.69 g/L proline, 1 g/L NZ amine-A (casein enzymatic hydrolysate; Millipore Sigma), 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, and 3.5 g/L low EEO agarose, pH 5.8. Explants from each co-culture plate were evenly plated onto five plates containing the bud induction medium. The plates were incubated in a Percival® chamber at 33° C. (35° C. actual plate temperature) with a photoperiod of 16 hours light/8 hours dark for 6-8 days at a light intensity of about 90 μ/m²·s of PAR.

### Extended Bud Induction

Following the bud induction step, corn explants were transferred to a solid extended bud induction medium comprising MS salts, B5 vitamins, 60 g/L sucrose, 0.5 g/L glutamine, 0.69 g/L proline, 1 g/L NZ amine-A, 2 mg/L glycine, 1.95 g/L MES, 1.25 mg/L cupric sulfate, 2 mg/L thidiazuron, 2 mg/L picloram, 400 mg/L carbenicillin, 200 mg/L cefotaxime, 100 mg/L timentin, 25 μM glyphosate and 3.5 g/L low EEO agarose, pH 5.8. Explants from each bud induction plate were divided evenly onto two plates containing the extended bud induction medium. These plates were cultured at 28° C., with a photoperiod of 16 hours light/8 hours dark for about 5-18 days at a light intensity of about 150 μ/m²·s of PAR.

### Regeneration

Explants from the extended bud induction plates were transferred to a solid regeneration medium comprising LM Woody Plant Medium (WPM) salts and vitamins, 0.03 g/L Cleary 3336 WP (thiophanate-methyl), 30 g/L sucrose, 0.69 g/L proline, 2 mg/L glycine, 1 g/L MES, 3.5 g/L low EEO agarose, 400 mg/L carbenicillin, 200 mg/L cefotaxime, 100 mg/L timentin and 20 μM glyphosate, pH 5.8 in 9 cm Vivi® trays (Vivi®), The Netherlands) for shoot development and rooting. After the explants were evenly distributed on the regeneration medium, the Vivi® trays were sealed with plastic film using a hand-held sealing iron and cultured at 28° C. and ambient humidity with a photoperiod of 16 hours light/8 hours dark for 28-42 days at a light intensity of about 160 μ/m²·s of PAR. Following the regeneration step, typically about 10-11 weeks after the inoculation step, putative transgenic plants with visible roots and no sign of chimerism were transplanted in soil plugs (2″ diameter×3″ height (Gro-Tech, Rough and Ready, CA 95975) for further growth and development.

### Example 2

### Transformation of Wheat Excised Explants

This example describes a method of Agrobacterium-mediated transformation of embryo explants excised from dry mature wheat seeds, including the steps of explant preparation, Agrobacterium inoculation and co-culture, bud induction, extended bud induction, and regeneration of transgenic plants.

### Explant Preparation

Explants were excised from dry mature wheat seeds and were stored in sealed pouches at −20° C. The explants were removed from the freezer and allowed to equilibrate to room temperature for at least 30 minutes prior to explant preparation. About 10,000 explants were surface-sterilized by placing the explants into a sterile 1 L bottle with about 500 mL of 70% ethanol. The bottle was agitated by rolling or shaking gently by hand for about 5 minutes. Following surface sterilization and removal of the 70% ethanol solution, wheat explants were rinsed with sterile water to remove any significant remaining sterilization medium.

The wheat explants were floatation enriched to remove debris. Briefly, explants in sterile water were poured into a sterile sieve/collection vessel apparatus including an outside container and an inner sieve container. The inner sieve container fits inside the outside container and has a 26×26 stainless steel mesh melted onto the bottom. After the explants were transferred into the apparatus, sterile water was added. The inner sieve container was raised out of and lowered into the water repeatedly to induce floatation of the wheat explants. Floating explants were collected using a vacuum collector. This floatation step was repeated several times until about 50% of the floating material was debris. Following the floatation step, debris was discarded. The collected wheat explants were then added to 50 mL centrifuge tubes using a sterile spatula for Agrobacterium inoculation. About 5,000 wheat explants were added per tube.

### Agrobacterium Preparation

Agrobacterium glycerol stock was retrieved from a −80° C. freezer and thawed at ambient temperature in a laminar flow hood. After thorough mixing by vortexing, 500 mL of the thawed Agrobacterium glycerol stock was inoculated into 250 mL of liquid LB medium containing appropriate antibiotics for the transformation constructs in a 1 L sterile flask on a shaker at 170 rpm and cultured at 27-28° C. in the dark for about 16-18 hour, or until the OD₆₆₀ was within a range of 0.6-1.6. Appropriate antibiotics may include, for example, 50 mg/L spectinomycin or 30 mg/L gentamicin. Following centrifugation of the overnight Agrobacterium culture at approximately 3500-3700 rpm at 20° C. for 20 minutes, the pellet was resuspended in 25 mL of inoculation medium comprising 1/10 of MS salts, MS vitamins, 0.5 g/L glutamine, 0.1 g/L casein hydrolysate, 0.75 g/L magnesium chloride, 1.95 g/L MES, 40 g/L maltose and 100 mg/L ascorbic acid, pH 5.8. Following resuspension, Agrobacterium from all centrifuge tubes were pooled and mixed well and a 1:20 dilution of the Agrobacterium suspension was made for an OD₆₆₀ measurement. The concentrated Agrobacterium suspension was then diluted to a final OD₆₆₀ of 0.5 in the inoculation medium. The prepared Agrobacterium suspension was stored at 4° C. for up to 8 hours until use in transformation.

### Inoculation and Co-Culture

About 40 to 50 mL of the Agrobacterium suspension prepared as described above was added to each 50 mL centrifuge tube containing the prepared wheat explants. The tubes containing the Agrobacterium and the explants were centrifuged at 1400×g at 4° C. for 30 min. Following centrifugation, the explants and Agrobacterium were resuspended by shaking, and the explants were allowed to settle down to the bottom of the tubes. The Agrobacterium suspension was removed by decanting. The inoculated wheat explants were then transferred to a 100 mm×25 mm Petri plate using a sterile spatula or loop and the remaining Agrobacterium suspension was removed using a sterile transfer pipette. Alternatively, a sterile stainless-steel sieve may be used to collect the explants and to remove the excess Agrobacterium suspension as described in Example 1.

The inoculated explants were then transferred to co-culture plates (25 mm×100 mm) containing a piece of sterile Ahlstrom filter paper wetted with 1.25 mL of inoculation medium. Each co-culture plate contained approximately 500-600 wheat explants evenly spread out across the plate in a single layer. The co-culture plates were incubated at 23° C. and about 70% relative humidity in the dark for about 70% for 3-4 days.

### Delay

After the co-culture step, the filter paper with explants were lifted from the co-culture plates using sterile forceps and directly transferred to plates containing solid delay medium comprising 0.78 g/L MS basal salts with no nitrogen, MS vitamins, 1.64 g/L potassium sulfate, 4.95 g/L ammonium nitrate, 60 g/L maltose, 0.5 g/L glutamine, 1 g/L NZ amine-A, 0.75 g/L magnesium chloride hexahydrate, 1.95 g/L MES, 1.25 mg/L cupric sulfate, 3 mg/L thidiazuron, 2 mg/L picloram, 200 mg/L carbenicillin, 100 mg/L cefotaxime and 3.5 g/L agarose low EEO, pH 5.8. The plates were then cultured at 25° C. and about 35% relative humidity with a photoperiod of 16 hours light/8 hours dark for 12-16 days.

### Selection

Following the delay step, selection was carried out using a liquid medium. Briefly, wheat explants on delay medium were transferred to selection plates containing 2 pieces of felt, 1 piece of filter paper with a hole punched through the paper to assist with aspiration, and 25 mL of selection medium comprising 0.78 g/L MS basal salts with no nitrogen, MS vitamins, 1.64 g/L potassium sulfate, 4.95 g/L ammonium nitrate, 30 g/L maltose, 0.5 g/L glutamine, 1 g/L NZ amine-A, 0.75 g/L magnesium chloride, 1.95 g/L MES, 1.25 mg/L cupric sulfate, 3 mg/L thidiazuron, 2 mg/L picloram, 200 mg/L carbenicillin, 100 mg/L cefotaxime and 30 μM glyphosate, pH 5.8. Approximately 80-100 wheat explants were placed into each selection plate. The plates were cultured at 25° C. and a relative humidity of about 35% with a photoperiod of 16 hours light/8 hours dark for 12-16 days.

### Regeneration

Following the selection step, the selection medium was aspirated and 20 mL of liquid regeneration medium comprising MS basal salts, MS vitamins, 30 g/L sucrose, 0.69 g/L proline, 1 g/L MES, 400 mg/L carbenicillin, 200 mg/L cefotaxime, 100 mg/L timentin and 30 μM glyphosate, pH 5.8 was added. The plates were cultured at 25° C. and about 35% relative humidity with a photoperiod of 16 hours light/8 hours dark for 12-16 days (Regeneration Step 1). The regeneration medium was then aspirated and replaced with 15 mL of fresh regeneration medium. The plates were then incubated at 25° C. and about 35% relative humidity with a photoperiod of 16 hours light/8 hours dark for an additional 5-9 days (Regeneration Step 2).

Following Regeneration Step 2, 10 mL of fresh regeneration medium was added to each plate. The plates were then cultured at 25° C. and about 35% relative humidity with a photoperiod of 16 hours light/8 hours dark until regenerated shoots were ready to be transplanted.

Typically at about 60-70 days post inoculation, regenerated shoots were transferred to solid rooting medium comprising MS basal salts, MS vitamins, 1.95 g/L MES, 40 g/L maltose, 0.5 mg/L cupric sulfate, 100 mg/L ascorbic acid, 1 mg/L IBA, 3 g/L Gelzan CM, 400 mg/L carbenicillin, 500 mg/L cefotaxime and 30 μM glyphosate, pH 5.8 and cultured at 25° C. with a photoperiod of 16 hours light/8 hours dark for 2 weeks. Putative transgenic wheat plants with developed roots were transplanted in soil plugs (2″ diameter×3″ height (Gro-Tech, Rough and Ready, CA 95975) for further growth and development.

### Example 3

### Elevated Temperatures During Bud Induction Improve Transformation of Corn Seed Explants

This example provides experiments designed to evaluate the effect of elevated temperatures during multiple bud induction on the transformation of corn seed excised explants.

### Comparison of Bud Induction for Two Weeks at 28° C. Vs One-Week at 35° C. Followed by One-Week at 28° C.

Explants excised from corn seeds can be placed into contact with bud induction medium and cultured at 28° C. for two weeks following Agrobacterium inoculation and co-culture.

To evaluate the effect of elevated temperature during the initial bud induction step on transformation, two temperature treatments were evaluated using glyphosate for selection: 1) bud induction for two weeks at 28° C.; and 2) bud induction for one week at an elevated temperature of 35° C. followed by one week of bud induction at 28° C. The proceeding and subsequent steps for transformation of the corn explants in this experiment were generally performed as described in Example 1.

Following Agrobacterium inoculation and co-culture, corn explants were transferred to bud induction medium containing MS salts and B5 vitamins supplemented with 2 mg/L TDZ, 1 mg/L 2,4-D and different concentrations of glyphosate and cultured under the temperature conditions described above. As demonstrated in Table 2, culturing the explants at 35° C. for the first week of bud induction appeared to inhibit explant germination, however, after the second week of bud induction at 28° C. the percentage of explants with green bud formation was increased. Furthermore, culturing the explants at an elevated temperature for the first week of bud induction resulted in a significant increase in the frequency of explants producing normal green shoots after 4 weeks of regeneration. This resulted in an increase in shoot frequency and a decrease in chimeric shoot frequency (Table 2). In addition, the results also demonstrated that percentage of normal shoots generally increased as the glyphosate concentration in the selection medium increased from 12.5 μM to 50 μM.

TABLE 2
Elevated temperature during the first week of bud
induction increases transgenic shoot regeneration.

| 1st Week | | | | Total | | Normal |
|---|---|---|---|---|---|---|
| Bud | Glyphosate | Total | Shoot | # | Normal | Shoot |
| Induction | Concentration | # | Frequency | Normal | Shoots | Frequency |
| Temperature | (μM) | Shoots | (%) | Shoots | (%) | (%) |
| 28° C. | 12.5 | 88 | 3.52 | 9 | 10.23 | 0.36 |
| | 25.0 | 22 | 0.88 | 7 | 31.82 | 0.28 |
| | 37.5 | 6 | 0.24 | 5 | 83.33 | 0.2 |
| | 50.0 | 6 | 0.24 | 4 | 66.67 | 0.16 |
| 35° C. | 12.5 | 194 | 7.76 | 28 | 14.43 | 1.12 |
| | 25.0 | 220 | 8.8 | 46 | 20.91 | 1.84 |
| | 37.5 | 149 | 5.96 | 34 | 22.82 | 1.36 |
| | 50.0 | 138 | 5.52 | 45 | 32.61 | 1.8 |

Total number shoots were calculated as the total number of shoots regenerated from inoculated explants, including both normal and chimeric shoots. The shoot frequency was calculated as the number of inoculated explants producing shoots divided by the total number of inoculated explants. Number normal shoots was calculated as the total number of normal shoots regenerated that did not show a chimeric tissue phenotype. The percentage normal shoots were calculated as the number of normal shoots divided by the total number of shoots. Normal shoot frequency was calculated as the number of normal shoots divided by the total number of inoculated explants.

In another experiment, various temperature conditions during the bud induction step were evaluated to compare: 1) 35° C. for the first week of bud induction followed by 28° C. for the second week of bud induction; and 2) two weeks of bud induction at 28° C. This experiment included two different batches of corn explants and different combinations of plant growth regulators in the bud induction media. The following plant growth regulator combinations were evaluated: 1) 2 mg/L TDZ and 1 mg/L 2,4-D; and 2) 10 mg/L BAP and 1 mg/L 2,4-D. As described above, the bud induction medium contained MS salts and B5 vitamins supplemented with either 2 mg/L TDZ, 1 mg/L 2,4-D or 10 mg/L BAP and 1 mg/L 2,4-D, and 5 μM glyphosate as a selection agent. Following Agrobacterium inoculation and co-culture as previously described, the explants were cultured according to the conditions described above. Following co-culture, there was no obvious difference in the transient expression between the two batches of explants. The results of this experiment confirmed that performing the first week of bud induction at 35° C. appears to initially slow the growth of the corn explants and make them more compact and greener compared to the explants cultured at 28° C. for the entire two weeks of bud induction. The results were independent of the explant batch or the plant growth regulator combination in the bud induction medium. The growth of the explants initially cultured at 35° C. recovered during the subsequent bud induction at 28° C. These explants produced a greater percentage of green explants compared to those cultured at 28° C. for two weeks after the explants were transferred to regeneration and selection media and grown for 2, 3 or 4 weeks. Furthermore, as demonstrated in Table 3, explants initially cultured at 35° C. demonstrated an increased shoot frequency and percentage of normal shoots, independent of the explant batch or the plant growth regulator combination in the bud induction medium.

TABLE 3
Elevated temperature during the first week of bud induction increased transgenic shoot
regeneration independent of explant batch or plant growth regulator combination.

| | 1st Week Bud | | | | Total | | Normal |
|---|---|---|---|---|---|---|---|
| | Induction | Bud | Total | Shoot | # | Normal | Shoot |
| Explant | Temperature | Induction | # | Frequency | Normal | Shoots | Frequency |
| Batch | (° C.) | Medium | Shoots | (%) | Shoots | (%) | (%) |
| 1 | 28 | 2 mg/L TDZ, | 47 | 1.88 | 9 | 19.15 | 0.36 |
| | | 1 mg/L 2,4-D | | | | | |
| | | 10 mg/L BAP, | 50 | 2.00 | 11 | 22.00 | 0.44 |
| | | 1 mg/L 2,4-D | | | | | |
| | 35 | 2 mg/L TDZ, | 77 | 3.08 | 16 | 20.78 | 0.64 |
| | | 1 mg/L 2,4-D | | | | | |
| | | 10 mg/L BAP, | 153 | 6.12 | 38 | 24.84 | 1.52 |
| | | 1 mg/L 2,4-D | | | | | |
| 2 | 28 | 2 mg/L TDZ, | 41 | 1.64 | 11 | 26.83 | 0.44 |
| | | 1 mg/L 2,4-D | | | | | |
| | | 10 mg/L BAP, | 69 | 2.76 | 52 | 24.64 | 0.68 |
| | | 1 mg/L 2,4-D | | | | | |
| | 35 | 2 mg/L TDZ, | 65 | 2.60 | 11 | 16.92 | 0.44 |
| | | 1 mg/L 2,4-D | | | | | |
| | | 10 mg/L BAP, | 182 | 7.28 | 44 | 24.18 | 1.76 |
| | | 1 mg/L 2,4-D | | | | | |

Total number shoots were calculated as the total number of shoots regenerated from inoculated explants, including both normal and chimeric shoots. The shoot frequency was calculated as the number of inoculated explants producing shoots divided by the total number of inoculated explants. Number normal shoots was calculated as the total number of normal shoots regenerated that did not show a chimeric tissue phenotype. The percentage normal shoots were calculated as the number of normal shoots divided by the total number of shoots. Normal shoot frequency was calculated as the number of normal shoots divided by the total number of inoculated explants.

### Comparison of Bud Induction at 28° C. Vs. 35° C. Followed by Extended Bud Induction

Three independent experiments were performed using three different batches of corn explants, to evaluate bud induction at 35° C. for the first week followed by 28° C. for the second week compared to bud induction for two weeks at 28° C. prior to the additional extended bud induction, selection, and regeneration steps as described above. Each experiment included four temperature conditions as provided in Table 4.

TABLE 4
Culturing explants at an elevated temperature during bud induction
dramatically increased transgenic shoot regeneration.

| | | | Extended | | |
|---|---|---|---|---|---|
| | Bud | Bud | Bud | | |
| | Induction | Induction | Induction | | |
| Experiment | Temperature | Duration | Temperature | Selection | NPF |
| Treatment | (° C.) | (week) | (° C.) | Medium | (%) |
| 1-1 (control) | 28 | 2 | — | Liquid | 0.40 |
| 1-2 | 35 + 28 | 1 + 1 | 28 | Solid | 1.40 |
| 1-3 | 35 + 28 | 1 + 1 | 28 | Liquid | 1.60 |
| 1-4 | 28 | 2 | 28 | Liquid | 0.28 |
| 2-1 (control) | 28 | 2 | — | Liquid | 0.39 |
| 2-2 | 35 + 28 | 1 + 1 | 28 | Solid | 1.29 |
| 2-3 | 35 + 28 | 1 + 1 | 28 | Liquid | 1.68 |
| 2-4 | 28 | 2 | 28 | Liquid | 0.68 |
| 3-1 (control) | 28 | 2 | — | Liquid | 0.60 |
| 3-2 | 35 + 28 | 1 + 1 | 28 | Solid | 1.25 |
| 3-3 | 35 + 28 | 1 + 1 | 28 | Liquid | 1.65 |
| 3-4 | 28 | 2 | 28 | Liquid | 1.44 |

As shown in Table 4, culturing explants at 35° C. for the 1st week of bud induction followed by culturing at 28° C. for a second week of bud induction dramatically increased transgenic shoot regeneration as compared to bud induction at 28° C. for two weeks in all three experiments. The normal plant frequency (NPF) was calculated as the number of normal plants divided by the total number of inoculated explants.

### Bud Induction at 35° C. For 4 Days Followed by Bud Induction at 28° C. For the Remaining Period Of Bud Induction was Sufficient to Increase Transgenic Shoot Regeneration as Compared to Bud Induction at 28° C. For 2 Weeks

The effect of various durations of bud induction at 35° C. on transformation was further evaluated. Four independent experiments were conducted with 2 weeks of bud induction at 28° C. (i.e., 0 days at 35° C.) serving as a control. The treatment conditions included bud induction at 35° C. for 2, 4 or 7 days before transferring to 28° C. for the remaining two week period (i.e., 2 days+12 days; 4 days+10 days; or 7 days+7 days). As shown in Table 5, explants cultured at 35° C. for 4 days during the first week of bud induction had a significant increase in transgenic shoot regeneration as compared to explants cultured at 28° C. throughout the bud induction step. In addition, the average shoot regeneration frequency and average normal shoot frequency for the bud induction treatment at 35° C. for 4 days was comparable to the bud induction treatment at 35° C. for 7 days, indicating that bud induction at an elevated temperature of 35° C. for 4 days is sufficient for the transformation improvement.

TABLE 5
Effect of 35° C. bud induction for various
durations on transgenic shoot regeneration frequency.

| Duration | | | Shoot | Normal |
|---|---|---|---|---|
| at 35° C. | # | Total # of | Frequency | Shoot |
| (Day) | Replication | Explants | (%) | Frequency (%) |
| 0 | 4 | 10,000 | 2.23 | 0.38 |
| 2 | 4 | 10,000 | 2.53 | 0.4 |
| 4 | 4 | 10,000 | 5.39 | 0.79 |
| 7 | 4 | 10,000 | 5.7 | 0.63 |

Shoot frequency was calculated as the number of explants producing shoots divided by the total number of explants on bud induction medium for the respective treatment, averaged over the four replicates for each treatment. Normal shoot frequency was calculated as the number of normal shoots divided by the total number of explants on bud induction medium for the respective treatment, averaged over the four replicates for each treatment.

### Example 4

### Regeneration Media Containing Low Levels of Salt Improves Corn Seed Explant Transformation

This example describes improvement in the transformation and rooting frequency of transgenic plants regenerated from corn excised explants contacted with media containing low levels of salt.

During the production of transgenic plants, it is desirable to directly transfer rooted transgenic shoots in contact with regeneration medium from the petri dish to soil, a process known as direct plugging, to eliminate the intermediate steps of growing plants or shoots in a Phytatray™ or PlantCon™ container. Direct plugging requires fewer resources, accelerates the transgenic plant production process, and improves transgenic plant quality. Corn explants can be regenerated by contacting the explants with MS regeneration media following the inoculation, co-culture, bud induction, and extended bud induction steps. The rooting frequency of shoots, however, is often low and variable when MS regeneration media is used, which impedes effective implementation of direct plugging. MS-based media contain high salt levels and therefore experiments were designed to evaluate whether other regeneration media, which contain lower salt levels, improve transformation and/or rooting frequency. The regeneration media evaluated were MS regeneration medium, B5 regeneration medium, Woody Plant Medium (WPM) regeneration medium, and a low nitrogen MS regeneration medium.

Agrobacterium comprising a transformation vector containing a chloroplast-targeted Agrobacterium aroA gene, an uidA gene, and flanking T-DNA borders were prepared as described in Example 1. The aroA gene is a selectable marker that confers resistance to glyphosate and the uidA gene encodes β-glucuronidase (GUS). Following Agrobacterium inoculation and co-culture, the corn explants were placed onto bud induction medium and cultured at 35° C. for one week followed by bud induction at 28° C. for an additional one week. The explants were then transferred to an extended bud induction medium and cultured for an additional two weeks before being placed onto either MS, B5, WPM, or a low nitrogen MS regeneration medium. The components of MS medium, B5 medium, WPM medium, and a low nitrogen MS medium are known in the art and are provided together in Table 1. As shown in Table 6, explants regenerated in contact with WPM regeneration medium demonstrated improved transformation as measured by the normal plant frequency (NPF) compared to explants regenerated in contact with MS regeneration medium.

TABLE 6
WPM regeneration medium improves the normal
plant frequency of transgenic corn plants.

| | Total | Shoot | Total # | Normal Shoot | |
|---|---|---|---|---|---|
| Regeneration | # | Frequency | Normal | Frequency | NPF |
| Medium | Shoot | (%) | Shoots | (%) | (%) |
| MS | 131 | 5.2 | 57 | 2.28 | 1.84 |
| B5 | 113 | 4.5 | 38 | 1.52 | 1.24 |
| WPM | 185 | 7.4 | 74 | 2.96 | 2.68 |
| Low Nitrogen MS | 166 | 6.6 | 76 | 3.04 | 2.24 |

Total number shoots were calculated as the total number of shoots regenerated from inoculated explants, including both normal and chimeric shoots. The shoot frequency was calculated as the number of inoculated explants producing shoots divided by the total number of inoculated explants. Number normal shoots was calculated as the total number of normal shoots regenerated that did not show a chimeric tissue phenotype. The normal shoot frequency was calculated as the number of normal shoots divided by the total number of inoculated explants. The normal plant frequency (NPF) was calculated as the number of normal plants divided by the total number of inoculated explants.

In another experiment, a different batch of corn excised explants and Agrobacterium containing a different transformation vector construct were used to evaluate the transformation and/or rooting frequency of shoots regenerated in contact with WPM regeneration medium compared to MS regeneration medium. The transformation vector construct used in this experiment comprised the aroA expression cassette described above, another cassette encoding a gene of interest, and flanking T-DNA borders. This expression cassette did not comprise the uidA gene. After Agrobacterium inoculation and co-culture, the corn explants were placed onto bud induction medium and cultured at 35° C. for one week followed by bud induction at 28° C. for an additional one week. The explants were then transferred to an extended bud induction medium and cultured for two weeks before being placed onto either MS regeneration medium or WPM regeneration medium. Regenerated transgenic shoots were then either grown in a Phytatray™ before being transplanted to soil or directly transplanted from petri plates to soil (direct to plug (DTP)). As shown in Table 7 the WPM regeneration medium demonstrated improved transformation as measured by NPF. As shown in Table 8 transgenic shoots grown in Phytatrays™ and regenerated in contact with WPM regeneration medium had double the rooting frequency compared to those grown in Phytatrays™ and regenerated in contact with MS regeneration medium. Transgenic shoots regenerated in contact with WPM regeneration medium also produced rooted plants sooner compared to those regenerated in contact with MS regeneration medium, as demonstrated by the increased rooted shoots at 1^{st} pull (%).

TABLE 7
WPM regeneration medium improves normal plant
frequency of transgenic corn plants.

| | | Shoot | Normal | |
|---|---|---|---|---|
| Regeneration | # | Frequency | Shoot | NPF |
| Medium | Explant | (%) | Frequency (%) | (%) |
| MS medium | 2,500 | 4.0 | 1.56 | 1.24 |
| WPM medium | 2,500 | 5.0 | 2.64 | 1.72 |
| MS medium (DTP) | 2,500 | 5.3 | N/A | 1.84 |
| WPM medium (DTP) | 2,500 | 6.6 | N/A | 2.76 |

The shoot frequency was calculated as the number of inoculated explants producing shoots divided by the total number of inoculated explants. The normal shoot frequency was calculated as the number of normal shoots divided by the total number of inoculated explants. The normal plant frequency (NPF) was calculated as the number of normal plants divided by the total number of inoculated explants.

TABLE 8
WPM regeneration media improves rooting
frequency of transgenic corn plants.

| | Rooted Shoots | Rooted Shoots | |
|---|---|---|---|
| Regeneration | at 1^{st} | at 2^{nd} | Rooted Shoots |
| Medium | Pull (%) | Pull (%) | Overall (%) |
| MS medium | 0 | 30 | 21 |
| WPM medium | 27 | 67 | 58 |

Rooted Shoots at 1^{st} Pull was calculated as the number of shoots with roots divided by total number of shoots at the first pull. Rooted Shoots at the 2^{nd} Pull (%) was calculated as the number of shoots with roots divided by total number of shoots at the 2^{nd} pull. Rooted Shoots Overall was calculated as the total number of shoots with roots divided by total number shoots from both the 1^{st} and 2^{nd} pulls.

A close examination of the macronutrients in MS, B5, WPM, and a low nitrogen MS regeneration media demonstrates that the WPM regeneration medium contains a low level of nitrogen and other salts compared to the other regeneration media evaluated (Table 9). The WPM regeneration medium contains about ⅕ of the total nitrogen compared to MS regeneration medium, about ½ the total salt compared to B5 regeneration medium, and about ⅓ of the total salt compared to the low nitrogen MS regeneration medium. In addition, WPM regeneration medium comprises about ⅓ of the potassium compared to MS regeneration medium and B5 regeneration medium and about ¼ of the potassium compared to low nitrogen MS regeneration medium. The results provided herein therefore demonstrate that the lower salt levels present in WPM regeneration medium likely result in the observed improved transformation and rooting frequency of transgenic corn plants regenerated in contact therewith.

TABLE 9
Macronutrient concentrations in MS, B5, WPM,
and a low nitrogen MS regeneration media.

Salt Concentration (mM)

| Salt Type | MS | B5 | WPM | Low Nitrogen MS |
|---|---|---|---|---|
| Total Nitrogen | 60.02 | 25.74 | 12.35 | 31.18 |
| NO₃ | 39.41 | 24.73 | 9.7 | 27.7 |
| NH₄ | 20.61 | 2.0 | 5.00 | 3.48 |
| SO₄ | 1.73 | 2.19 | 7.32 | 5.11 |
| PO₄ | 1.25 | 1.09 | 1.25 | 1.25 |
| K | 20.04 | 24.73 | 12.6 | 28.94 |
| Ca | 2.99 | 1.02 | 3.01 | 2.99 |
| Mg | 1.5 | 1.55 | 1.01 | 1.50 |

### Example 5

### Force-Assisted Transformation Improves Corn Explant Transformation

This example demonstrates that force-assisted transformation improves the transformation of corn embryo explants excised from dry seeds. In this example, the force-assisted transformation may comprise subjecting the explant to high pressure, centrifugation, or high pressure and centrifugation prior to or during inoculation with Agrobacterium.

Experiments were designed to evaluate the transformation of mature corn embryo explants from dry seeds subjected to centrifugation prior to or during inoculation with Agrobacterium. Explants were surface sanitized with 95% ethanol and 200 ppm active chlorine, rinsed 3 times, rehydrated in 20% PEG4000 INO medium comprising ⅖ strength of B5 macro salt, 1/10 strength of B5 micro salts and vitamins, 1 g/L potassium nitrate, 30 g/L dextrose, 3.9 g/L 2-(N-morpholino) ethanesulfonic acid (MES) and 20% PEG4000, pH 5.4 for 3 hours, rinsed, and floatation enriched prior to inoculation. The dry embryo explants were then subjected to one of the following treatments: 1) sonication for 1 min at 45 kHz followed by incubation for 30 minutes at room temperature in the presence of Agrobacterium inoculum; 2) centrifugation at 291×g for 30 minutes prior to Agrobacterium inoculation followed by sonication for 1 min at 45 kHz and incubation for 30 minutes at room temperature in the presence of Agrobacterium inoculum; 3) sonication for 1 min at 45 kHz followed by centrifugation at 291×g for 30 minutes at room temperature in the presence of Agrobacterium inoculum; 4) incubation for 30 minutes at room temperature in the presence of Agrobacterium inoculum; 5) centrifugation at 291×g for 30 minutes prior to Agrobacterium inoculation followed by incubation in the presence of Agrobacterium inoculum for 30 minutes at room temperature; or 6) centrifugation at 291×g for 30 minutes at room temperature in the presence of Agrobacterium inoculum. The Agrobacterium used for inoculation in this experiment comprised a plant transformation vector comprising three expression cassettes, one first encoding β-glucuronidase (GUS), the second encoding GFP and the third encoding aadA. The uidA (GUS) gene was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The gfp gene was under the control of enhanced 35S RNA promoter from CaMV, the 5′ untranslated leader of wheat major chlorophyll a/b-binding protein, an intron from the rice actin 1 gene, and the 3′ UTR of the wheat low molecular weight heat shock protein gene. The aadA gene was under the control of enhanced 35S RNA promoter, the 5′ untranslated leader of wheat major chlorophyll a/b-binding protein fused with the first intron from rice Actin 1, and the 3′ UTR of the nopaline synthase gene of Agrobacterium tumefaciens Ti plasmid. Centrifugation was performed using a Sorvall™ RC3BP centrifuge and a H6000A rotor (Thermo Fisher Scientific, Waltham, MA, USA).

Following inoculation, the explants were co-cultured for 5 days at 23° C. and 70% relative humidity with a photoperiod of 16 hours light/8 hours dark in 1.25 ml INO media comprising ⅖ strength, 1/10 strength of B5 micro salts and vitamins, 1 g/L potassium nitrate, 30 g/L dextrose, 3.9 g/L 2-(N-morpholino) ethanesulfonic acid (MES), pH 5.4, plus 50 ppm nystatin, 10 ppm thiabendazole (TBZ), and 50 ppm pentachloronitrobenzene (PCNB). Explants were then sampled and analyzed for GUS activity using a quantitative MUG assay. As shown in Table 10, explants centrifuged either prior to or during inoculation demonstrated a significant increase in overall transient GUS expression. In this experiment, sonication did not have a statistically significant impact on transient GUS expression. There was also no statistically significant difference in transient GUS expression observed between explants centrifuged prior to Agrobacterium inoculation and those centrifuged during Agrobacterium inoculation. In these experiments, a negative GUS expression measurement is interpreted as no GUS staining.

TABLE 10
Improved Transient Expression in Explants Centrifuged
either Prior to or During Inoculation.

| | | Mean GUS | |
|---|---|---|---|
| | | Expression | |
| | | (pmol/μg | Standard |
| Treatment | # Replication | protein/hr) | Deviation |
| No centrifugation | 24 | −5.095 | 1.388 |
| Centrifugation at 291 × g | 24 | 0.837 | 2.282 |
| during inoculation | | | |
| Centrifugation at 291 × g | 24 | 0.903 | 2.199 |
| prior to inoculation | | | |

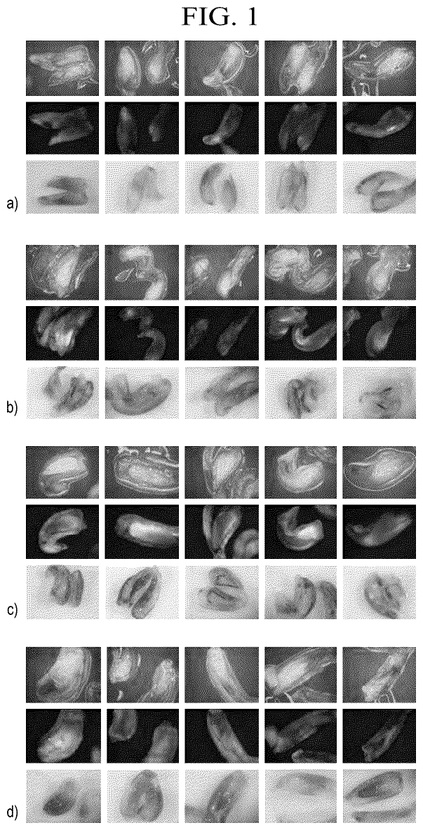

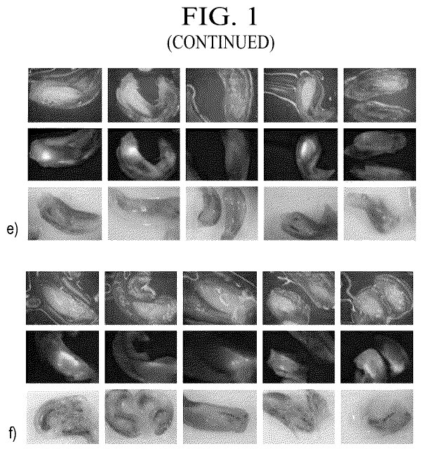

In addition, five explants from each treatment group were bisected and imaged for GFP and GUS expression. As shown in FIG. 1, transient expression was higher in the coleoptile and leaf base of explants centrifuged either before or during inoculation. FIG. 1 shows brightfield images, fluorescent images, and X-gal staining of explants subjected to: a) sonication for 1 min at 45 kHz followed by incubation for 30 minutes at room temperature in the presence of Agrobacterium inoculum; b) centrifugation at 291×g for 30 minutes prior to Agrobacterium inoculation followed by sonication for 1 min at 45 kHz and incubation for 30 minutes at room temperature in the presence of Agrobacterium inoculum; c) sonication for 1 min at 45 kHz followed by centrifugation at 291×g for 30 minutes at room temperature in the presence of Agrobacterium inoculum; d) incubation for 30 minutes at room temperature in the presence of Agrobacterium inoculum; e) centrifugation at 291×g for 30 minutes prior to Agrobacterium inoculation followed by incubation in the presence of Agrobacterium inoculum for 30 minutes at room temperature; or f) centrifugation at 291×g for 30 minutes at room temperature in the presence of Agrobacterium inoculum.

Experiments were designed to evaluate the transformation of mature corn embryo explants excised from dry seed subjected to centrifugation during Agrobacterium inoculation at 4° C. or 23° C. Explants were surface sanitized with 95% ethanol and 200 ppm active chlorine, rinsed 3 times, rehydrated in 20% PEG4000 in INO medium comprising ⅖ strength of B5 macro salts, 1/10 strength of B5 micro salts and vitamins, 1 g/L potassium nitrate, 30 g/L dextrose, 3.9 g/L 2-(N-morpholino) ethanesulfonic acid (MES), 20% PEG4000, pH 5.4 for 3 hours, rinsed, and floatation enriched prior to inoculation. The dry embryo explants were then subjected to one of the following treatments: 1) sonication for 1 min at 45 kHz followed by incubation for 30 minutes at 23° C. in the presence of Agrobacterium inoculum; 2) sonication for 1 min at 45 kHz followed by incubation for 30 minutes at 4° C. in the presence of Agrobacterium inoculum; 3) sonication for 1 min at 45 kHz followed by centrifugation at 291×g for 30 minutes at 23° C. in the presence of Agrobacterium inoculum; 4) sonication for 1 min at 45 kHz followed by centrifugation at 291×g for 30 minutes at 4° C. in the presence of Agrobacterium inoculum; 5) incubation for 30 minutes at 23° C. in the presence of Agrobacterium inoculum; 6) incubation for 30 minutes at 4° C. in the presence of Agrobacterium inoculum; 7) centrifugation at 291×g for 30 minutes at 23° C. in the presence of Agrobacterium inoculum; or 8) centrifugation at 291×g for 30 minutes at 4° C. in the presence of Agrobacterium inoculum. The Agrobacterium used for inoculation in this experiment comprised a plant transformation vector comprising three expression cassettes, one first encoding β-glucuronidase (GUS), the second encoding GFP, and the third encoding aadA. The uidA (GUS) gene was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The gfp gene was under the control of enhanced 35S RNA promoter from CaMV, the 5′ untranslated leader of wheat major chlorophyll a/b-binding protein, an intron from the rice actin 1 gene, and the 3′ UTR of the wheat low molecular weight heat shock protein gene. The aadA gene was under the control of enhanced 35S RNA promoter, the 5′ untranslated leader of wheat major chlorophyll a/b-binding protein fused with the first intron from rice Actin 1, and the 3′ UTR of the nopaline synthase gene of Agrobacterium tumefaciens Ti plasmid. Centrifugation was performed using a Sorvall™ RC3BP centrifuge and a H6000A rotor (Thermo Fisher Scientific, Waltham, MA, USA).

Following inoculation, the explants were co-cultured for 6 days at 23° C. and 70% relative humidity with a photoperiod of 16 hours light/8 hours dark in 1.25 ml INO media comprising ⅖ strength, 1/10 strength of B5 micro salts and vitamins, 1 g/L potassium nitrate, 30 g/L dextrose, 3.9 g/L 2-(N-morpholino) ethanesulfonic acid (MES), pH 5.4, plus 50 ppm nystatin, 10 ppm thiabendazole (TBZ), and 50 ppm pentachloronitrobenzene (PCNB). Explants were then analyzed for GUS activity using a quantitative MUG assay. As shown in Table 11, explants centrifuged during inoculation demonstrated a significant increase in overall transient GUS expression regardless of the temperature at which the inoculation was performed. In this experiment, neither incubation temperature nor sonication had a statistically significant impact on transient GUS expression. Similar to Table 10 above, a negative GUS expression measurement is interpreted as no GUS staining.

TABLE 11
Improved Transient Expression in Explants Centrifuged
During Inoculation at Various Temperatures.

| | | Mean GUS | |
|---|---|---|---|
| | # | Expression | Standard |
| Treatment | Replication | (pmol/μg protein/hr) | Deviation |
| No centrifugation | 12 | −0.7633 | 0.4423 |
| Centrifugation | 12 | 0.6575 | 0.5028 |
| at 291 g | | | |

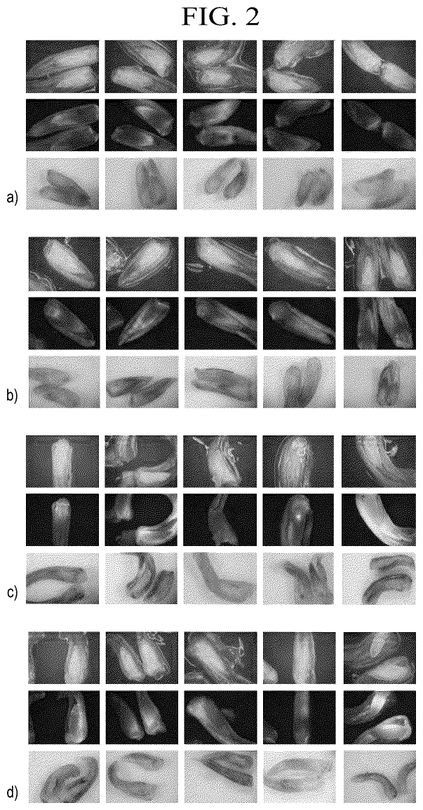

In addition, five explants from each treatment group were bisected and imaged for GFP and GUS expression. As shown in FIG. 2, transient expression was higher in the leaf base of explants centrifuged during inoculation. FIG. 2 shows brightfield images, fluorescent images, and X-gal staining of explants subjected to: a) sonication for 1 min at 45 kHz followed by incubation for 30 minutes at 23° C. in the presence of Agrobacterium inoculum; b) sonication for 1 min at 45 kHz followed by incubation for 30 minutes at 4° C. in the presence of Agrobacterium inoculum; c) sonication for 1 min at 45 kHz followed by centrifugation at 291×g for 30 minutes at 23° C. in the presence of Agrobacterium inoculum; d) sonication for 1 min at 45 kHz followed by centrifugation at 291×g for 30 minutes at 4° C. in the presence of Agrobacterium inoculum; e) incubation for 30 minutes at 23° C. in the presence of Agrobacterium inoculum; f) incubation for 30 minutes at 4° C. in the presence of Agrobacterium inoculum; g) centrifugation at 291×g for 30 minutes at 23° C. in the presence of Agrobacterium inoculum; or h) centrifugation at 291×g for 30 minutes at 4° C. in the presence of Agrobacterium inoculum.

Experiments were designed to evaluate transformation of mature corn embryo explants excised from dry seeds exposed to centrifugation or high pressure prior to inoculation with Agrobacterium. Explants were sanitized sequentially with 70% ethanol for 5 minutes and 10% Clorox®, rinsed 3 times, and rehydrated in inoculation medium comprising ⅖ strength of B5 macro salts, 1/10 strength of B5 micro salts and vitamins, 1 g/L potassium nitrate, 30 g/L dextrose, 3.9 g/L 2-(N-morpholino) ethanesulfonic acid (MES), pH 5.4, and 30 ppm Clearys® fungicide for 1 hour prior to the exposure to high pressure or centrifugation. Following exposure to high pressure or centrifugation, the dry embryo explants were inoculated with Agrobacterium comprising a plant transformation vector comprising two expression cassettes. One cassette comprises the uidA gene, which encodes β-glucuronidase (GUS), under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The other cassette comprises the aroA gene, which encodes class II EPSPS enzyme (5-enolpyruvylshikimate-3-phosphate synthase) and is targeted to the chloroplast by the transit peptide of Arabidopsis EPSPS, under the control of rice actin 1 promoter, leader and intron, and the 3′ UTR of the nopaline synthase gene of Agrobacterium tumefaciens Ti plasmid which functions to direct polyadenylation of the mRNA. for 1 hour at room temperature at various time points following exposure. Centrifugation was performed by placing explants in 50 ml polypropylene centrifuge tubes (Falcon® Bluemax™, Becton Dickinson 4-2098-11) with about 50 ml inoculation medium, as described above, and centrifuging using a Sorvall™ RC3BP centrifuge and a H6000A rotor (Thermo Fisher Scientific, Waltham, MA, USA). High pressure was applied by loading dry embryo explants in 20 ml of the inoculation medium described above into a French Press 40K pressure cell (Thermo® IEC, FA-032) and exposing the explants to 200 psig under high ratio for 5 minutes, which corresponds to a pressure of 3334 psi (227 atm) within the pressure cell.

Following inoculation, the explants were subjected to co-culture, bud induction, extended bud induction, and regeneration. Briefly, the explants were co-cultured with Agrobacterium for 5 days by plating on filter paper in a Plant Con® (MP Biomedicals, LLC catalog #26-720-02) growth chamber with 1.25 ml inoculation medium as described above comprising 30 ppm Clearys® and 5 ppm 2,4-D at 23° C. with a photoperiod of 16 hours light/8 hours dark. Co-cultured explants were transferred to solid bud induction medium comprising MS salts, B5 vitamins, 30 g/L sucrose, 0.69 g/L proline, 1 g/L NZ amine-A (casein enzymatic hydrolysate; Millipore Sigma), 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, and 3.5 g/L low EEO agarose, pH 5.8, and incubated for two weeks at 28° C. with a photoperiod of 16 hours light/8 hours of dark. Explants were then transferred to filter paper sandwiches for liquid selection at 28° C. with a photoperiod of 16 hours light/8 hours dark. During liquid selection, 10 ml of selection media comprising MS salts, B5 vitamins, 30 g/L sucrose, 0.69 g/L proline, 2 mg/L glycine, 1 g/L MES, 400 mg/L carbenicillin, 200 mg/L cefotaxime, 100 mg/L timentin, and 20 μM glyphosate, pH 5.8 was initially added to each plate and an additional 5 ml was added weekly. Shoots were transferred to bud induction and selection media comprising MS salts, B5 vitamins, 30 g/L sucrose, 0.69 g/L proline, 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, 20 μM glyphosate, and 3.5 g/L low EEO agarose, pH 5.8 8 to 10 weeks post-inoculation. Rooted R₀ events were sent to greenhouse for phenotyping and copy number analysis.

As shown in Table 12, exposure to either high pressure or centrifugation prior to inoculation with Agrobacterium significantly increased the transformation frequency of corn dry embryo explants. Furthermore, transformation frequency declined as the elapsed time between exposure to high pressure or centrifugation and inoculation increased. Transformation frequency was calculated as the number of R₀ plants divided by the total number of explants transferred to bud induction media.

TABLE 12
Improved Transformation Frequency of Corn Explants Exposed
to Centrifugation or High Pressure prior to Transformation.

| | | | Time post- | | Estimated | Initial | Explants | | |
|---|---|---|---|---|---|---|---|---|---|
| Force Treatment: Time | Force | | Treatment | | Treatment | Explants | used in | R₀ | Transformation |
| post-Treatment inoculated | Treatment | | inoculated | | Force | inoculated | stables | plants | Frequency (%) |
| 30 min 1 g: 15 min | 1 | g | 15 | min | 0.5N | 1522 | 1387 | 0 | 0.0% |
| 30 min 1 g: 30 min | 1 | g | 30 | min | 0.5N | 1522 | 1387 | 0 | 0.0% |
| 30 min 1 g: 6 hr | 1 | g | 6 | hr | 0.5N | 1529 | 1394 | 0 | 0.0% |
| 30 min 1 g: 30 hr | 1 | g | 30 | hr | 0.5N | 1507 | 1372 | 0 | 0.0% |
| 30 min 654 g: 15 min | 654 | g | 15 | min | 320N | 1532 | 1397 | 45 | 3.2% |
| 30 min 654 g: 30 min | 654 | g | 30 | min | 320N | 1510 | 1375 | 34 | 2.5% |
| 30 min 654 g: 6 hr | 654 | g | 6 | hr | 320N | 1526 | 1391 | 38 | 2.7% |
| 30 min 654 g: 30 hr | 654 | g | 30 | hr | 320N | 1601 | 1466 | 36 | 2.5% |
| 30 min 2619 g: 15 min | 2619 | g | 15 | min | 1280N | 1551 | 1416 | 37 | 2.6% |
| 30 min 2619 g: 30 min | 2619 | g | 30 | min | 1280N | 1541 | 1406 | 49 | 3.5% |
| 30 min 2619 g: 6 hr | 2619 | g | 6 | hr | 1280N | 1535 | 1400 | 52 | 3.7% |
| 30 min 2619 g: 30 hr | 2619 | g | 30 | hr | 1280N | 1538 | 1403 | 31 | 2.2% |
| 5 min 227 atm: 15 min | 227 | atm | 15 | min | 13000N | 1598 | 1463 | 54 | 3.7% |
| 5 min 227 atm: 30 min | 227 | atm | 30 | min | 13000N | 1519 | 1384 | 37 | 2.7% |
| 5 min 227 atm: 6 hr | 227 | atm | 6 | hr | 13000N | 1573 | 1438 | 33 | 2.3% |
| 5 min 227 atm: 30 hr | 227 | atm | 30 | hr | 13000N | 1776 | 1641 | 17 | 1.0% |

Experiments were designed to evaluate the transformation of mature corn embryo explants exposed to high pressure prior to or during inoculation with Agrobacterium. In this experiment, corn dry embryo explants were not surface sanitized. High pressure was applied by loading dry embryo explants in either 1) 20 ml of inoculation medium comprising ⅖ strength of B5 macro salts, 1/10 of B5 micro salts and vitamins, 1 g/L potassium nitrate, 30 g/L dextrose and 3.9 g/L MES; or 2) 20 ml of Agrobacterium inoculum in inoculation medium into a French Press 40K pressure cell (Thermo® IEC, FA-032) and exposing the explants to a pressure ranging from 14.7 psi to 30,000 psi. Dry embryo explants were then inoculated for 30 minutes with Agrobacterium comprising a plant transformation vector comprising two expression cassettes. One cassette comprises the uidA gene, which encodes β-glucuronidase (GUS), under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The other cassette comprises the aroA gene, which encodes class II EPSPS enzyme (5-enolpyruvylshikimate-3-phosphate synthase) and is targeted to the chloroplast by the transit peptide of Arabidopsis EPSPS, under the control of the rice actin 1 promoter, leader and intron, and the 3′ UTR of the nopaline synthase gene of Agrobacterium tumefaciens Ti plasmid which functions to direct polyadenylation of the mRNA.

Following inoculation, the explants were subjected to co-culture, bud induction, extended bud induction, and regeneration. Briefly, the explants were co-cultured with Agrobacterium for 5 days by plating on filter paper in a Plant Con® (MP Biomedicals, LLC catalog #26-720-02) growth chamber with 1.25 ml inoculation medium as described above comprising 30 ppm Clearys® and 5 ppm 2,4-D at 23° C. with a photoperiod of 16 hours light/8 hours dark. Co-cultured explants were transferred to solid bud induction medium comprising MS salts, B5 vitamins, 30 g/L sucrose, 0.69 g/L proline, 1 g/L NZ amine-A (casein enzymatic hydrolysate; Millipore Sigma), 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, and 3.5 g/L low EEO agarose, pH 5.8, and incubated for two weeks at 28° C. with a photoperiod of 16 hours light/8 hours of dark. Explants were then transferred to filter paper sandwiches for liquid selection at 28° C. with a photoperiod of 16 hours light/8 hours dark. During liquid selection, 10 ml of selection media comprising MS salts, B5 vitamins, 30 g/L sucrose, 0.69 g/L proline, 2 mg/L glycine, 1 g/L MES, 400 mg/L carbenicillin, 200 mg/L cefotaxime, 100 mg/L timentin, and 20 μM glyphosate, pH 5.8 was initially added to each plate and an additional 5 ml was added weekly. Shoots were transferred to bud induction and selection media comprising MS salts, B5 vitamins, 30 g/L sucrose, 0.69 g/L proline, 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, 20 μM glyphosate, and 3.5 g/L low EEO agarose, pH 5.8 8 to 10 weeks post-inoculation. Rooted R₀ events were sent to greenhouse for phenotyping and copy number analysis. As shown, in Table 13 transformation frequency was significantly increased in explants exposed to 1667 psi, 3334 psi, 10,000 psi, and 20,000 psi of pressure. In this experiment, exposure to 30,000 psi appeared to be lethal. Transformation frequency was calculated as the number of R₀ plants divided by the total number of explants transferred to bud induction media.

TABLE 13
Improved Transient Expression in Explants Exposed
to High Pressure Prior to or During Inoculation.

| | Number Explants | | Transformation |
|---|---|---|---|
| Treatment | used in Stables | R₀ plants | Frequency (%) |
| Inoculation medium, | 117 | 0 | 0.0% |
| 14.7 psi | | | |
| Inoculation medium, | 25 | 2 | 8.0% |
| 1,667 psi | | | |
| Inoculation medium, | 37 | 3 | 8.1% |
| 3,334 psi | | | |
| Agrobacterium | 70 | 4 | 5.7% |
| inoculum, 1,667 psi | | | |
| Agrobacterium | 51 | 4 | 7.8% |
| inoculum, 3,334 psi | | | |
| Inoculation medium, | 58 | 6 | 10.3% |
| 10,000 psi | | | |

Experiments were designed to evaluate the transformation frequency of mature corn embryo explants from dry seeds subjected to centrifugation, high pressure, or a combination of centrifugation and high pressure during Agrobacterium inoculation. Two batches of explants were prepared as described in Example 1. Each explant batch was subjected to one of the following treatments: (1) centrifugation at 2,620×g for 30 minutes in the presence of Agrobacterium inoculum; (2) high pressure at 300 psi for 3 minutes in the presence of Agrobacterium inoculum; (3) high pressure at 300 psi for 3 minutes prior to inoculation followed by centrifugation at 2,620×g for 30 minutes in the presence of Agrobacterium inoculum; or (4) high pressure at 300 psi for 3 minutes in the presence of Agrobacterium inoculum followed by centrifugation at 2,620×g for 30 minutes in the presence of Agrobacterium inoculum. The third treatment, high pressure prior to inoculation followed by centrifugation in the presence of Agrobacterium inoculum, allowed for the contribution of the high pressure component in the combined steps of the fourth treatment group to be evaluated. Each of the two explant batches were divided into one of the four treatment groups. Three repetitions were completed for each treatment using 1,140 explants per repetition. High pressure was applied using a Model 600-EXP Pressure Chamber (PMS Instrument Company, Albany, OR, USA), which was connected to a larger external pressure chamber through the external port. Nitrogen gas was used to pressurize the chamber. Centrifugation was applied using a Sorvall™ RC3BP centrifuge (Thermo Fisher Scientific, Waltham, MA, USA).

The corn dry embryo explants were sterilized with a solution of polyethylene glycol (PEG) and ethanol for 4 minutes and then rehydrated with 40 ml of rehydration medium for two hours. Explants were then inoculated with Agrobacterium while being exposed to centrifugation, high pressure, or a combination of centrifugation and high pressure as described above. The Agrobacterium used for inoculation comprised a binary plant transformation vector with two expression cassettes, the first encoding β-Glucuronidase (GUS) and the second encoding an EPSPS-CP4. The initial rehydration and subsequent high pressure and centrifugation treatments were performed using 50 ml Corning® mini bioreactor tubes with vented caps (catalog number CLS431720, Sigma-Aldrich, St. Louis, MO, USA).

Following inoculation, the explants were subjected to co-culture, bud induction, extended bud induction, and regeneration as described herein. Briefly, the explants were co-cultured for 5 days in a Percival® growth chamber at 20° C. and 70% relative humidity with a photoperiod of 16 hours light/8 hours dark. Each co-culture tray holds up to approximately 570 explants, therefore two trays (or up to about 1,140 explants) were used per treatment repetition. Co-cultured explants from each tray were then transferred to 5 trays containing bud induction medium and incubated for one week at 33° C. and 70% relative humidity with a photoperiod of 16 hours light/8 hours of dark. Explants from each bud induction tray were then transferred to two trays of extended bud induction medium with 25 μM glyphosate for selection and incubated for one week at 33° C. and 70% relative humidity with a photoperiod of 16 hours light/8 hours of dark. Explants were transferred to regeneration medium in Vivi® trays (VIVI Green Innovators, ś-Gravendeel, The Netherlands) with 20 μM glyphosate selection and incubated for 4 to 6 weeks to allow transgenic plants to develop. Following regeneration, phenotypically normal plants were plugged into plugging medium (Product #72-R, Gro-Tech.com Falmouth, ME, USA) and incubated in trays (Item #PL-O36-STAR-HW-VH-BK-50, Clearwater, MN, USA). Leaf tissue samples for molecular assays were obtained from each plugged plant. Plugged plants were then moved to a growth chamber for hardening.

Based on molecular data, transformed plants were advanced to the greenhouse for observation. GUS expression was used as a visible marker to detect transient expression following co-culture and to detect stable expression at other time points, such as during bud induction and R₀ shoot development. GUS expression assays of R₀ leaf tissue and visual observation of plants at plugging was also used to identify chimeric plants. Chimeric plants typically had the phenotype of striped leaves with green and white sectors during plugging. Molecular assays were performed on R₀ leaf tissue using real-time PCR to determine the presence and copy number of the CP4 EPSPS and uidA genes. Based on these assays, the percentage of non-chimeric transformed plants (a.k.a. normal transgenic plants or normal plants) for each treatment was determined by dividing the number of non-chimeric, and both CP4 EPSPS and uidA positive transgenic plants by the total number of plants sampled after plugging. Table 14 shows the plugging frequency, the frequency of low copy number plants (i.e., the percentage of non-chimeric plants that were positive for a low copy number of both the EPSPS CP4 gene and the uidA gene), and frequency of transformants with low copy number transgenes. The plugging frequency was calculated as the number of transgenic R₀ plants plugged divided by the total number of inoculated explants. The frequency of low copy number plants was calculated as the number of low copy number transgene-positive (i.e., plants with only one or two copies of each transgene) and non-chimeric plants divided by the total number of R₀ plants plugged. The frequency of transformants with low copy number transgene was calculated as the number of transformants with low copy number transgenes divided by the total number of inoculated explants. Each of these frequency calculations was further multiplied by 100% to express the frequency as a percentage. The calculations were made based on samples taken from a random number of plants after plugging.

TABLE 14
Improved Transformation Frequency in Corn Explants Exposed
to a Combination of High Pressure and Centrifugation.

| | | | Frequency of |
|---|---|---|---|
| | | Frequency of | Transformants |
| | | Low Copy | with Low Copy |
| | Plugging | Number | Number |
| Treatment | Frequency | Plants | Transgene |
| Treatment 1 | 2.2% | 53.0% | 1.2% |
| Centrifugation | | | |
| Treatment 2 | 3.0% | 53.4% | 1.6% |
| (High Pressure) | | | |
| Treatment 3 | 1.9% | 53.8% | 1.0% |
| (High Pressure, | | | |
| no Agro + Centrifugation) | | | |
| Treatment 4 | 3.0% | 65.6% | 2.0% |
| (High pressure + | | | |
| Centrifugation) | | | |

As shown in Table 14, the frequency of low copy number plants and frequency of transformants with low copy transgene were highest when inoculation was performed using high pressure at 300 psi for 3 minutes and centrifugation at 2,620×g for 30 minutes in the presence of Agrobacterium inoculum. The low copy transformation frequency was second highest when inoculation was performed using high pressure at 300 psi for 3 minutes in the presence of Agrobacterium inoculum. Therefore, the combination of centrifugation and high pressure during inoculation demonstrates an additional increase in transformation and low copy number frequency compared to inoculation using either high pressure or centrifugation alone.

### Example 6

### Force-Assisted Transformation Improves Wheat Explant Transformation

This example describes improvement in the transformation of wheat embryo explants excised from dry seeds using force-assisted transformation. In this example, the force-assisted transformation may comprise subjecting the explant to centrifugation during inoculation with Agrobacterium.

Explants were surface sanitized with 70% ethanol for 1 minute, rinsed, floatation enriched, rehydrated in 20% PEG4000 for 1.5 hours, and rinsed prior to inoculation. The dry embryo explants were subjected to one of the following treatments: 1) incubation for 30 minutes at 4° C. in the presence of Agrobacterium inoculum; 2) centrifugation at 72×g for 30 minutes at 4° C. in the presence of Agrobacterium inoculum; 3) centrifugation at 291×g for 30 minutes at 4° C. in the presence of Agrobacterium inoculum; or 4) centrifugation at 654×g for 30 minutes at 4° C. in the presence of Agrobacterium inoculum. The Agrobacterium used for inoculation in this experiment comprised a plant transformation vector comprising three expression cassettes, one first encoding β-glucuronidase (GUS), the second encoding GFP and the third encoding aadA. The uidA (GUS) gene was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The gfp gene was under the control of enhanced 35S RNA promoter from CaMV, the 5′ untranslated leader of wheat major chlorophyll a/b-binding protein, an intron from the rice actin 1 gene, and the 3′ UTR of the wheat low molecular weight heat shock protein gene. The aadA gene was under the control of enhanced 35S RNA promoter, the 5′ untranslated leader of wheat major chlorophyll a/b-binding protein fused with the first intron from rice Actin 1, and the 3′ UTR of the nopaline synthase gene of Agrobacterium tumefaciens Ti plasmid. Centrifugation was performed using a Sorvall™ RC3BP centrifuge and a H6000A rotor (Thermo Fisher Scientific, Waltham, MA, USA).

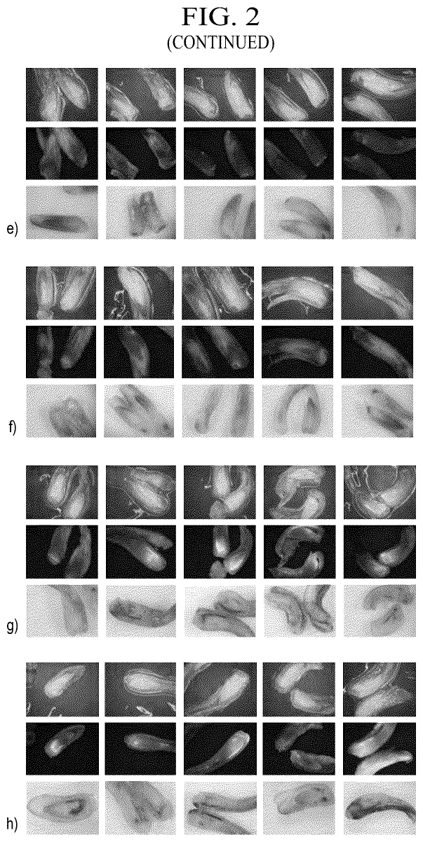

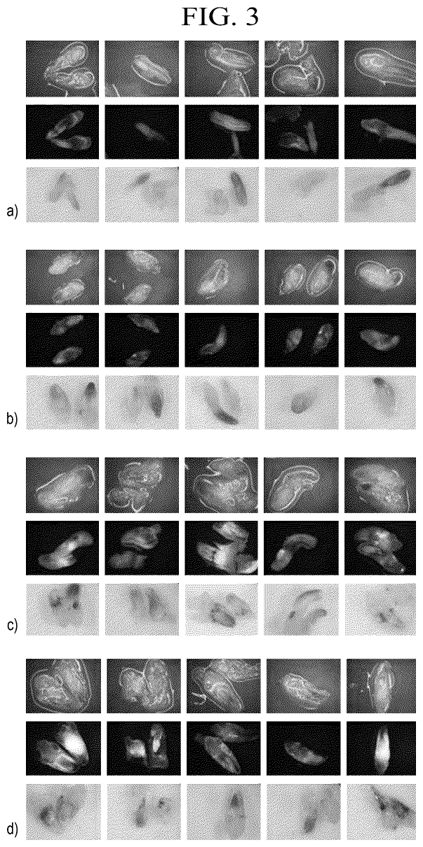

Following inoculation, the explants were co-cultured for 4 days at 23° C. and 70% relative humidity with a photoperiod of 16 hours light/8 hours dark in either 1.0 ml or 1.25 ml INO media comprising 50 ppm nystatin, 10 ppm thiabendazole (TBZ), and 50 ppm pentachloronitrobenzene (PCNB). Five explants from each treatment group were bisected and imaged for GFP and GUS expression a. As shown in FIG. 3, transient expression was increased in explants centrifuged at greater than 72×g during inoculation. FIG. 3 shows brightfield images, fluorescent images, and X-gal staining of explants subjected to: a) incubation for 30 minutes at 4° C. in the presence of Agrobacterium inoculum; b) centrifugation at 72×g for 30 minutes at 4° C. in the presence of Agrobacterium inoculum; c) centrifugation at 291×g for 30 minutes at 4° C. in the presence of Agrobacterium inoculum; or d) centrifugation at 654×g for 30 minutes at 4° C. in the presence of Agrobacterium inoculum.

### Example 7

### Effect of Gravitational Force and Centrifugation Container on Transformation of Corn Explants

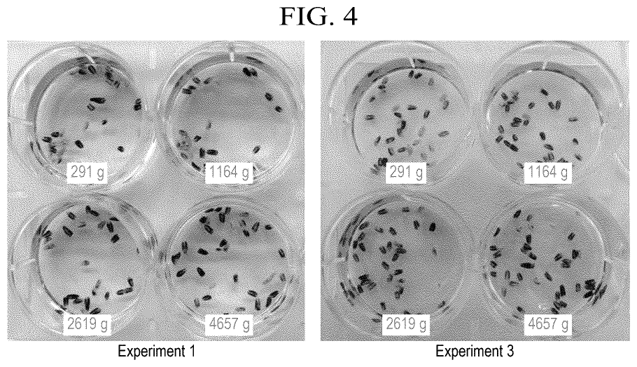

A gravitational force of 291×g or 654×g has been used to produce transgenic corn plants. Multiple experiments (Experiments 1, 2 and 3) were performed to determine if gravitational force during inoculation has a positive effect on transformation frequency (TF). As shown in FIG. 4 for two experiments (Experiments 1 and 3), transient expression was increased as gravitational force was increased from 291×g (or 1000 rpm in this experiment) to 4657×g (or 4000 rpm in this experiment). In addition, final transformation frequency was increased when gravitational force was increased from 291×g to 2619×g (or 3000 rpm in this experiment) but decreased somewhat as gravitational force was further increased to 4657×g probably due to the impact of the gravitational force on tissue culture response (see Table 15).

TABLE 15
Effect of gravitational force during
inoculation on stable transformation.

| | Putative TF % | | |
|---|---|---|---|
| Gravitational force (g) | Exp 1 | Exp 2 | Exp 3 |
| 291 | 0.13 | 0.08 | 0.4 |
| 1164 | 0.24 | 0.21 | 0.8 |
| 2619 | 0.32 | 0.37 | 2.44 |
| 4657 | 0.29 | 0.29 | 2.15 |

Falcon centrifuge tubes (50 ml) have been used for centrifugation during corn transformation, but Corning conical bottles (500 ml) have also been used to grow Agrobacterium in place of 50 ml Falcon centrifugation tubes with scale-up production implementation in mind. However, larger centrifugation containers may not be necessary with improved transformation frequencies. Multiple experiments (Experiments 1, 2, 3 and 4) were performed to investigate the effect of the centrifugation container on transformation frequency (TF). As shown in Table 16, greater TF was achieved with 50 ml Falcon tubes compared to 500 ml Corning conical bottles.

TABLE 16
Effect of centrifugation container on stable transformation.

| | |
|---|---|
| Centrifugation | Putative TF % |

| Container | Exp 1 | Exp 2 | Exp 3 | Exp 4 |
|---|---|---|---|---|
| 50 ml Falcon tube | 0.2 | 0.58 | 2.12 | 4.34 |
| 500 ml Corning | 0.06 | 0.26 | 0.36 | 0.58 |
| bottle | | | | |

### Example 8

### Rehydration of Corn Explants Prior to Agrobacterium Inoculation Improved Corn Explant Transformation

Two experiments were conducted to evaluate the effect of corn explant rehydration prior to Agrobacterium inoculation on corn explant transformation. Transformation was generally performed as described in Example 1, unless otherwise noted. Explants in each experiment were rehydrated for 0, 1, or 2 hours and each treatment group contained 3,333 corn explants.

Briefly, explants were surface sterilized using 70% ethanol for 4 min and then rinsed 3 times with sterile water prior to rehydration in medium containing ⅖ strength of B5 macro salts except for CaCl₂), which is ½ strength, 1/10 strength of B5 micro salts and vitamins, 1 g/L potassium nitrate, 30 g/L dextrose, 2.8 mg/L sequestrene, 3.9 g/L MES, 0.03 g/L Clearys 3336 WP, pH 5.4 and 5 mg/L 2,4-D for 0, 1, or 2 hours. In this experiment, the same medium used for rehydration was also used for Agrobacterium inoculation and co-culture. Explants were inoculated with Agrobacterium at an OD₆₀₀ of 0.35 with centrifugation at 600×g at 4° C. for 30 min. The Agrobacterium comprised two expression cassettes flanked by T-DNA borders on the Ti plasmid. The first expression cassette comprised a chloroplast-targeted CP4 EPSPS gene under the control of a constitutive promoter. The second expression cassette comprised a uidA gene under the control of a different constitutive promoter. Following co-culture, explants were cultured either in a warm room with a photoperiod cycle of 22 hours light/2 hours dark (Photoperiod 1) or in a warm room with a photoperiod cycle of 16 hours light/8 hours dark (Photoperiod 2).

As shown in Table 17, incorporation of rehydration step consistently improved shoot frequency and transformation frequency following culture with both photoperiods. Furthermore, rehydration for 2 hours improved shoot frequency and transformation frequency as compared to rehydration for 1 hour.

TABLE 17
Rehydration improves corn seed explant shoot
frequency and transformation frequency.

| | Rehydration | | # Shoots to | Shoot | | |
|---|---|---|---|---|---|---|
| | Duration | # Corn | Rooting | Frequency | # Plants | TF |
| Photoperiod | (hr) | Explants | Medium | (%) | Plugged | (%) |
| Photoperiod 1 | 2 | 3,333 | 101 | 3.03 | 51 | 1.5 |
| Photoperiod 1 | 1 | 3,333 | 35 | 1.05 | 13 | 0.4 |
| Photoperiod 1 | 0 | 3,333 | 3 | 0.09 | 1 | 0.0 |
| Photoperiod 2 | 2 | 3,333 | 91 | 2.73 | 75 | 2.3 |
| Photoperiod 2 | 1 | 3,333 | 30 | 0.9 | 24 | 0.7 |
| Photoperiod 2 | 0 | 3,333 | 10 | 0.3 | 9 | 0.3 |

The shoot frequency was calculated as the number of inoculated explants producing shoots divided by the total number of inoculated explants. Transformation frequency or TF (%) was calculated as the number of regenerated plants plugged divided by the total number of inoculated corn explants.

### Example 9

### Effect of Temperature and Light Intensity During Bud Induction on Corn Explant Transformation

Two experiments were conducted to evaluate the effect of temperature and light intensity during bud induction. Explants were surface sterilized, purified, rehydrated, and inoculated with Agrobacterium as described in Example 8. Explants were then co-cultured and subjected to bud induction, extended bud induction, and regeneration as described in Example 1, unless otherwise noted.

The first experiment evaluated bud induction at two culture temperatures, 32° C. and 35° C., and two light intensities of PAR, 90 μ/m²·s and 140-150 μ/m²·s, Each treatment group contained about 2,500 corn explants. Cultures were incubated in Percival® chambers at the designated temperature and light intensity. As shown in Table 18, performing bud induction at 32° C. improved shoot frequency, normal shoot frequency, number of normal shoot to soil plug, and normal plant frequency compared to performing bud induction at 35° C. In this experiment, light intensity did not have a statistically significant effect on normal plant frequency, however, performing bud induction at 90 μ/m²·s of PAR produced a slightly higher normal plant frequency compared to performing bud induction at 140-150 μ/m²·s of PAR.

TABLE 18
Effect of temperature and light intensity during
bud induction on corn explant transformation.

| | PAR | | | Normal | | |
|---|---|---|---|---|---|---|
| | Light | | Shoot | Shoot | # Normal | |
| Temperature | Intensity | # | Frequency | Frequency | Shoot to | NPF |
| (° C.) | (μ/m² · s) | Explants | (%) | (%) | Plug | (%) |
| 32 | 90 | 2495 | 5.7 | 5.1 | 44 | 1.8 |
| 32 | 140-150 | 2500 | 6.4 | 6.0 | 41 | 1.6 |
| 35 | 90 | 2374 | 2.3 | 3.6 | 20 | 0.8 |
| 35 | 140-150 | 2500 | 0.7 | 0.6 | 8 | 0.3 |

The shoot frequency was calculated as the number of inoculated explants producing shoots divided by the total number of inoculated explants. The normal shoot frequency was calculated as the number of normal shoots divided by the total number of inoculated explants. The normal plant frequency (NPF) was calculated as the number of normal plants divided by the total number of inoculated explants.

The second experiment evaluated bud induction at four different light intensities, 90 μ/m²·s of PAR, 150 μ/m²·s of PAR, 180 μ/m²·s of PAR, and 190 μ/m²·s of PAR. Bud induction was performed at 28° C. in the 180 PAR treatment group and 33° C. in the 90 μ/m²·s of PAR, 150 μ/m²·s of PAR, and 190 μ/m²·s of PAR treatment groups. Each treatment contained 2,500 corn explants. After Agrobacterium inoculation, the explants were transferred to co-culture medium comprising ⅖ of B5 macro salts, 1/10 of B5 micro salts and vitamins, 1 g/L potassium nitrate, 30 g/L dextrose, 3.9 g/L 2-(N-morpholino) ethanesulfonic acid (MES), 0.03 g/L Clearys 3336 WP, and 5 mg/L 2,4-D, pH 5.4. The explants were then transferred to bud induction medium and cultured in a Percival® chamber, except for the 180 μ/m²·s of PAR treatment group which cultured in a Conviron® chamber, at the designated temperatures and light intensities before extended bud induction and regeneration.

As shown in Table 19, bud induction at 33° C. generally improved shoot frequency, normal shoot frequency, number of normal shoot to soil plug, and normal plant frequency, as compared to bud induction at 28° C. In addition, bud induction at 90 μ/m²·s of PAR produced the highest normal plant frequency (NPF). In this experiment there was no significant different between bud induction at 150 μ/m²·s of PAR and bud induction at 190 μ/m²·s of PAR.

TABLE 19
Effect of temperature and light intensity during
bud induction on corn explant transformation.

| | PAR | | | Normal | | |
|---|---|---|---|---|---|---|
| | Light | | Shoot | Shoot | # Normal | |
| Temperature | Intensity | # | Frequency | Frequency | Shoot to | NPF |
| (° C.) | (μ/m² · s) | Explants | (%) | (%) | Plug | (%) |
| 33 | 90 | 2,500 | 7.56 | 3.36 | 52 | 3.3 |
| 33 | 150 | 2,500 | 5.96 | 1.72 | 25 | 2.0 |
| 33 | 190 | 2,500 | 7.36 | 2.20 | 26 | 2.2 |
| 28 | 180 | 2,500 | 4 | 2.12 | 20 | 1.6 |

The shoot frequency was calculated as the number of inoculated explants producing shoots divided by the total number of inoculated explants. The normal shoot frequency was calculated as the number of normal shoots divided by the total number of inoculated explants. The normal plant frequency (NPF) was calculated as the number of normal plants divided by the total number of inoculated explants.

### Example 10

### Effect of Light Density and Temperature During Regeneration on Transformation Frequency

To test whether light density has a significant effect on transformation efficiency in a liquid culture system, an experiment was conducted to evaluate the effect of light density on mature corn embryo explant transformation during the regeneration step. In this experiment, four petri dishes of liquid culture with about 30 explants per dish were stacked and placed into plastic boxes. Half of the boxes were cultured in a first light chamber with a light density of about 60 μmole/m²s and the other half were cultured in a second light chamber with a light density of about 100 μmole/m²s. The results showed that the transformation frequency was greater for regeneration in the presence of a higher light density of about 100 μmole/m²s compared with a lower light density of about 60 μmole/m²s. In each light room, transformation frequency was greatest for the petri dishes on the top of the boxes and stacks and gradually reduced from the top layers to the bottom layers as the light density gradually decreased (Table 20). Incidentally, the temperature was also highest with plates on the top layer and gradually reduced from the top layers to the bottom layers of plates (Table 21). Thus, a combination of higher light density and plate temperature may contribute to higher transformation frequencies. The temperature outside of the sweater boxes was about 29° C. Higher light density was also shown to increase transformation frequency for both liquid and solid culture protocols, but the magnitude of the increase was greater with the liquid culture than the solid culture protocol. In addition, higher light density was shown in these experiments to increase the rooting frequency of transgenic plants (Table 22).

TABLE 20
Effect of light density on stable transformation
with liquid culture protocol.

| Light density | | | | |
|---|---|---|---|---|
| (μmole/m²s | Position of | # Visible | | |
| PPFD) | petri dish | Explants | # Shoot pulled | Putative TF % |
| 60 (top layer) | Top layer | 625 | 14 | 2.24 |
| | 2^{nd} layer | 625 | 7 | 1.12 |
| | 3^{rd} layer | 625 | 8 | 1.28 |
| | 4^{th} layer | 625 | 6 | 0.98 |
| 100 (top layer) | Top layer | 625 | 43 | 6.88 |
| | 2^{nd} layer | 625 | 28 | 4.48 |
| | 3^{rd} layer | 625 | 22 | 3.52 |
| | 4^{th} layer | 625 | 12 | 1.92 |

TABLE 21
Correlation between actual plate light
density and temperature of plate.

| Position of | Light density (μmole/m²s | |
|---|---|---|
| petri dish | PPFD) | Plate temperature (° C.) |
| Top layer | 95 | 35 |
| 2^{nd} layer | 39 | 34 |
| 3^{rd} layer | 33 | 33 |
| 4^{th} layer | 32 | 32 |

TABLE 22
Effect of light density on liquid culture versus solid culture
and the frequency of rooting of transgenic plants.

| | | Putative TF % | | Rooting frequency % | |
|---|---|---|---|---|---|
| | | 60 | 100 | 60 | |
| Culture | | μmole/m²s | μmole/m²s | μmole/m²s | 100 μmole/m²s |
| method | Exp | PPFD | PPFD | PPFD | PPFD |
| Solid | 1 | 1.6 | 2.1 | 30.4 | 57.9 |
| | 2 | 1.6 | 1.9 | 33.5 | 68.8 |
| | 3 | 3.5 | 3.7 | 37.3 | 68.5 |
| | Average | 2.2 | 2.6 | 33.7 | 65.1 |
| Liquid | 1 | 1.4 | 4.2 | 31.4 | 41.9 |
| | 2 | 1.6 | 2.9 | 16.3 | 41.2 |
| | 3 | 2.1 | 3.7 | 17.1 | 36.9 |
| | Average | 1.7 | 3.6 | 21.6 | 40.0 |

### Example 11

### Effect of Different Cytokinins During Bud Induction on Corn Explant Transformation

An experiment was conducted to evaluate the effect of using different cytokinins during bud induction on corn explant transformation. Corn explants were surface sterilized, purified, rehydrated, inoculated with Agrobacterium, co-cultured, and subjected to the bud induction (without the second extended bud induction step) and regeneration steps as described in Example 1, unless otherwise noted. Glyphosate was used as the selection agent.

In this experiment, bud induction using four cytokinins, 6-Benzylaminopurine (BAP), Zeatin, thidiazuron (TDZ) and 6-(γ,γ-dimethylallylamino) purine (2iP), was evaluated. As shown in Table 23, bud induction using BAP at 10 mg/L or TDZ at 2 mg/L demonstrated significantly higher normal shoot frequencies compared to bud induction using 2 iP at 10 mg/L or zeatin at 2 mg/L. In this experiment, bud induction using TDZ produced a slightly higher normal shoot frequency compared to bud induction using BAP. In other studies that utilized, for instance, 1-8 mg/L BAP bud induction was also observed.

TABLE 23
Effect of different cytokinins during bud
induction on corn explant transformation.

| | | | | | | Normal |
|---|---|---|---|---|---|---|
| | | | Total | Shoot | # | Shoot |
| | Concentration | # | # | Frequency | Normal | Frequency |
| Cytokinin | (mg/L) | Explants | Shoot | (%) | Shoot | (%) |
| BAP | 10 | 5,000 | 290 | 5.80 | 78 | 1.56 |
| 2iP | 10 | 5,000 | 7 | 0.14 | 3 | 0.06 |
| Zeatin | 2 | 5,000 | 10 | 0.20 | 3 | 0.06 |
| TDZ | 2 | 5,000 | 444 | 8.88 | 85 | 1.70 |

The shoot frequency was calculated as the number of inoculated explants producing shoots divided by the total number of inoculated explants. The normal shoot frequency was calculated as the number of normal shoots divided by the total number of inoculated explants.

### Example 12

### Co-Culture without 2,4-D and Silwet® Improves Agrobacterium-Mediated Transformation of Wheat Seed Excised Explants

In this experiment, the effect of 2,4-D and Silwet®, a surfactant, in the co-culture medium for Agrobacterium-mediated transformation of wheat seed excised explants was evaluated. Wheat explants were surface sterilized, purified, inoculated with Agrobacterium, co-cultured, and subjected to delay, selection, and regeneration as described in Example 2, unless otherwise noted.

In this experiment, the addition of 2,4-D and Silwet® did not have a statistically significant effect on transformation frequency or normal plant frequency. As shown in Table 24, however, transformation frequency and normal plant frequency decreased when co-culture medium containing both 2,4-D and Silwet® was used compared to co-culture medium containing neither 2,4-D or Silwet®. Co-culture medium containing only Silwet® resulted in a higher normal plant frequency compared to co-culture medium containing neither Silwet® nor 2,4-D, however, transformation frequency was the highest in the absence of Silwet® and 2,4-D.

TABLE 24
Effect of 2,4-D and Silwet ® in co-culture
medium on wheat explant transformation.

| | | | Normal Plant Frequency |
|---|---|---|---|
| | | Transformation Frequency | (%) ± Standard |
| 2,4-D | Silwet ® | (%) ± Standard Deviation | Deviation |
| + | + | 1.0 ± 0.9 | 19.0 ± 11.3 |
| − | + | 5.0 ± 1.0 | 31.4 ± 2.0 |
| − | − | 7.1 ± 1.6 | 25.3 ± 5.0 |

Transformation frequency was calculated as the number of inoculated explants producing transgenic plants divided by the total number of inoculated explants. The normal plant frequency was calculated as the number of normal plants divided by the total number of plants regenerated.

### Example 13

### Co-Culture without 2,4-D Improves Agrobacterium-Mediated Transformation of Corn Seed Excised Explants

Seventeen independent experiments were conducted to evaluate the effect of 2,4-D in the co-culture medium on corn explant transformation. Corn explants were surface sterilized, purified, rehydrated, inoculated with Agrobacterium, co-cultured, and subjected to bud induction, extended bud induction, and regeneration as described in Example 1, unless otherwise noted.

Agrobacterium containing the same Ti plasmid as described in Example 8 was used for all experiments, except for experiments 13-17, in which two different bacterial origins of replication in the Ti-plasmid were evaluated in the presence or absence of 2,4-D in co-culture medium. In these cases, the Ti plasmids carried the same two expression cassettes flanked by T-DNA borders, one comprising the chloroplast-targeted CP4 EPSPS gene under the control of a constitutive promoter and the other comprising the uidA gene under the control of a similar constitutive promoter.

Table 25 provides a summary of results from treatments with and without 2,4-D in the co-culture medium from the 17 experiments. While these experiments included additional variables apart from the presence or absence of 2,4-D, the results show that removal of 2,4-D from the co-culture medium consistently resulted in higher normal plant frequencies in 16 out of 17 experiments across different sites and with different laboratory researchers, independent of any additional parameters. The increase in normal plant frequency ranged from 1.1 to 3.0 fold with an average increase of 1.8 fold.

TABLE 25
Removal of 2,4-D from co-culture media
improves corn explant transformation.

| | Normal Plant Frequency (%) | | |
|---|---|---|---|
| Experiment | With 2,4-D (5 mg/L) | Without 2,4-D | Fold Increase |
| 1 | 1.40 | 3.60 | 2.6 |
| 2 | 1.00 | 2.90 | 2.9 |
| 3 | 1.56 | 1.72 | 1.1 |
| 4 | 1.10 | 1.24 | 1.1 |
| 5 | 1.24 | 1.44 | 1.2 |
| 6 | 1.48 | 2.76 | 1.9 |
| 7 | 0.20 | 0.48 | 2.4 |
| 8 | 0.60 | 0.88 | 1.5 |
| 9 | 2.40 | 5.50 | 2.3 |
| 10 | 1.70 | 3.50 | 2.1 |
| 11 | 4.20 | 1.80 | 0.4 |
| 12 | 1.50 | 3.80 | 2.5 |
| 13 | 1.70 | 3.10 | 1.8 |
| 14 | 0.40 | 0.60 | 1.5 |
| 15 | 0.70 | 1.20 | 1.7 |
| 16 | 0.80 | 3.90 | 4.9 |
| 17 | 0.40 | 1.20 | 3.0 |
| OVERALL: | 1.32 | 2.33 | 1.8 |

The normal plant frequency was calculated as the number of normal plants divided by the total number of inoculated explants.

### Example 14

### Effect of Co-Culture Duration on Corn Explant Transformation

To evaluate the effect of co-culture duration on corn explant transformation, corn seed excised explants were surface sterilized in 70% ethanol for 4 minutes. Following incubation in rehydration medium comprising ⅖ strength of B5 macro salts except for CaCl₂), which is ½ strength, 1/10 of B5 micro salts and vitamins, 1 g/L potassium nitrate, 30 g/L dextrose, 2.8 mg/L sequestrene, 3.9 g/L 2-(N-morpholino) ethanesulfonic acid (MES), and 0.03 g/L Clearys 3336 WP, pH 5.4 on a shaker at 100 rpm at room temperature for 2 hours, the explants were inoculated with Agrobacterium as described in Example 1. The inoculated explants were then transferred to co-culture plates and co-cultured for 5 or 7 days before they were subjected to bud induction, extended bud induction, and regeneration as described in Example 1, except in this experiment the regeneration medium contained MS salts, B5 vitamins, 30 g/L sucrose, 0.69 g/L proline, 2 mg/L glycine, 1 g/L MES, 400 mg/L carbenicillin, 200 mg/L cefotaxime, 100 mg/L timentin and 20 UM glyphosate, pH 5.8. Regenerated shoots were rooted on rooting medium containing MS salts, B5 vitamins, 30 g/L sucrose, 0.69 g/L proline, 2 mg/L glycine, 1 g/L MES, 3 g/L Gelzan CM, 0.25 mg/L IBA, 250 mg/L carbenicillin, and 50 μM glyphosate, pH 5.8.

As shown in Table 26, co-culture for 7 days produced a higher total number of transgenic shoots compared to co-culture for 5 days, however, a higher percentage of the shoots produced were chimeric. As a result, co-culture for 5-days yielded a higher frequency of normal plants.

TABLE 26
Comparison of co-culture duration (5 vs
7 days) on corn explant transformation.

| | | | Shoot | | Normal |
|---|---|---|---|---|---|
| Duration | | Total # | Frequency | # Normal | Plant |
| (Days) | # Explants | Shoots | (%) | Plants | Frequency (%) |
| 5 | 1219 | 138 | 11.32 | 55 | 4.5 |
| 7 | 1250 | 147 | 11.80 | 37 | 3.0 |
| 5 | 1188 | 161 | 13.50 | 59 | 5.0 |
| 7 | 1219 | 160 | 13.10 | 32 | 2.6 |
| 5 | 1250 | 101 | 8.00 | 26 | 2.1 |
| 7 | 1250 | 127 | 10.20 | 33 | 2.6 |

The shoot frequency was calculated as the number of inoculated explants producing shoots divided by the total number of inoculated explants. The normal plant frequency was calculated as the number of normal plants divided by the total number of inoculated explants.

In a separate experiment, co-culture durations of 4 days and 5 days were evaluated in combination with the presence or absence of 5 mg/L 2,4-D in the co-culture medium. Corn seed excised explants were surface sterilized, rehydrated and inoculated with an Agrobacterium suspension as described in the preceding experiment. The Agrobacterium contained a chloroplast-targeted CP4 EPSPS gene under the control of a constitutive promoter flanked by T-DNA borders on the Ti plasmid. The inoculated explants were transferred to co-culture plates with or without 5 mg/L 2,4-D and co-cultured for 4 or 5 days prior to bud induction, extended bud induction, and regeneration as described in the preceding experiment.

As shown in Table 27, an average normal plant frequency of 2.47% was obtained when 2,4-D was absent in co-culture medium, as compared to 0.73% when 2,4-D was present. This data is consistent with the results described in Example 13. Furthermore, co-culture for 4 days produced a higher normal plant frequency compared to co-culture for 5 days. Table 28 provides average calculations based on the data provided in Table 27.

TABLE 27
Effect of 2,4-D in co-culture and co-culture
duration on corn explant transformation.

| | | | | | | Normal |
|---|---|---|---|---|---|---|
| 2,4-D | | | | Shoot | # | Plant |
| in Co- | Duration | # | Total # | Frequency | Normal | Frequency |
| culture | (Days) | Explants | Shoots | (%) | Plants | (%) |
| − | 4 | 2132 | 45 | 2.1 | 29 | 1.4 |
| + | 4 | 2296 | 35 | 1.5 | 19 | 0.8 |
| − | 4 | 492 | 35 | 7.1 | 22 | 4.5 |
| − | 5 | 1148 | 27 | 2.4 | 17 | 1.5 |
| + | 4 | 1148 | 21 | 1.1 | 10 | 0.9 |
| + | 5 | 820 | 9 | 1.1 | 4 | 0.5 |

The shoot frequency was calculated as the number of inoculated explants producing shoots divided by the total number of inoculated explants. The normal plant frequency was calculated as the number of normal plants divided by the total number of inoculated explants.

TABLE 28
Removal of 2,4-D from co-culture media improves
corn explant transformation as demonstrated
by the average normal plant frequency (%).

| | 2,4-D Present in | 2,4-D Absent in |
|---|---|---|
| Duration (Days) | Co-Culture | Co-Culture |
| 4 | 0.85 | 2.95 |
| 5 | 0.5 | 1.50 |

The normal plant frequency was calculated as the number of normal plants divided by the total number of inoculated explants.

### Example 15

### Agrobacterium-Mediated Transformation of Setaria viridis Crushed Seeds

Setaria viridis (green foxtail millet) can be an attractive model plant system for studying C₄ grass biology and other agronomic traits due to several characteristics, such as small stature (10-30 cm), rapid life cycle (6-9 weeks), prolific seed production (˜13,000 seeds per plant), self-compatibility, small genome size (˜395 Mb), diploid genetics (2n=18), and simple growth requirements. Agrobacterium-mediated transformation methods have been reported for S. viridis including tissue culture-based and in planta floral dip methods. See, e.g., Nguyen et al., “Robust and reproducible Agrobacterium transformation system of C4 genetic model species Setaria viridis”, Front. Plant Sci. 13:281 (2020); and Van Eck, J., “The Status of Setaria viridis Transformation: Agrobacterium-mediated to Floral Dip”, Front. Plant Sci. 9:652 (2018). However, prior tissue culture based methods involve Agrobacterium transformation of seed-derived callus culture, which is labor-intensive and prone to somaclonal variation and off-types in plants. Non-tissue culture based floral dip methods involve exposure of immature inflorescences of S. viridis plants to Agrobacterium followed by recovery of mature seeds and identification of transgenic plants grown from those harvested seeds. However, in planta floral dip methods require growth chamber or greenhouse space for growing and maintaining the plants before and after Agrobacterium infection.

Described herein is a novel approach for Agrobacterium-mediated transformation of Setaria seeds. An initial attempt was made to develop an Agrobacterium-mediated transformation system using dry excised seed embryo explants from Setaria viridis seeds, similar to the excised corn and wheat embryo explant systems described in the Examples above. However, while potentially capable of being transformed and regenerated into plants, the excised Setaria seed embryos were very small and difficult to handle. Thus, alternative approaches were developed using whole or crushed Setaria viridis seeds for Agrobacterium-mediated transformation, instead of excised seed embryo explants. Transformed Setaria plants were cultured and regenerated into plants from the inoculated whole or crushed seeds using a bud induction and regeneration protocol similar to the systems and methods described herein for corn and wheat seed embryo explant culture and regeneration.

### Setaria Crushed Seed Preparation

Up to 10 ml of Setaria seeds (˜5-10 grams) were placed into a 50 ml Falcon tube containing 30 ml of 70% ethanol for sterilization. After shaking, the seeds were poured into a 200 ml to 400 ml disposable plastic beaker and incubated for 2 min. The ethanol was then removed with a 35 ml pipet. The total ethanol contacting time may preferably be controlled to less than 4 minutes for Setaria seeds. The seeds were then rinsed 3 times with ˜200 ml of sterile water. Alternatively, the seeds were sterilized with 10% bleach plus 0.1% Tween 20 for 3 minutes, followed by rinsing with sterile water 3 times. The sterilized seeds were soaked in sterile water for at least 3 hours to rehydrate and soften the seeds before the rolling and crushing step. The sterilized and rehydrated seeds were then transferred to a clean plastic sheet, spread into a single layer, and covered with a second plastic sheet. The seeds between the two sheet layers were then crushed by rolling a 1 L bottle or rolling pin until a majority of the seed coats were crushed or opened. The crushed seeds were then transferred into one or more 50 ml Falcon tubes.

### Agrobacterium Inoculum Preparation

Approximately 0.5 mL of thawed Agrobacterium glycerol stock was inoculated into 200 ml of liquid LB medium containing 50 mg/L spectinomycin and 30 mg/L gentamicin selection in a sterile 1 L flask. The flask was placed into an orbital shaker/incubator set to 200 rpm and cultured at 28° C. in the dark for 16-24 hours, or until the absorbance at 660 nm (OD₆₆₀) of the inoculum was within a range of 0.6-1.0. Following centrifugation of the overnight Agrobacterium culture at approximately 3,000 rpm or 2,620×g (Sorvall® 3B, 6000A rotor) at 4° C. for 25 minutes, the pellet was resuspended in an inoculation medium comprising ⅖ strength of B5 macro salts, 1/10 of B5 micro salts and vitamins, 1 g/L potassium nitrate, 30 g/L dextrose, and 3.9 g/L MES, 5 mg/L 2,4-D and 0.05% Silwet, to a final OD₆₆₀ of about 0.5. Even though an auxin (2,4-D) and surfactant was used in the inoculation medium, one or both of these components may alternatively be absent from the inoculation medium.

### Agrobacterium Inoculation and Co-Culture

About 30 ml of the Agrobacterium suspension from the Agrobacterium Inoculum Preparation step was added to the Falcon tubes with the crushed seeds from the Setaria Crushed Seed Preparation step. The Agrobacterium/crushed seed mixtures were then centrifuged in a Sorvall 5B at ˜690-1500×g at 4° C. for 30 minutes. Following centrifugation, the tubes were agitated to resuspend the crushed seeds, the resuspended crushed seeds were poured into a container, and most of the Agrobacterium suspension was removed by pipetting.

The inoculated crushed seeds (from about 200-300 of starting seeds) were transferred to a deep petri dish containing a piece of sterile Whatman #1 filter paper (82 mm) wetted with 1.25 ml of the inoculation medium described in Agrobacterium Inoculum Preparation step above, and spread evenly into a single layer. The co-culture plates were incubated at 23° C. and about 70% relative humidity with a photoperiod of 16 hours light/8 hours dark for 3-8 days.

### Selection and Shoot/Root Regeneration

Following co-culture, the filter paper with the crushed seeds was transferred onto a shoot/bud induction medium comprising MS basal salts, B5 vitamins, 2 mg/L glycine, 690 mg/L proline, 1 g/L casein hydrolysate, 30 g/L sucrose, 1 g/L MES, 1 mg/L 2, 4-D, 10 mg/L BAP, 200 mg/L carbenicillin, 100 mg/L ticarcillin, 200 mg/L cefotaxime, and 3.5 g/L agarose, pH 5.8, with or without 10 μM glyphosate selection. Although no auxin was use in the shoot/bud induction medium in these experiments, low amounts of auxin could alternatively be used. The culture plates were incubated in a Percival incubator set at 28° C. with a photoperiod of 16 hours light/8 hours dark. Swelling and elongating seed explants were transferred to either a first regeneration medium comprising MS basal salts, B5 vitamins, 2 mg/L glycine, 690 mg/L proline, 30 g/L sucrose, 1 g/L MES, 400 mg/L carbenicillin, 100 mg/L ticarcillin, 200 mg/L cefotaxime, 30 μM glyphosate, and 3.5 g/L agarose, pH 5.8; or a second regeneration medium comprising MS basal salts, MS vitamins, 690 mg/L proline, 30 g/L sucrose, 1 g/L MES, 400 mg/L carbenicillin, 100 mg/L ticarcillin, 200 mg/L cefotaxime, 30 μM glyphosate, and 3.5 g/L agarose, pH 5.8. In experiment 3, after 4 weeks on the first regeneration medium, green multiple shoots were subcultured onto the same regeneration medium with 50 μM glyphosate. Dark green shoots were observed on this medium 2-3 weeks after the subculture and were transferred to rooting medium (the same medium or with low level of an auxin such as about 0.1-0.2 mg/L IAA). Although an auxin may have been used in the rooting medium in some of these experiments, the rooting medium may not contain any auxin or cytokinin.

In a first transformation experiment (Experiment 1), crushed seeds were inoculated with Agrobacterium tumefaciens cells containing a first Ti plasmid construct comprising two expression cassettes, the first encoding β-glucuronidase (GUS) as a scorable marker, and the second encoding CP4 EPSPS as a selection marker. The uidA (GUS) gene contained the second intron from the LS1 (light inducible gene) of potato and was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The aroA gene encoding the CP4 EPSPS protein was under the control of the promoter, leader, and intron of the rice actin 1 gene, and the 3′UTR of the nopaline synthase gene of Agrobacterium tumefaciens Ti plasmid which functions to direct polyadenylation of the mRNA. The EPSPS protein was targeted to the chloroplast by a fused transit peptide from an Arabidopsis EPSPS gene. Two additional experiments (Experiments 2 and 3) were performed using Agrobacterium tumefaciens cells carrying a second Ti plasmid construct comprising the same two GUS and EPSPS expression cassettes as in the first Ti plasmid construct. Approximately 5 grams of Setaria seeds were used for each experiment.

Table 29 summarizes the experimental details and results from all three transformation experiments with crushed seeds. In Experiment 1, the crushed seeds were subjected to 1,500×g of centrifugal force during the inoculation step, co-cultured for 8 days, transferred to shoot/bud induction medium for about 21 days without selection, and then regenerated in the second regeneration medium with 30 μM of glyphosate selection for about 6 weeks. In Experiment 2, the crushed seeds were subjected to 800×g of centrifugal force during the inoculation step, co-cultured for 4 days, transferred to shoot/bud induction medium for about 17 days with 10 UM of glyphosate selection, and then regenerated in the first regeneration medium with 30 μM of glyphosate selection for about 5 weeks. In Experiment 3, the crushed seeds were subjected to 800×g of centrifugal force during the inoculation step, co-cultured for 8 days, transferred to shoot/bud induction medium for about 23 days with 10 μM of glyphosate selection, and then subjected to a first regeneration step in the first regeneration medium with 30 μM of glyphosate selection for about 4 weeks, followed by a second regeneration step in the first regeneration medium with 50 μM of glyphosate selection for about 3 weeks. One transgenic (GUS+) plant was obtained from Experiment 1, and three transgenic (GUS+) plants were obtained from Experiments 2 and 3.

TABLE 29
Summary of Agrobacterium-mediated transformation of crushed Setaria seeds.

| | | Centrifugal | Co-culture | Shoot Induction | | Regeneration | | |
|---|---|---|---|---|---|---|---|---|
| | | Force during | Duration | Glyphosate | Duration | Glyphosate | Duration | # GUS + |
| Expt | Construct | Inoculation (g) | (days) | Selection | (days) | Selection | (weeks) | Shoots |
| 1 | 1 | 1,500 | 8 | No | 21 | Second | 6 | 1 |
| | | | | | | Regeneration | | |
| | | | | | | Medium +30 μM | | |
| 2 | 2 | 800 | 4 | 10 μM | 17 | First | 5 | 2 |
| | | | | | | Regeneration | | |
| | | | | | | Medium +30 μM | | |
| 3 | 2 | 800 | 8 | 10 μM | 23 | 1) First | 4 | 1 |
| | | | | | | Regeneration | | |
| | | | | | | Medium +30 μM | | |
| | | | | | | 2) First | 3 | |
| | | | | | | Regeneration | | |
| | | | | | | Medium +50 μM | | |

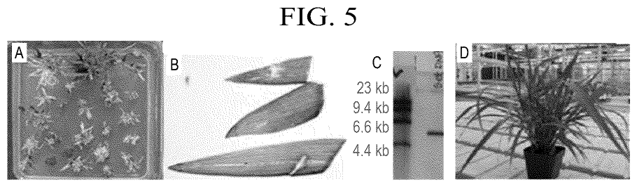

Images of an explant and regenerated plant with a transgenic insertion from the Experiment 1 is also shown in FIG. 5. Eight weeks after inoculation, a bushy shoot clump was obtained and confirmed by GUS staining (FIGS. 5A and 5B). The regenerated plants were grown in the greenhouse (FIG. 5D) and seeds were harvested. Southern blot hybridization showed that the event contained a single copy of the transgene when probed with CP4 (FIG. 5C).

### Example 16

### Agrobacterium-Mediated Direct Transformation of Setaria viridis Whole Seeds

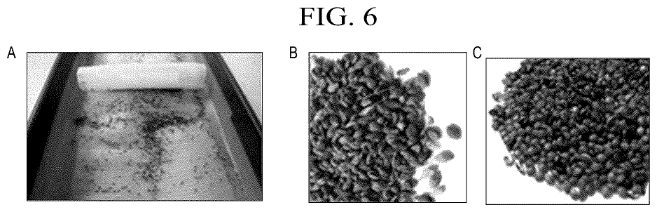

There are advantages to transforming whole or intact seeds without the need for crushing seeds or excising embryo explants. To test this possibility, whole or intact seeds were used for transformation with only their seed coats removed. The seed coats were removed from the Setaria seeds using a home-made polishing tool comprising a plastic drawer liner placed in the bottom of a tray and a paperboard core wrapped with another piece of ridged plastic liner. The Setaria seeds were placed on the liner in the tray and rolled over with the liner-covered paperboard core to loosen the seed coat mechanically (see FIG. 6A). FIG. 6B shows the Setaria seeds before removal of the seed coat, and FIG. 6C shows the Setaria seeds after removal or separation of the seed coats. The seeds were then poured into another container and blown with air to remove the seed coats, or the seeds were transferred into a water bath to allow for the seed to be separated from the seed coat as a result of the seed coat floating to the top of the water. Seeds were then collected for use in transformation.

After seed coat removal, the seeds were sterilized with 10% bleach plus 0.1% Tween 20 for 3 minutes, followed by rinsing with sterile water 3 times as described above in Example 15. The intact seeds were inoculated with Agrobacterium tumefaciens cells with the second Ti plasmid construct and cultured as described above for the crushed seeds in Example 15, except that the BAP concentration in the shoot/bud induction medium was 4 mg/L. Although no auxin was use in the shoot/bud induction medium in these experiments, low amounts of auxin could alternatively be used. Approximately 5 grams of Setaria seeds were used for each experiment. Plants were then regenerated from the inoculated and cultured seeds.

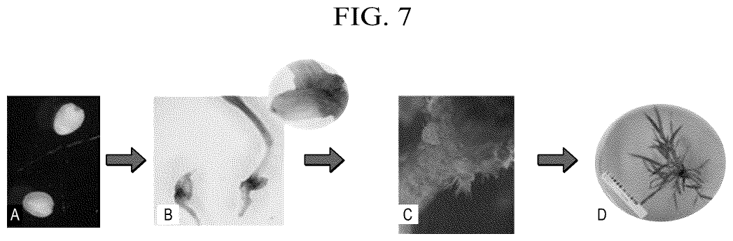

FIG. 7 shows the general progression of transformation and shoot regeneration after Agrobacterium infection of whole or intact seeds. Table 30 summarizes the experimental details and results from all three of these transformation experiments (Experiments 4, 5 and 6) with intact seeds. In Experiment 4, the seeds were subjected to 800×g of centrifugal force during the inoculation step, co-cultured for about 4 days, transferred to shoot/bud induction medium for about 8 days with 10 μM of glyphosate selection, and then regenerated in the first regeneration medium with 30 μM of glyphosate selection for about 6 weeks. In Experiment 5, the seeds were subjected to 1,500×g of centrifugal force during the inoculation step, co-cultured for 4 days, transferred to shoot/bud induction medium for about 10 days with 10 μM of glyphosate selection, and then regenerated in the first regeneration medium with 30 μM of glyphosate selection for about 5 weeks. In Experiment 6, the seeds were subjected to 1,500×g of centrifugal force during the inoculation step, co-cultured for 8 days, transferred to shoot/bud induction medium for about 10 days with 10 μM of glyphosate selection, and then subjected to a first regeneration step in the first regeneration medium with 30 μM of glyphosate selection for about 3 weeks, followed by a second regeneration step in the first regeneration medium with 50 μM of glyphosate selection for about 3 weeks. Two transgenic (GUS+) plant were obtained from Experiment 4, one transgenic (GUS+) plant was obtained from Experiment 5, and one transgenic (GUS+) plant was obtained in Experiment 6.

TABLE 30
Summary of Agrobacterium-mediated transformation of Setaria whole seeds.

| | | Centrifugal | Co-culture | Shoot Induction | | Regeneration | | |
|---|---|---|---|---|---|---|---|---|
| | | Force during | Duration | Glyphosate | Duration | Glyphosate | Duration | # GUS + |
| Expt | Construct | Inoculation (g) | (days) | Selection | (days) | Selection | (weeks) | Shoots |
| 4 | 2 | 800 | 4 | 10 μM | 8 | First | 6 | 2 |
| | | | | | | Regeneration | | |
| | | | | | | Medium +30 μM | | |
| 5 | 2 | 1,500 | 4 | 10 μM | 10 | First | 5 | 1 |
| | | | | | | Regeneration | | |
| | | | | | | Medium +30 μM | | |
| 6 | 2 | 1,500 | 8 | 10 μM | 10 | 1) First | 3 | 1 |
| | | | | | | Regeneration | | |
| | | | | | | Medium +30 μM | | |
| | | | | | | 2) First | 3 | |
| | | | | | | Regeneration | | |
| | | | | | | Medium +50 μM | | |

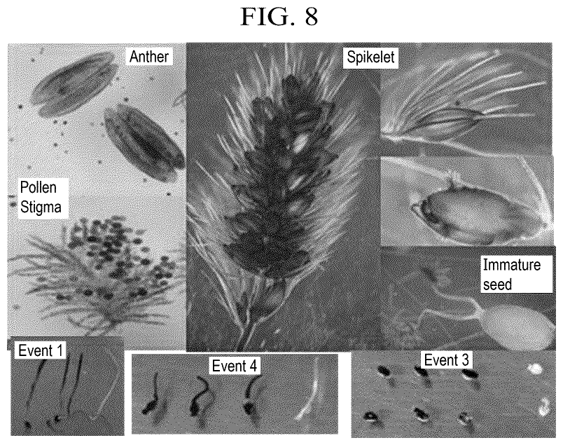

To confirm the transmission of the transgenes, R₀ reproductive tissues of the transformed plants and R₁ seedlings were stained for GUS expression. The staining results are shown in FIG. 8. GUS expression was observed in anthers, pollen stigma, spikelet, and immature seeds of transformed plants, as well as in germinated R₁ seedlings. R₁ seeds from two GUS-positive Setaria plant events were analyzed for segregation: Event 1 was from transformation of whole seeds according to the present Example 16, and Event 4 was from transformation of crushed seeds according to Example 15. Seedlings germinated from these R₁ seeds were stained for GUS expression. As shown in Table 31, both events showed an approximate 3:1 segregation, indicating that the transgene was integrated into a single locus.

TABLE 31
Transgene transmission in R1 progenies
of the transgenic Setaria plants.

| | | #GUS | #GUS | |
|---|---|---|---|---|
| | | Positive | Negative | Chi Square |
| | R₀ Phenotype | Seedlings | Seedlings | Test |
| Event 1 | normal | 225 | 79 | ~3:1 |
| Event 4 | normal | 111 | 41 | ~3:1 |

The results provided in Examples 15 and 16 support the use of whole or intact monocot seeds or crushed or partially opened monocot seeds for use in transformation and generation of genetically modified monocot plants through a multiple shoot/bud induction and regeneration process. It is demonstrated herein that transgenic Setaria viridis plants can be obtained by direct transformation of whole or crushed seeds using Agrobacterium-mediated transformation. The use of whole, intact, crushed or partially opened seeds could greatly facilitate the transformation of monocot plants by avoiding the need for embryo explant excision.

### Example 17

### Preparation of Flotation Media and Container

To test whether viable and regenerable explants in culture, following Agrobacterium inoculation, co-culture and bud induction steps, could be separated and sorted from undesirable material based on their flotation in a flotation medium inside a flotation vessel or container, corn seed embryo explants were added to a flotation medium following the extended bud induction step. The flotation medium in this experiment was prepared using sterile H₂O with 1 mg/L Absolute® 500 SC fungicide (Bayer Crop Science, St. Louis, MO 63167, USA) to control or prevent potential contamination. The flotation medium can be either premade in advance and stored (e.g., at 4° C.) for later use or prepared immediately prior to use. The medium was visually inspected for the absence of any observable contamination prior to use.

The flotation medium can be added to a suitable flotation vessel or container to a depth of about 2-3 inches of the flotation medium in the vessel. While the size of the floatation vessel and the amount or volume of flotation medium depend on the amount or number of cultured explants to be separated with a larger flotation vessel generally needed for separation of a greater amount or number of explants. The flotation vessels in the experiments described in Examples 18 and 19 were either (1) a 600 mL beaker that was filled with about 300 mL of flotation medium for smaller scale experiments, or (2) a ViVi® tissue culture vessel (Cat. #0901-0910; ViVi, Mijlweg 18, 3295 KH's-Gravendeel, The Netherlands) that was filled with about 2.5 L of flotation medium for larger scale experiments.

### Example 18

### Separation and Collection of Cultured Explants in Flotation Medium

For these experiments, embryo explants were excised from dry mature corn seeds. Prior to the flotation step, the excised embryo explants were subjected to Agrobacterium-mediated inoculation with a vector construct, co-culture, bud induction and extended bud induction steps as provided herein. After the extended bud induction step, cultured explants were added to the flotation medium contained in the flotation vessel for separation. The cultured explants can be added or transferred to the flotation medium in a variety of ways, such as by tapping on the culture plates or by using a utensil to transfer the explants, for example.

### Smaller Scale Experiments

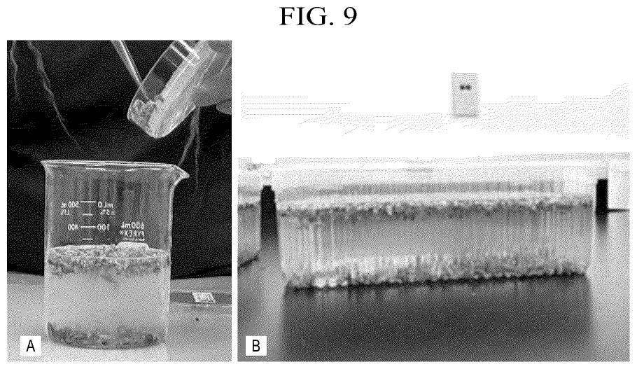

Four experiments were conducted. In these experiments, the seed-derived embryo explants were inoculated with Agrobacterium carrying a Ti plasmid, each containing two expression cassettes, one encoding an Agrobacterium CP4 EPSPS (5-enolpyruvylshikimate-3-phosphate synthase) driven by a constitutive promoter and targeted to the chloroplast, and the other encoding a different gene of interest for each construct in Experiments 1 to 3, or a beta-glucuronidase (uidA) driven by a root specific promoter in Experiment 4. Following extended bud induction, approximately 285 explants from five cultured plates were added to 300 mL of flotation medium in a sterile 600 mL beaker and gently agitated by swirling the beaker by hand for about 3-5 seconds. This volume of flotation medium had a depth of about 3 inches in the beaker. The explants were incubated in the flotation medium for about ten minutes at ambient temperature until two distinct layers of explant material formed in the flotation vessel including a top layer floating at or near the top of the flotation vessel and a bottom layer that sank toward the bottom of the vessel (see, e.g., FIG. 9A). It was observed that the top layer appeared to contain mostly viable greening explants with multiple bud formation, while the bottom layer contained mostly non-viable or dying explants that were brown or darker colored.

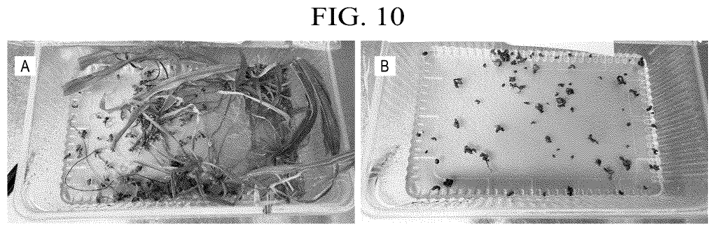

After a brief incubation of the explants in the flotation medium to allow for greater separation, the top floating layer of explants were collected by carefully decanting the flotation medium and top layer of explants into a metal mesh strainer. Explants collected in the strainer were blotted with sterile paper towel to remove excess floatation medium and transferred to a regeneration medium in Vivi® tissue culture vessels for the subsequent regeneration step. The non-floating explants in the bottom layer of the floatation vessel were also collected, blotted with sterile paper towel to remove excess medium, and placed onto regeneration medium in Vivi® tissue culture vessels, for comparison as described further below, in which the top layer and the bottom layer were tested side-by-side for regeneration and plugging (transfer to soil) frequencies. More healthy shoots and plants were regenerated from the explants of the top floating layer than the bottom non-floating layer (see, e.g., FIG. 10).

The relative numbers and percentages of explants in the top and bottom layers of the flotation medium were quantified for each of the four experiments (Experiments 1-4), and the data is provided with an average across experiments for each fraction in Table 32. On average in these experiments, 54.7% of the explants were in the top floating fraction, and 42.5% of explants were in the bottom non-floating fraction. To the extent that the explants in the non-floating bottom layer are non-viable and/or non-regenerable, they represent a potential savings to the transformation pipeline if they are discarded and removed. The percentages in Table 32 may not add up to 100% because some explants from the extended bud induction step did not localize into one of the two distinct fractions and/or were not collected, but the number of explants from the total that were not collected and counted in one of the two fractions was less than 3% overall.

TABLE 32
Number and percentage of explants in the floating (top) and
non-floating (bottom) fractions in a 600 mL beaker with
300 mL flotation medium across four independent trials.

| | | | Number | | |
|---|---|---|---|---|---|
| | Number | | of | | |
| | of | Number | Bottom | Percentage | Percentage |
| | Explants | of Top | Non- | Floating | Non-floating |
| Experiment | Tested | Floating | floating | (%) | (%) |
| 1 | 1083 | 573 | 463 | 52.9 | 42.8 |
| 2 | 288 | 148 | 140 | 51.4 | 48.6 |
| 3 | 262 | 158 | 104 | 60.3 | 39.7 |
| 4 | 212 | 130 | 77 | 61.3 | 36.3 |
| Total | 1845 | 1009 | 784 | 54.7 | 42.5 |

### Larger Scale Experiments

Different container sizes can be used as flotation vessels provided that they have sufficient depth for separation of the explants and enough top surface area to accommodate the number of explants to be separated in the vessel. Larger flotation vessels can provide a larger top surface area for the flotation medium in the flotation vessel that allows for separation of a greater number of explants based on flotation.

Three experiments (Experiments 5-7) were conducted in which seed-derived embryo explants were inoculated with Agrobacterium carrying a Ti plasmid contained the same EPSPS expression cassette as described above for the Small Scale Experiments. In addition, the Ti plasmids in Experiments 6 and 7 contained an additional expression cassette encoding either a gene of interest, or a beta-glucuronidase (uidA) driven by a constitutive promoter. In each of these experiments, a Vivi® tissue culture vessel was used as a flotation container and filled with about 2.5 L of flotation medium to a depth of about 3 inches. Approximately 2285 explants were added to the flotation medium for each experiment and incubated for about 10 minutes at ambient temperature until two distinct layers of explant material formed in the flotation vessel including a top layer floating at or near the top of the flotation medium and a bottom layer that sank toward the bottom of the vessel and flotation medium (see, e.g., FIG. 9B).

The relative numbers and percentages of explants in the top and bottom layers in the flotation medium were quantified for each experiment, and the data is provided with an average across experiments for each fraction in Table 33. On average in these experiments, 51.3% of the explants were in the top floating fraction and 48.5% of explants were in the bottom non-floating fraction. These bulk flotation results are consistent with the smaller flotation results described in Small Scale Experiments and indicate that different sizes of flotation vessels may be used to separate seed excised explants after culturing through the bud induction step depending on the size of the experiments, with larger flotation vessels providing a larger top surface area of the flotation medium in the flotation vessel being able to separate a greater number of explants.

TABLE 33
Number and percentage of explants in the floating (top)
and non-floating (bottom) fractions in a Vivi ® tissue
culture vessel across three independent trials.

| | Number | | Number | | |
|---|---|---|---|---|---|
| | of | Number | Bottom | Percentage | Percentage |
| | Explants | of Top | Non- | Floating | Non-floating |
| Experiment | Tested | Floating | floating | (%) | (%) |
| 5 | 2078 | 1143 | 935 | 55.0 | 45.0 |
| 6 | 3607 | 1553 | 2054 | 43.1 | 56.9 |
| 7 | 2097 | 1294 | 803 | 61.7 | 38.3 |
| Total | 7782 | 3990 | 3792 | 51.3 | 48.7 |

### Example 19

### Flotation Enriches Regenerable Explants in Top Floating Layer

The relative plugging frequencies for explants from the top and bottom layers were determined following their separation in the flotation step. After culturing, separation and being blotted dry as described in Example 18, the explants from the top and bottom fractions were transferred to regeneration media to produce regenerated plants for side-by side comparison of their regeneration frequencies and to confirm that the top layer contained more viable greening explants with multiple bud formation, while the bottom layer contained mostly non-viable or dying explants that were brown or darker colored. Phenotypically normal plants following the regeneration step were transferred into soil plugs (plugging). Plants that were not phenotypically normal were not plugged and were removed from further use and testing. Based on the number of phenotypically normal plants that reached the plugging step, the plugging frequency was calculated for each fraction of explants (see Table 34). For a given experiment, the plugging frequency (%) is defined as the number of regenerated plants that were plugged for a given fraction or protocol in an experiment divided by the total number of inoculated explants in the experiment, multiplied by 100. Therefore, the plugging frequency provides an indication of the percentage of explants for each fraction, protocol and experiment that produce phenotypically normal and viable plants following regeneration that may potentially have a desirable transformation event or edit.

Table 34 provides a summary of 40 experiments with (1) 73,630 inoculated explants using the modified protocol with the additional flotation step performed between the extended bud induction and regeneration steps, or (2) 232,959 inoculated explants using the standard protocol without the flotation step, in which many different transformation constructs were used over a period of about two months. As shown in Table 34 across these experiments, explants collected from the top floating layer of the flotation medium had an average plugging frequency of about 1.06%. In contrast, explants collected from the non-floating bottom layer of the flotation medium had a very low plugging frequency of about 0.08%. In control experiments using the standard protocol, cultured explants were transferred to regeneration media following extended bud induction without the flotation step. Explants advanced to regeneration media according to the control protocol (without the flotation step) had a plugging frequency of about 0.86%. Thus, the plugging frequency for explants from the top floating layer in the modified protocol was similar to, or somewhat higher than, the plugging frequency for explants in the control or standard protocol without the flotation step.

TABLE 34
Plugging frequency for regeneration of explants in the top floating
layer and bottom non-floating layer and in the control protocol.

| | | | | Number |
|---|---|---|---|---|
| | | Plugging | Number | Regenerated |
| | | Frequency | Explants | Plants |
| Protocol | Explant Fraction | (%) | Tested | Plugged |
| Flotation | Floating Top Layer | 1.06 | 66,130 | 703 |
| Protocol | Non-floating | 0.08 | 7,500 | 60 |
| | Bottom Layer | | | |
| Control | N/A | 0.86 | 232,959 | 1998 |
| Protocol | | | | |

While the modified protocol with the flotation step may not dramatically improve overall regeneration or plugging frequency relative to the control protocol, a much smaller number of cultured explants were transferred to the subsequent regeneration step from the top floating layer (e.g., about 42-49% less by removing the bottom layer of explant material from the total based on the results in Tables 32 and 33), relative to the control protocol in which most or all cultured explants from the bud induction steps would be transferred.

These results demonstrate that the flotation step selects and enriches for viable and regenerable explants in the top floating layer, which increases the proportion of explants that are moved to plugging from the top layer by removing non-viable and non-regenerable explants at this stage. Thus, the additional flotation step has the potential to provide significant time, space and cost savings in a transformation or editing pipeline without sacrificing plugging or regeneration frequency, and to increase pipeline throughput.

### Example 20

### Single Copy and Contamination Rates Between Standard and Flotation Protocols

To evaluate whether the additional flotation step would increase or spread contamination due to combining explants from multiple culture containers after extended bud induction into the flotation medium in fewer or single flotation vessels, explant cultures were visually inspected for contamination after the flotation step with the modified flotation protocol or after extended bud induction with the standard protocol. A total of 26 experiments with 70,680 explants from the top flotation layer in the modified flotation protocol and 242,478 explants after extended bud induction step in the standard protocol were examined. As shown in Table 35, the percentage of explants from the standard protocol with visible contamination was about 1.56%, whereas the percentage of explants in the top fraction from the flotation protocol with visible contamination was about 2.90%. There was no statistically significant difference in the percentage of explants with contamination between the standard and flotation protocols.

TABLE 35
Percent of explants showing contamination after extended bud
induction in the standard protocol or after separation of
top flotation layer in the modified flotation protocol.

| | | Total | | Percentage |
|---|---|---|---|---|
| | Number of | Number | Number | Contaminated |
| Treatment | Experiments | of Explants | Contaminated | (%) |
| Standard | 26 | 242,478 | 3,776 | 1.56 |
| Flotation | 26 | 70,680 | 2,052 | 2.90 |

To ensure that the additional flotation step would not negatively affect the frequency of single copy transgene insertion events, a quantitative real time TaqMan® PCR assay was used to determine the transgene copy number in transformed plants. Briefly, the TaqMan® assay is a PCR-based genotyping method that uses florescent probes to determine transgene copy number. In this experiment, leaf samples from seedlings regenerated from explants cultured according to either the standard or modified flotation protocol from multiple experiments were collected and subjected to the amplification assay. The copy number of either the CP4 EPSPS and/or the uidA transgene was assayed, and the frequency of events with a single copy number of the transgene was calculated, which is defined as the number of events containing a single copy of the transgene divided by the total number of events tested, multiplied by 100. As shown in Table 36, single copy number frequency was 48.6% for the standard protocol and 43.1% for the modified protocol with the flotation step. There was no statistically significant difference in the single copy number frequencies between the standard and flotation protocols.

TABLE 36
Percentage of single copy transgenic events
in standard and flotation protocols.

| | | Total | Number of | Single Copy |
|---|---|---|---|---|
| | | Number | Single | Number |
| | Number of | of | Copy | Frequency |
| Treatment | Experiments | Events | Events | (%) |
| Standard | 27 | 2,028 | 985 | 48.6% |
| Floatation | 27 | 691 | 298 | 43.1% |

These results indicate that the singly copy number frequency and the contamination frequency were both similar using either the modified flotation protocol or the standard protocol. Thus, the addition of the flotation step did not negatively affect the quality of the explants or transgenic events in these experiments, even though the flotation protocol required advancement of a substantially smaller number of explants to the regeneration and subsequent controlled environment steps to achieve a similar plugging frequency.

### Example 21

### Explant Floatation Density on Contamination Rate and Plugging Frequency

It is desirable to include more cultured explants in a floatation vessel in a transformation production setting to increase the throughput and reduce cost. To determine if increased density of cultured explants in a floatation vessel increases contamination frequency and affects plugging frequency, eight experiments/replications were conducted, each consisting of four treatments: Treatment 1 is a control without floatation, in which about 171 cultured explants were placed into one Vivi vessel for regeneration; Treatments 2-4 contained increased number of cultured explants per floatation vessel (i.e., increased density), thus decreased number of vessels for the same number of cultured explants. In these experiments, seed-derived embryo explants were inoculated with Agrobacterium harboring a Ti plasmid containing an EPSPS expression cassette under the control of a constitutive promoter for selection of transgenic events. The experiments followed the same protocol as described in the preceding Example using Vivi® tissue culture vessel filled with 2.5 L of flotation media to a depth of 2-4 inches. Explants from the top layer of the floatation vessel were collected and transferred to selection and regeneration media. Following regeneration, phenotypically normal plants were plugged and the plugging frequency calculated. Explants were visually inspected for contamination, and the contamination frequency was calculated.

Table 37 is a summary of the contamination rate from the eight experiments. The results show that similar contamination rates were observed across all treatments. Increasing floatation density did not impact contamination rate. From the same experiments, data on plugging frequency was collected and subjected to statistical analysis as seen in Table 38. The results show that the best plugging frequency was obtained with the control, and that there was a slight reduction in plugging frequency for the floatation treatments. Furthermore, similar plugging frequency was observed regardless of the explant floatation density (Table 38).

TABLE 37
Summary of 8 experiments on contamination rates from different floatation densities

| | # Cultured | Floatation Density | # Floatation | Total # | Contamination |
|---|---|---|---|---|---|
| Treatment | Explants/Expt | (#Explant/Vessel) | Vessel/Expt | Explants | Rate (%) |
| 1 (control) | 2280 | No floatation | N/A | 18240 | 2.18 |
| 2 | 2280 | 570 | 4 | 18240 | 1.25 |
| 3 | 2280 | 855 | 3 | 18240 | 3.12 |
| 4 | 2280 | 1140 | 2 | 18240 | 1.25 |

TABLE 38
Summary of transformation plugging frequency from different floatation densities

| | | Floatation | # | | Plugging |
|---|---|---|---|---|---|
| | # Cultured | Density (# | Floatation | Total # | Frequency |
| Treatment | Explants/Expt | Explant/Vessel) | Vessel/Expt | Explants | (%) |
| 1 (control) | 2280 | No floatation | N/A | 18240 | 1.94 |
| 2 | 2280 | 570 | 4 | 18240 | 1.43 |
| 3 | 2280 | 855 | 3 | 18240 | 1.54 |
| 4 | 2280 | 1140 | 2 | 18240 | 1.36 |

To further test the effect of explant floatation density on plugging frequency, additional experiments were conducted. Cultured explants were floated at densities of 285, 570, 855, 1140 and 2280 per floatation vessel. An additional treatment included a floatation density of 2280 with agitation by a stir bar. A standard control was also included, in which the cultured explants did not go through the floatation step. As shown in Table 39, the best plugging frequency was achieved without the floatation step. There was no significant difference in plugging frequencies among the different floatation treatments, which is consistent with the results described in Tables 37 and 38.

TABLE 39
Plugging and contamination frequencies of
explants at different floatation densities

| | Floatation | | Plugging | |
|---|---|---|---|---|
| #Cultured | Density (# | # | Frequency | Contamination |
| Explants/Expt | Explant/Vessel) | Experiment | (%) | Rate (%) |
| 2280 | Control | 4 | 1.44% | 7.50% |
| | (No floatation) | | | |
| 2280 | 285 | 4 | 1.15% | 3.75% |
| 2280 | 570 | 4 | 0.94% | 2.27% |
| 2280 | 855 | 4 | 1.00% | 3.13% |
| 2280 | 1140 | 4 | 1.01% | 0.00% |
| 2280 | 2280 | 4 | 0.82% | 1.88% |
| 2280 | 2280 with | 4 | 1.03% | 1.25% |
| | agitation | | | |

### Example 22

### Duration of Explant Floatation on Plugging Frequency

To test whether the duration of the explant floatation affected plugging and contamination frequencies, five independent experiments were conducted. Each experiment included five different treatments/floatation durations: 30 minutes, 1 hour, 2 hours, 4 hours, and overnight, and followed the same experimental protocol as described in the preceding examples except for the changes in floatation duration. Explants were collected from the top floating layer following floatation and placed on selection and regeneration media. Regenerated phenotypically normal plants were transferred to plugging media, and the plugging frequency was calculated. Contamination frequency was determined by visually inspecting explants for contamination prior to plugging.

Similar plugging frequencies were observed with shorter floatation durations (0.5-2 hours). As the duration further increased, there seemed to be a decrease in plugging frequency, with overnight floatation being the lowest (Table 40). The contamination frequencies for all floatation durations were within acceptable ranges.

TABLE 40
Floatation duration on plugging and contamination frequencies

| Float | | | Plugging | |
|---|---|---|---|---|
| Duration | # | # | Frequency | Contamination |
| (hr) | Explants/Experiment | Experiments | (%) | Rate (%) |
| 0.5 | 2280 | 5 | 0.94 | 3.00 |
| 1 | 2280 | 5 | 0.97 | 1.50 |
| 2 | 2280 | 5 | 0.91 | 3.03 |
| 4 | 2280 | 5 | 0.82 | 0.00 |
| Overnight | 2280 | 5 | 0.62 | 1.50 |

### Example 23

### Floatation Medium Compositions on Plugging Frequency

To determine if the floatation medium's composition affects plugging frequency, two sets of experiments were conducted. In the first set of experiments, water was used as the floatation medium, and contained different fungicides and/or antibiotics: Absolute® 500 SC fungicide (Bayer Crop Science, St. Louis, MO 63167, USA); Cleary's® (Cleary Chemical Corporation, Dayton, NJ 08810, UAS); Cleary's® with carbenicillin, cefotaxime and timentin; and Absolute® with carbenicillin, cefotaxime and timentin. The experiments followed the same experimental protocol as described in the preceding examples except for the floatation medium. Explants were collected from the top floating layer following floatation and placed on selection and regeneration media. Regenerated phenotypically normal plants were transferred to plugging media, and the plugging frequency was calculated. Contamination frequency was determined by visually inspecting explants for contamination prior to plugging. The results indicate that plugging frequency was not significantly different among the four different treatments (Table 41).

TABLE 41
Fungicides and antibiotics in floatation
water on plugging frequency

| Floatation | | | Plugging | |
|---|---|---|---|---|
| Medium | #Explants/ | # | Frequency | Contamination |
| Additives | Expt | Experiments | (%) | Rate (%) |
| Absolute ® 500 | 2280 | 4 | 0.85 | 5.63 |
| Cleary's ® | 2280 | 4 | 0.97 | 3.75 |
| Cleary's ® + | 2280 | 4 | 0.90 | 3.13 |
| Antibiotics | | | | |
| Absolute ® | 2280 | 4 | 1.10 | 2.5 |
| 500 + | | | | |
| Antibiotics | | | | |

In the second set of experiments, three types of floatation media were tested: water, 4289, and 5003. In addition, floatation in 5003 media followed by a blotting step to remove excess medium from the explants was also tested. Explants were collected from the top floating layer following floatation and placed on selection and regeneration media. Regenerated phenotypically normal plants were transferred to plugging media, and the plugging frequency was calculated. Contamination frequency was determined by visually inspecting explants for contamination prior to plugging.

As shown in Table 42, floatation in water had the lowest plugging frequency of 0.63%. There was no significant difference in plugging frequencies among the rest of the treatments, i.e., the different media and the extra step of blot dry did not seem to affect plugging frequency. The contamination frequency for all experimental categories was within the acceptable parameters.

TABLE 42
Different floatation media on plugging frequency

| | | | Plugging | |
|---|---|---|---|---|
| Floatation | # | # | Frequency | Contamination |
| Medium | Explants/Expt | Experiments | (%) | Rate (%) |
| Water + Absolute ® | 2280 | 4 | 0.63 | 1.25 |
| 500 | | | | |
| 4289¹ + | 2280 | 4 | 0.94 | 0 |
| Absolute ® 500 | | | | |
| 5003² + | 2280 | 4 | 0.86 | 1.25 |
| Absolute ® 500 | | | | |
| 5003² + | 2280 | 4 | 0.77 | 3.75 |
| Absolute ® | | | | |
| 500 with Blot Dry | | | | |

¹Medium 4289: MS basal salts with B5 vitamins, 60 g/L sucrose, 0.5 g/L glutamine, 1 g/L NZ Amine-A, 0.69 g/L proline, 2 mg/L glycine, 1.95 mg/L MES, 1 mg/L Absolute ® 500, pH 5.8.
²Medium 5003: LM Woody Plant Medium salts and vitamins, 0.03 g/L Clearys 3336 WP, 30 g/L sucrose, 0.69 g/L proline, 2 mg/L glycine, 1 g/L MES, 1 mg/L Absolute ® 500, pH 5.8.

What is claimed is:

1. A method of enriching regenerable monocot seed embryo explants comprising:
culturing a plurality of monocot seed embryo explants comprising meristematic tissue in contact with a bud induction medium comprising a first auxin and a first cytokinin,
contacting the plurality of monocot seed embryo explants with a flotation medium contained within a flotation vessel after culturing the monocot seed embryo explants in contact with the bud induction medium to separate the plurality of monocot seed embryo explants into a first portion of the plurality of monocot seed embryo explants present within a top region of the flotation medium and a second portion of the plurality of monocot seed embryo explants present within a bottom region of the flotation medium,
selectively collecting all or part of the first portion of the plurality of monocot seed embryo explants from a top region of the flotation medium.

2. The method of claim 1, wherein the monocot seed embryo explants are physically agitated while in contact with the flotation medium.

3. The method of claim 1 or 2, wherein the monocot seed embryo explants are sonicated while in contact with the flotation medium.

4. The method of any one of claims 1-3, wherein the flotation medium comprises water.

5. The method of any one of claims 1-4, wherein the flotation medium is an aqueous solution comprising at least one salt.

6. The method of any one of claims 1-5, wherein the flotation medium comprises an antimicrobial agent.

7. The method of any one of claims 1-6, wherein the depth of the flotation medium in the flotation vessel is in a range from about 0.5 inches to about 24 inches, from about 1 inch to about 10 inches, from about 1 inch to about 5 inches, from about 1 inch to about 4 inches, from about 1.5 inches to about 5 inches, from about 2 inches to about 5 inches, from about 2 inches to about 4 inches, or from about 2 inches to about 3 inches.

8. The method of any one of claims 1-7, wherein the plurality of monocot seed embryo explants are contacted with the flotation medium in the flotation vessel at a density relative to the surface area of the upper surface of the flotation medium in the flotation vessel in a range from about 1 to about 100 monocot seed embryo explants per square inch, from about 5 to about 50 monocot seed embryo explants per square inch, from about 10 to about 50 monocot seed embryo explants per square inch, from about 15 to about 40 monocot seed embryo explants per square inch, from about 15 to about 30 monocot seed embryo explants per square inch, from about 20 to about 30 monocot seed embryo explants per square inch, or from about 25 to about 30 monocot seed embryo explants per square inch.

9. The method of any one of claims 1-8, wherein the top region of the flotation medium is separated from the bottom region of the flotation medium by an average gap distance.

10. The method of claim 9, wherein the average gap distance is in a range from about 0.25 inches to about 12 inches, from about 0.5 inches to about 10 inches, from about 0.5 inches to about 5 inches, from about 1 inch to about 5 inches, from about 1.5 inches to about 5 inches, from about 2 inches to about 5 inches, from about 1 inch to about 4 inches, from about 1 inch to about 3 inches, or from about 1 inch to about 2 inches.

11. The method of any one of claims 1-10, wherein the plurality of monocot seed embryo explants are contacted with the flotation medium for a period of time in a range from about 5 seconds to about 12 hours, from about 10 seconds to about 6 hours, from about 10 seconds to about 3 hours, from about 10 seconds to about 1 hour, from about 10 seconds to about 30 minutes, from about 10 seconds to about 10 minutes, from about 10 seconds to about 5 minutes, from about 15 seconds to about 2 minutes, or from about 15 seconds to about 1 minute.

12. The method of any one of claims 1-11, further comprising:
transferring the collected first portion of the plurality of monocot seed embryo explants to a regeneration medium.

13. The method of claim 12, wherein the collected first portion of the plurality of monocot seed embryo explants are transferred directly from the flotation medium to the regeneration medium.

14. The method of claim 12, wherein the collected first portion of the plurality of monocot seed embryo explants are transferred indirectly from the flotation medium to the regeneration medium.

15. The method of claim 14, wherein the collected first portion of the plurality of monocot seed embryo explants are blotted prior to being transferred to the regeneration medium.

16. The method of any one of claims 12-15, further comprising:
regenerating at least one monocot plant or plant part from the collected first portion of the plurality of monocot seed embryo explants in contact with the regeneration medium.

17. The method of claim 16, wherein the at least one monocot plant or plant part comprises at least one genetically modified plant or plant part.

18. The method of claim 16 or 17, wherein the plant part is a shoot or a root.

19. The method of any one of claims 16-18, wherein the at least one monocot plant or plant part is regenerated in contact with the regeneration medium at a temperature in a range from about 20° C. to about 32° C., from about 25° C. to about 29° C., or from about 27° C. to about 28° C.

20. The method of any one of claims 16-19, wherein the at least one monocot plant or plant part is regenerated in contact with a regeneration medium for a time period in a range from about 20 days to about 50 days or from about 28 days to about 42 days.

21. The method of any one of claims 16-20, wherein the regeneration medium has a low salt concentration.

22. The method of any one of claims 16-21, wherein the regeneration medium does not contain an auxin or a cytokinin.

23. The method of any one of claims 16-22, wherein the plurality of monocot seed embryo explants and the at least one genetically modified monocot plant or plant part are cultured and regenerated without producing a callus tissue culture.

24. The method of any one of claims 16-23, wherein the regeneration medium comprises a selection agent.

25. The method of claim 24, wherein the selection agent is selected from the group consisting of kanamycin, paromomycin, hygromycin B, spectinomycin, streptomycin, gentamycin, glyphosate, glufosinate, phosphinothricin, bromoxynil, bialaphos, dicamba, imidazolinone, and sulfonylurea.

26. The method of any one of claims 16-25, wherein the at least one monocot plant or plant part is non-chimeric.

27. The method of any one of claims 16-26, wherein the monocot plant or plant part is a corn plant or plant part, a wheat plant or plant part, a rice plant or plant part, a barley plant or plant part, a turfgrass plant or plant part, or a sorghum plant or plant part.

28. The method of claim 27, wherein the monocot plant or plant part is a corn plant or plant part.

29. The method of claim 27, wherein the monocot plant or plant part is a wheat plant or plant part.

30. The method of any one of claims 16-29, further comprising:
preparing a plant part from a regenerated plant.

31. The method of any one of claims 16-30, further comprising:
selectively collecting all or part of the second portion of the plurality of monocot seed embryo explants from the bottom region of the flotation medium,
transferring the collected second portion of the plurality of monocot seed embryo explants to a regeneration medium, and
regenerating at least one genetically modified monocot plant or plant part from the collected second portion of the plurality of monocot seed embryo explants in contact with the regeneration medium,
wherein the first portion of the plurality of monocot seed embryo explants has a higher plugging or regeneration frequency than the second portion of the plurality of monocot seed embryo explants.

32. The method of claim 31, wherein the plugging or regeneration frequency is measured as the number of plugged or regenerated plants from the collected first portion or the second portion of the plurality of monocot seed embryo explants divided by the total number of monocot seed embryo explants in the plurality of monocot seed embryo explants.

33. The method of claim 31, wherein the plugging or regeneration frequency is measured as the number of plugged or regenerated plants from the first portion or the second portion of the plurality of monocot seed embryo explants divided by the number of monocot seed embryo explants in the first portion or the second portion, respectively, that are transferred to the regeneration medium.

34. The method of any one of claims 31-33, wherein the plugging or regeneration frequency of the first portion of the plurality of monocot seed embryo explants is at least two-fold, at least three-fold, at least four-fold, at least five-fold, at least ten-fold, at least fifteen-fold, at least twenty-fold, at least twenty five-fold, at least thirty-fold, at least forty-fold, at least fifty-fold, at least seventy five-fold, or at least one hundred-fold higher or greater than the plugging or regeneration frequency of the second portion of the plurality of monocot seed embryo explants.

35. The method of any one of claims 16-34, wherein the first portion of the plurality of monocot seed embryo explants has a same or similar or moderately reduced plugging or regeneration frequency as a control population of monocot seed embryo explants, wherein the control population of monocot seed embryo explants is cultured using a control protocol in which the control population of monocot seed embryo explants is not contacted with a flotation medium to separate the control population of monocot seed embryo explants into top and bottom portions prior to regeneration of one or more plants from the control population.

36. The method of claim 35, wherein the number of monocot seed embryo explants in the first portion or collected first portion is less than the number of monocot seed embryo explants in the control population of monocot seed embryo explants.

37. The method of claim 36, wherein the number of monocot seed embryo explants in the first portion or collected first portion is at least 5% less, at least 10% less, at least 15% less, at least 20% less, at least 25% less, at least 30% less, at least 35% less, at least 40% less, at least 45% less, or at least 50% less than the number of monocot seed embryo explants in the control population of monocot seed embryo explants.

38. The method of any one of claims 1-37, further comprising:
introducing a heterologous polynucleotide molecule into at least one cell of a monocot seed embryo explant of the plurality of monocot seed embryo explants.

39. The method of claim 38, wherein the heterologous polynucleotide molecule is introduced into the monocot seed embryo explant via bacteria-mediated transformation or microprojectile bombardment.

40. The method of claim 38 or 39, wherein the heterologous polynucleotide molecule is introduced into the monocot seed embryo explant via Rhizobiales or Agrobacterium mediated transformation.

41. The method of claim 40, wherein the heterologous polynucleotide molecule is introduced into the monocot seed embryo explant by inoculating the plurality of monocot seed embryo explants in contact with an inoculation medium comprising a Rhizobiales bacterium competent to transform the at least one cell of the monocot seed embryo explant with the heterologous polynucleotide molecule.

42. The method of claim 40 or 41, wherein the Rhizobiales bacterium is selected from the group consisting of:
a) a Rhizobiaceae, a Phyllobacteriaceae, a Brucellaceae, a Bradyrhizobiaceae, and a Xanthobacteraceae bacterium; or
b) an Agrobacterium, a Rhizobium, a Sinorhizobium, a Mesorhizobium, a Phyllobacterium, an Ochrobactrum, a Bradyrhizobium, and an Azorhizobium bacterium.

43. The method of any one of claims 40-42, further comprising:
co-culturing the plurality of monocot seed embryo explants with the Rhizobiales or Agrobacterium in contact with a co-culture medium.

44. The method of claim 43, wherein the plurality of monocot seed embryo explants is co-cultured at a temperature of about 15° C. to about 25° C. or about 20° C.

45. The method of claim 43 or 44, wherein the plurality of monocot seed embryo explants is co-cultured for a time period in a range from about 2 days to about 10 days or from about 5 days to about 7 days.

46. The method of any one of claims 43-45, wherein the co-culture medium does not contain an auxin or a cytokinin.

47. The method of any one of claims 43-46, wherein the co-culture medium does not contain a surfactant.

48. The method of any one of claims 43-47, wherein the co-culture medium is in contact with a paper substrate wetted with the co-culture medium.

49. The method of any one of claims 1-48, wherein the plurality of monocot seed embryo explants is cultured in contact with the bud induction medium for a time period in a range from about 2 days to about 14 days or from about 6 days to about 8 days.

50. The method of any one of claims 1-49, wherein the plurality of monocot seed embryo explants is cultured in contact with the bud induction medium at a temperature in a range selected from the group consisting of about 20° C. to about 40° C., about 25° C. to about 30° C., about 30° C. to about 40° C., about 30° C. to about 37° C., and about 33° C. to about 35° C.

51. The method of any one of claims 1-50, wherein the bud induction medium comprises a high cytokinin to auxin ratio.

52. The method of any one of claims 1-51, wherein the first auxin in the bud induction medium is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba).

53. The method of claim 52, wherein the first auxin in the bud induction medium is 2,4-dichlorophenoxy-acetic acid (2,4-D).

54. The method of claim 52, wherein the first auxin in the bud induction medium is 4-amino-3,5,6-trichloro-picolinic acid (picloram).

55. The method of any one of claims 1-54, wherein the concentration of the first auxin in the bud induction medium is from about 0.02 mg/L to about 25 mg/L or is from about 1 mg/L to about 2 mg/L.

56. The method of any one of claims 1-55, wherein the first cytokinin in the bud induction medium is selected from the group consisting of: 6-benzylaminopurine (BAP), thidiazuron (TDZ), kinetin, zeatin, diphenyl urea (DPU), 6-(gamma,gamma-dimethylallylamino) purine (2iP), and meta-topolin.

57. The method of claim 56, wherein the first cytokinin in the bud induction medium is 6-benzylaminopurine (BAP).

58. The method of claim 56, wherein the first cytokinin in the bud induction medium is thidiazuron (TDZ).

59. The method of any one of claims 1-58, wherein the concentration of the first cytokinin in the bud induction medium is in a range from about 0.1 mg/L to about 50 mg/L.

60. The method of claim 59, wherein the first cytokinin is BAP and the concentration of the BAP in the bud induction medium is about 10 mg/L.

61. The method of claim 59, wherein the first cytokinin is TDZ and the concentration of the TDZ in the bud induction medium is about 2 mg/L.

62. The method of any one of claims 1-61, wherein the bud induction medium is a solid medium.

63. The method of any one of claims 1-62, further comprising:
culturing the plurality of monocot seed embryo explants in contact with a second bud induction medium comprising (i) the first auxin or a second auxin and (ii) the first cytokinin or a second cytokinin, wherein the plurality of monocot seed embryo explants are contacted with the flotation medium after culturing the monocot seed embryo explants in contact with the second bud induction medium.

64. The method of claim 63, wherein the plurality of monocot seed embryo explants is cultured in contact with the second bud induction medium for a time period in a range from about 4 days to about 28 days or from about 7 to about 14 days.

65. The method of claim 63 or 64, wherein the plurality of monocot seed embryo explants is cultured in contact with the second bud induction medium at a temperature in a range from about 20° C. to about 32° C., from about 25° C. to about 29° C., or from about 27° C. to about 28° C.

66. The method of any one of claims 63-65, wherein the second bud induction medium comprises a high cytokinin to auxin ratio.

67. The method of any one of claims 63-66, wherein the second bud induction medium comprises:
a) the first auxin and the first cytokinin;
b) the first auxin and the second cytokinin;
c) the second auxin and the first cytokinin; or
d) the second auxin and the second cytokinin.

68. The method of any one of claims 63-67, wherein the first auxin or the second auxin in the second bud induction medium is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba).

69. The method of claim 68, wherein the first auxin or the second auxin in the second bud induction medium is 2,4-dichlorophenoxy-acetic acid (2,4-D).

70. The method of claim 68, wherein the first auxin or the second auxin in the second bud induction medium is 4-amino-3,5,6-trichloro-picolinic acid (picloram).

71. The method of any one of claims 63-70, wherein the first cytokinin or the second cytokinin in the second bud induction medium is selected from the group consisting of: 6-benzylaminopurine (BAP), thidiazuron (TDZ), kinetin, zeatin, diphenyl urea (DPU), 6-(gamma,gamma-dimethylallylamino) purine (2iP), and meta-topolin.

72. The method of claim 71, wherein the first cytokinin or the second cytokinin in the second bud induction medium is 6-benzylaminopurine (BAP).

73. The method of claim 71, wherein the first cytokinin or the second cytokinin in the second bud induction medium is thidiazuron (TDZ).

74. The method of any one of claims 63-73, wherein the second bud induction medium comprises 4-amino-3,5,6-trichloro-picolinic acid (picloram) and thidiazuron (TDZ), or 2,4-dichlorophenoxy-acetic acid (2,4-D) and thidiazuron (TDZ).

75. The method of any one of claims 63-74, wherein the concentration of the first cytokinin or the second cytokinin in the second bud induction medium is in a range from about 0.1 mg/L to about 50 mg/L, from about 0.1 mg/L to about 25 mg/L, or from about 2 mg/L to about 10 mg/L.

76. The method of claim 75, wherein the first cytokinin or the second cytokinin is TDZ and the concentration of TDZ in the second bud induction medium is about 2 mg/L; or the first cytokinin or the second cytokinin is BAP and the concentration of BAP in the second bud induction medium is about 10 mg/L.

77. The method of any one of claims 63-76, wherein the concentration of the first auxin or the second auxin in the second bud induction medium is about 0.01 mg/L to about 25 mg/L, about 0.02 mg/L to about 10 mg/L, or about 1 mg/L to about 2 mg/L.

78. The method of any one of claims 63-77, wherein the second bud induction medium is a solid medium.

79. The method of any one of claims 63-78, wherein the heterologous polynucleotide molecule comprises a selectable marker gene, wherein the second bud induction medium comprises a selection agent, and wherein the selectable marker gene provides resistance in a plant to the selection agent.

80. The method of claim 79, wherein the selection agent is selected from the group consisting of kanamycin, paromomycin, hygromycin B, spectinomycin, streptomycin, gentamycin, glyphosate, glufosinate, phosphinothricin, bromoxynil, bialaphos, dicamba, imidazolinone, and sulfonylurea.

81. The method of any one of claims 63-80, wherein the bud induction medium comprises the first auxin and the second bud induction medium comprises the second auxin, and wherein the first auxin is different than the second auxin.

82. The method of any one of claims 63-81, wherein the bud induction medium comprises the first cytokinin and the second bud induction medium comprises the second cytokinin, and wherein the first cytokinin is different than the second cytokinin.

83. The method of any one of claims 63-82, wherein the first cytokinin in the bud induction medium is 6-benzylaminopurine (BAP) and the second cytokinin in the second bud induction medium is thidiazuron (TDZ).

84. The method of any one of claims 63-83, wherein the first auxin in the bud induction medium is 2,4-dichlorophenoxy-acetic acid (2,4-D) and the second auxin in the second bud induction medium is 4-amino-3,5,6-trichloro-picolinic acid (picloram).

85. The method of any one of claims 63-84, wherein the second bud induction medium comprises the second auxin and the second cytokinin, wherein the second auxin is 4-amino-3,5,6-trichloro-picolinic acid (picloram), wherein the concentration of 4-amino-3,5,6-trichloro-picolinic acid (picloram) is in a range from about 0.1 mg/L to about 10.0 mg/L or from about 0.5 mg/L to about 4 mg/L, and wherein the second cytokinin is thidiazuron (TDZ), wherein the concentration of thidiazuron (TDZ) in the second bud induction medium is in a range from about 0.5 mg/L to about 15 mg/L or from about 1 mg/L to about 4 mg/L.

86. The method of any one of claims 63-85, wherein the first auxin is 2,4-dichlorophenoxy-acetic acid (2,4-D), wherein the concentration of 2,4-dichlorophenoxy-acetic acid (2,4-D) in the bud induction medium is in a range from about 0.1 mg/L to about 10 mg/L or from about 0.1 mg/L to about 4 mg/L, wherein the first cytokinin is 6-benzylaminopurine (BAP), and wherein the concentration of 6-benzylaminopurine (BAP) in the bud induction medium is in a range from about 1 mg/L to about 25 mg/L or about 2 mg/L to about 20 mg/L.

87. The method of any one of claims 1-86, wherein the plurality of monocot seed embryo explants comprises a plurality of corn seed embryo explants, a wheat seed embryo explants, a rice seed embryo explants, a barley seed embryo explants, a turfgrass seed embryo explants, or sorghum seed embryo explants.

88. The method of claim 87, the plurality of monocot seed embryo explants comprises a plurality of corn seed embryo explants.

89. The method of claim 87, the plurality of monocot seed embryo explants comprises a plurality of wheat seed embryo explants.

90. The method of any one of claims 1-89, wherein the plurality of monocot seed embryo explants is prepared from monocot seeds under conditions wherein the plurality of monocot seed embryo explants do not germinate and remain viable and competent for genetic transformation.

91. The method of any one of claims 1-90, wherein the monocot seeds from which the plurality of monocot seed embryo explants is prepared have an internal moisture content in a range from about 3% to about 25%.

92. The method of any one of claims 1-91, wherein the plurality of monocot seed embryo explants is a plurality of dry mature corn seed embryo explants, or wherein each of the monocot seed embryo explants has an internal moisture content in a range from about 3% to about 25% prior to introducing the heterologous polynucleotide molecule.

93. The method of any one of claims 1-92, wherein each of the monocot seed embryo explants comprises the apical portion of the embryo axis lacking the radical, and wherein the remaining portions of the monocot seeds from which the plurality of monocot seed embryo explants is prepared have been substantially removed from the monocot seed embryo explants.

94. The method of any one of claims 1-93, wherein the heterologous polynucleotide molecule comprises a gene of interest or one or more expression cassettes encoding a guide RNA or a site-directed nuclease.

95. The method of any one of claims 1-94, wherein the number of monocot seed embryo explants in the first portion or collected first portion is less than the number of monocot seed embryo explants in the plurality of monocot seed embryo explants.

96. The method of claim 95, wherein the number of monocot seed embryo explants in the first portion or collected first portion is at least 5% less, at least 10% less, at least 15% less, at least 20% less, at least 25% less, at least 30% less, at least 35% less, at least 40% less, at least 45% less, or at least 50% less than the number of monocot seed embryo explants in the plurality of monocot seed embryo explants.

97. A method of enriching regenerable monocot seed embryo explants comprising:
culturing a plurality of monocot seed embryo explants comprising meristematic tissue in contact with a bud induction medium comprising a first auxin and a first cytokinin,
contacting a subset of the monocot seed embryo explants of the plurality of monocot seed embryo explants with a flotation medium contained within a flotation vessel after culturing the monocot seed embryo explants in contact with the bud induction medium to separate the subset of monocot seed embryo explants into a first portion of the plurality of monocot seed embryo explants present within a top region of the flotation medium and a second portion of the plurality of monocot seed embryo explants present within a bottom region of the flotation medium,
selectively collecting the first portion of the subset of monocot seed embryo explants from the top region of the flotation medium, and
regenerating at least one monocot plant or plant part from the collected first portion of the subset of monocot seed embryo explants in contact with the regeneration medium.

98. The method of claim 97, further comprising:
dividing the plurality of cultured monocot seed embryo explants into the subset of monocot seed embryo explants and a second subset of monocot seed embryo explants, and
regenerating at least one monocot plant or plant part from the second subset of monocot seed embryo explants in contact with a regeneration medium using a control protocol in which the second subset of monocot seed embryo explants is not contacted with a flotation medium to separate the second subset of monocot seed embryo explants into a top portion and a bottom portion prior to regenerating the at least one monocot plant or plant part from the second subset of monocot seed embryo explants.

99. The method of claim 98, wherein the first portion of the plurality of monocot seed embryo explants has a same or similar or moderately reduced plugging or regeneration frequency as the second subset of the plurality of monocot seed embryo explants.

100. The method of claim 98 or 99, wherein the number of monocot seed embryo explants in the subset is approximately the same as the number of monocot seed embryo explants in the second subset, and wherein the number of monocot seed embryo explants in the first portion of monocot seed embryo explants is less than the number of monocot seed embryo explants in the second subset of monocot seed embryo explants.

101. The method of claim 100, wherein the number of monocot seed embryo explants in the first portion of monocot seed embryo explants is at least 5% less, at least 10% less, at least 15% less, at least 20% less, at least 25% less, at least 30% less, at least 35% less, at least 40% less, at least 45% less, or at least 50% less than the number of monocot seed embryo explants in the second subset of monocot seed embryo explants.
